(19) United States
(12) Patent Application Publication
(10) Pub. No.: US 2026/0315650 A1
(43) Pub. Date: Oct. 15, 2026

(54) OSTEOINSTRUCTIVE CALCIUM PHOSPHATE CERAMIC MATERIAL

(71) Applicant: Locate Bio Limited, Nottingham (GB)

(72) Inventors: John VON BENECKE, Reading (GB); Kevin SHAKESHEFF; Charles MATTHEWS; Helen COX; Elisa TARSITANO

(21) Appl. No.: 19/476,491

(22) PCT Filed: Apr. 26, 2024
(86) PCT No.: PCT/GB2024/051116

§ 371 Date: Oct. 17, 2025

(30) Foreign Application Priority Data
Apr. 28, 2023 (GB) 2306382.9

(51) Int. Cl. A61L 27/12; A61L 27/56

(52) U.S. Cl. CPC A61L 27/12; A61L 27/56; A61L 2400/08; A61L 2430/02

(57) ABSTRACT

The present invention relates to a method of producing pellets and granules, which are osteoconductive, and may be osteoinstructive or osteoinductive, and uses thereof.

### FIELD OF THE INVENTION

The present invention relates to a method of producing pellets and granules, which are osteoconductive, and may be osteoinstructive or osteoinductive, and uses thereof.

### BACKGROUND

Within the field of regenerative medicine there are many opportunities for new clinical procedures that stimulate and support tissue repair. Examples of clinical opportunities include regeneration of cardiac muscle after an infarction, induction of bone growth in spinal fusion, healing of diabetic foot ulcers and limitation or, perhaps, reversal of damage due to stroke. Examples of tissues where treatment could facilitate healing are brain tissue, liver tissue and pancreatic tissue, amongst others.

One area where tissue healing is important is bone healing, for example for people with bone disorders. Bone healing is a physiological process in which the body facilitates the repair of the bone after an external injury, infection, surgical intervention, or a disease. The physiological healing process can require very long periods and in many cases, it cannot re-establish the original bone properties. For this reason, therapies that accelerate and improve bone healing are of vital importance. Usually, these therapies present osteoconductive, osteoinductive, and osteogenic approaches. In the majority of osteoconductive approaches, a variety of substitutes like gold, stainless steel, titanium, natural/synthetic polymers and ceramics have been tried. The main concerns with the use of these materials for bone reconstruction were their poor ability to vascularise, integrate, and undergo remodelling. This may result in structural failure of the implant under load or pathological changes in the surrounding bone, as seen in stress shielding. The other issues are inflammatory scarring, neoproliferative reaction in the adjacent tissues and infection. As a result of their high osteoinductive potential and remodelling characteristics, bioactive substitutes have been used with promising results. This led to the evolution of tissue engineering techniques (biologically enhanced allografts, cell-based therapies, and gene-based therapies) to treat bone disorders. Tissue engineering has been defined as the application of scientific principles to the design, construction, modification, and growth of living tissue using biomaterials, cells, and factors alone and in combination. It involves the use of osteoconductive biomaterial scaffolds, with osteogenic cell populations and osteoinductive bioactive factors. All these approaches have the potential to significantly increase our ability to treat diseases for which no effective treatment currently exists.

Materials such as Bioactive Glass 45S5, biphasic calcium phosphate (BCP) and β-Tricalcium Phosphate (β-TCP) which have been widely used in bone tissue engineering. Calcium phosphate ceramics have shown great promise to become the biomaterials of the future, because they are safe, found naturally in our body, and integrate with tissues easily. It is known that nanoscale features on implants, such as surface roughness has consistently shown to enhance osteoblast gene expression, and this is leading to an entire field of enhanced metal implants. Similarly, grain and micropore size in Calcium phosphate ceramic biomaterials can greatly enhance osteoconductive properties. Yuan (WO 2007094672A1, 2007) has shown that grain size<1.5 μm are important for ectopic bone growth, and Fellah (2010) has shown that macrophages and giant cells varied by grain size (see for example, Fellah et al., J Biomed Mater Res A 93, 1588-1595, 2010).

Grain and micropore size in calcium phosphate ceramic biomaterials is understood to be primarily controlled by sintering temperature (Bignon, A., et al., Journal of Materials Science: Materials in Medicine, 14 (12), 1089-1097, 2003). Porous calcium phosphate ceramics with nanoscale grains can be fabricated through a single or two-step low-temperature sintering process. The grain size evolution of calcium phosphate ceramics is a function of sintering temperature and time. Getting nanocrystalline ceramics requires lower activation sintering energy, faster sintering rate, and lower initial sintering temperature as compared to microcrystalline ceramics (Zhou et al., Ceramics International 41, 4696-4705, 2015). However, it is still a challenge to control the production of such material to simultaneously provide properties such as macroporosity and microporosity, or further control of surface topography at a submicron scale.

Therefore, despite the advances in the processing of calcium phosphate ceramic biomaterials, there is a need to further improve the provision of such materials that are effective as an osteoconductive material, and potentially osteoinstructive or osteoinductive material.

### SUMMARY OF INVENTION

An aim of the present invention is to provide an improved osteoconductive calcium phosphate ceramic biomaterial, for example for bone repair, which may be osteoinstructive or osteoinductive.

According to a first aspect of the invention there is provided a method of producing osteoconductive pellets, the method comprising:
(i) providing powdered calcium phosphate material mixed with polymer particles to form a feedstock;
(ii) shaping the feedstock to form pellets,
(iii) sintering the pellets, wherein the sintering comprises:
(a) a first stage heating at a temperature and time that is sufficient to burn off substantially all of the polymer and leave calcium phosphate material pellets; and
(b) a second stage heating at a higher temperature of between 950° C. and 1150° C. to sinter the calcium phosphate material pellets, thereby forming osteoconductive pellets with surface topography in the form of 0.2 to 20 micron grains; and optionally
(c) applying a hydrothermal treatment to the osteoconductive pellets to enhance their surface topography.

Advantageously, the method of the present invention is capable of providing key improvements over existing methods. For example, the method provides control of macro-porosity via the pelleting process, which is known to be very important in bone formation. Ripamonti (J. Cell. Mol. Med. Vol 8, No 2, 2004, pp. 169-180) reported bone forming ectopically in non-human primates only in the concave surfaces of macropores. Fukuda et al. (Acta Biomater. 7 (5), 2327-2336, 2011) reported that it was possible to product ectopic bone from titanium implants which has a tube-like structure. Unlike the present invention, previously used techniques, such as sacrificial porogen techniques, are not able to produce tube like macro-porous structures, and further provide simultaneous control of micro-porosity. Whilst is it understood by those skilled in the art how to increase micro-porosity through the control of sintering temperature (the lower the temp, the more micro-porosity), the ability to simultaneously control both macro-porosity and micro-porosity during manufacturing is a particular advantage of the present invention.

Further simultaneous control of surface topography at submicron scale is also available according to the present invention. Whilst is it understood by those skilled in the art how to create submicron features as surface topography, the ability to control this together with macro-porosity and micro-porosity is another advantage. The resulting osteoconductive material has controlled macroporosity, high levels of microporosity and submicron surface topography. This has potential for high protein adsorption, and a favourable in vitro cellular response, including a highly advantageous transition from M0 macrophages to M2. Advantageously, a 6- and 12-week study in an intramuscular defect model in adult sheep was conducted to evaluate the in-vivo response of the pellets, where an osteoinductive effect was demonstrated. The resulting osteoconductive pellets of the first aspect of the invention may otherwise be termed herein as “Certoss™ Conduit”.

According to a second aspect of the present invention, there is provided a method of producing osteoconductive β-TCP granules; the method comprising:
(i) providing β-TCP granules;
(ii) adding α-TCP particles and water to the β-TCP granules to form seeded β-TCP granules; and
(iii) applying a hydrothermal treatment to the seeded β-TCP granules, wherein the hydrothermal treatment comprises heating to a temperature of between about 90° C. and 120° C. in the presence of water to enhance the surface topography of the β-TCP granules, thereby forming the osteoconductive β-TCP granules.

Advantageously, the method of producing osteoconductive β-TCP granules results in β-TCP granules with enhanced surface topography, such as needle-like projections on their surface. This has been shown to improve osteoconductivity, but also shown to produce an osteoinstructive or osteoinductive effect. The resulting osteoconductive granules of the second aspect of the invention may otherwise be termed herein as “Certoss™ Granules”.

According to a further aspect of the invention, there is provided osteoconductive pellets and/or osteoconductive granules formed by the methods of the invention herein. There is provided osteoconductive pellets formed by the method according to the first aspect of the invention. There is provided osteoconductive granules formed by the method according to the second aspect of the invention.

According to a further aspect of the invention, there is provided an osteoconductive pellet, wherein the osteoconductive pellet is microporous and comprises or consists of at least 60% w/w calcium phosphate material, and optionally wherein the osteoconductive pellet comprises one or more concave surfaces, such as a hollow extending therethrough.

According to a further aspect of the invention, there is provided an intermediate pellet suitable for the production of the osteoconductive pellet of the invention, wherein the intermediate pellet comprises or consists of less than 28% w/w polymer particles encapsulated within at least 60% w/w powdered calcium phosphate material.

According to a further aspect of the invention, there is provided an osteoconductive granule, wherein the osteoconductive granule comprises or consists of β-TCP, and further comprises a surface topography comprising needle structures projecting from the surface of the granule.

According to a further aspect of the invention, there is provided an osteoconductive material, wherein the osteoconductive material comprises or consists of a plurality of osteoconductive pellets and/or granules according to the invention.

According to a further aspect of the invention, there is provided a composition comprising the osteoconductive pellets and/or granules according to the invention.

According to a further aspect of the invention, there is provided a method of treatment for bone repair or replacement, the method comprising the implantation of the osteoconductive pellets and/or granules according to the invention, or the composition according to the invention, into a subject in need thereof.

According to a further aspect of the invention, there is provided the osteoconductive pellets and/or granules according to the invention, or the composition according to the invention, for use as a medicament.

According to a further aspect of the invention, there is provided the osteoconductive pellets and/or granules according to the invention, or the composition according to the invention, for use in bone repair or replacement in a subject.

### BRIEF DESCRIPTION OF DRAWINGS

FIG. 1: Set-up of the HME equipment for the production of Certoss™ Conduit.

FIG. 2: Sintering conditions investigated.

FIG. 3: Certoss™ Conduit having [A] 50% β-TCP, [B] 70% β-TCP (HOL 001) and [C] 80% β-TCP (HOL 005) pre and post sintering.

FIG. 4: Surface topography of Certoss™ Conduit batch HOL 005 [A]×500 [B] ×2000.

FIG. 5: [A] HOL 002 without a pre-feedstock [B] HOL 005 with a pre-feedstock.

FIG. 6: Light microscope images of pellets formed under different sintering conditions.

FIG. 7: SEM images (×2000) showing surface of Certoss™ Conduit pellets formed under different sintering conditions.

FIG. 8: SEM image of Certoss™ Conduit batch HOL 007 which did not sinter.

FIG. 9: [A] is batch HOL 008 pre-sinter [B] is batch HOL 008 post-sinter [C] is the sizing data for HOL 008.

FIG. 10: SEM of the surface of batch HOL 008 manufactured with milled (<4 micron) β-TCP.

FIG. 11: The surface of a Certoss™ Conduit pellet [A] pre-HTT and [B] post HTT with α-TCP supplied by Himed and milled to <150 μm.

FIG. 12: [A] the surface of a Certoss™ Conduit pellet pre-HTT [B] the surface of a Certoss™ Conduit pellet post HTT with α-TCP supplied by Plasma Biotal [C] the surface of a Certoss™ Granule post HTT with α-TCP supplied by Plasma Biotal [D] the surface of a Certoss™ Granule post HTT with α-TCP supplied by Himed and milled to <50 micron.

FIG. 13: Demonstration of wicking of a liquid through a Certoss™ Conduit pellet.

FIG. 14: Protein adsorption to three different batches of Certoss™ Conduit pellets.

FIG. 15: Schematic of the Certoss™ Conduit manufacturing process.

Comparative FIG. 16: Surface of a BCP granule [A] before and [B] after HTT at 180° C. for 2 hours.

FIG. 17: SEM images at ×500 and ×2000 showing the surface of β-TCP granules that are [A] untreated, [B] HTT 100° C. 24 hrs and [C] HTT 100° C. 24 hrs plus α-TCP.

FIG. 18: Effect of HTT time on surface needle growth at 180° C.

Comparative FIG. 19: Needle size analysis for MagnetOs™. A: Needle length; B: Needle diameter.

FIG. 20: Effect of HTT temperature on surface needle growth.

FIG. 21: Needle size analysis for [A] β-TCP+α-TCP (milled and sieved to <150 μm) HTT 100° C. 20 hrs [B] β-TCP+α-TCP (milled and sieved to <50 μm) HTT 100° C. for 24 hrs.

FIG. 22: Certoss™ Granules having HTT with differing amounts of α-TCP present.

FIG. 23: Adsorption of protein to HTT β-TCP, untreated β-TCP and untreated β-TCP from a different supplier.

FIG. 24: Effect of pH during hydrothermal treatments on needle size on the surface of β-TCP. A: No pH adjustment (pH 6 after HTT), Length: 4.7 μm [range 1.7 μm-12.7 μm], Diameter: 0.46 μm [range 0.05 μm-2.4 μm]; B: pH adjusted to 11 with NaOH; length: 1.4 μm [range 4.8 μm-1.4 μm], Diameter: 0.1 μm [range 0.03 μm-0.3 μm].

FIG. 25: Presto Blue data showing metabolic activity of C2C12 cells attached to Certoss™ Granules having surface needles of different sizes.

FIG. 26: SEM images showing attachment of C2C12 cells to the surface of HTT β-TCP at [A]×100, [B]×500 and [C]×2000. Image [D] shows the attachment of C2C12 to untreated β-TCP.

FIG. 27: Schematic of the Certoss™ Granules manufacturing process.

FIG. 28: SEM imaging ×2000 for seven different ceramic samples. A—Untreated b-TCP (CaP); B—Osteophil; C—MagnetOs™; D—Reprobone™; E—Untreated b-TCP HIMED: F—Certoss™ Granules (Batch 41); G—Certoss™ Granules (Batch 60).

FIG. 29: Protein adsorption to a range of ceramic granules and predicates

FIG. 30: SEM imaging of the surface of the osteoconductive pellets of the invention with submicron grains and pores. The solid white bars indicate the scale of 1 micron.

Comparative FIG. 31: SEM image showing the morphology of untreated, M1-treated and M2-treated macrophage cultures from Heinrich et al. PLOS One. 2017 Aug. 17; 12 (8): e0183572. doi: 10.1371/journal.pone.0183572. eCollection 2017. A: Unstimulated macrophages (MO-Day 7). Small and roundish morphology, lacking cytoplasmic extensions. B: M1-treated macrophages (day 7). Enlarged amoeboid cell shape with roundish cell bodies and numerous delicate cytoplasmic extensions on the cellular surface. C and D: M2-treated macrophage cultures (day 7). Demonstrate a marked heterogeneity with two dominating cell types. Large “spindeloid” macrophages with an elongated cell body and cytoplasmic extensions on the apical ends of the cell bodies (C). Second, in M2-cultures, numerous multinucleated giant cells (MNGs) with abundant cytoplasmic projections on the cellular surface are present (D).

FIG. 32: SEM images of the surface of the osteoconductive pellets of the invention with a macrophage exhibiting the same characteristics as the M2-treated macrophage of FIG. 31C. A: ×1000 and B: ×2000.

FIG. 33: Images of conduits after the sintering conditions of Table 10.

FIG. 34: Yield strength of conduits.

FIG. 35: Representative graphs of Yield strength between sintered conduits group where in a. Temperature is the dependent variable and in b. heating rate is the dependent variable.

FIG. 36: Sintering time has no impact on conduits Young's modulus at each sintered conditions stated in the graphs.

FIG. 37: Representative graphs of Young's Modulus between sintered conduits group where in a. Temperature is the dependent variable and in b. heating rate is the dependent variable.

FIG. 38: Toluidine blue staining of macrophages differentiated onto ceramics granules, conduits and predicates. A lot of cells on HG, UC and MG. Cells were situated in the concavities of the granules.

FIG. 39: Representative SEM images of differentiated macrophages on Certoss™ conduits, granules, and predicates.

FIG. 40: (a), (b) Pro-inflammatory cytokines TNFα and IL-1β expressed in differentiated macrophages on ceramic materials at D4. (c) Anti-inflammatory cytokine CCL18 expressed in differentiated macrophages on ceramic materials at D4.

FIG. 41: DNA concentration of differentiated macrophages retrieved from ceramics materials.

FIG. 42: Example Faxitron radiographs with the spine (left) and after muscle harvest (right) at 6 weeks. Differentiation of new bone and residual material is not possible based on this endpoint. No adverse reactions were noted. All materials were present.

FIG. 43: Example of Micro-CT screen shot for group 2 at 12 weeks (3715 LM3).

New bone formation was noted on examination of the Micro-CT data (arrow).

FIG. 44: Example of Micro-CT screen shot for group 4 at 12 weeks (3715 LL4). New bone formation was noted on examination of the Micro-CT data (arrow).

FIG. 45: Example of Micro-CT screen shot for group 5 at 12 weeks (3715 RM2). New bone formation was noted on examination of the Micro-CT data (arrow).

FIG. 46: Example of paraffin histology at 12 weeks for Group 2-(3715 LM3). The implanted material has begun to resorb but still present (star). New bone (black arrow) was noted on and around the material. No inflammatory cells are present.

FIG. 47: Example of paraffin histology at 12 weeks for Group 4-(3715 LL4). The implanted material has begun to resorb but still present (star). New bone (black arrow) was noted on and around the material. No inflammatory cells are present.

FIG. 48: Example of paraffin histology at 12 weeks for Group 5-(3715 LL4). The implanted material has begun to resorb but still present (star). New bone (black arrow) was noted on and around the material. No inflammatory cells are present.

### DETAILED DESCRIPTION

According to a first aspect of the invention there is provided a method of producing osteoconductive pellets, the method comprising:
(i) providing powdered calcium phosphate material mixed with polymer particles to form a feedstock;
(ii) shaping the feedstock to form pellets,
(iii) sintering the pellets, wherein the sintering comprises:
(a) a first stage heating at a temperature and time that is sufficient to burn off substantially all of the polymer and leave calcium phosphate material pellets; and
(b) a second stage heating at a higher temperature of between 950° C. and 1150° C. to sinter the calcium phosphate material pellets, thereby forming osteoconductive pellets with surface topography in the form of 0.2 to 20 micron grains; and optionally
(c) applying a hydrothermal treatment to the osteoconductive pellets to enhance their surface topography.

According to a second aspect of the present invention, there is provided a method of producing osteoconductive β-TCP granules; the method comprising:
(i) providing β-TCP granules;
(ii) adding α-TCP particles and water to the β-TCP granules to form seeded β-TCP granules; and
(iii) applying a hydrothermal treatment to the seeded β-TCP granules, wherein the hydrothermal treatment comprises heating to a temperature of between about 90° C. and 120° C. in the presence of water to enhance the surface topography of the β-TCP granules, thereby forming the osteoconductive β-TCP granules.

### Hydrothermal Treatment (HTT)

In one embodiment, the method of producing osteoconductive pellets further comprises a hydrothermal treatment of the osteoconductive pellets to enhance their surface topography. Preferably, when the powdered calcium phosphate material particle size used in the method is greater than 0.02 microns, a hydrothermal treatment of the osteoconductive pellets is provided to enhance their surface topography.

Enhancing the surface topography of the osteoconductive pellets and/or granules may comprise the formation of needle structures on the surface of the pellets and/or granules. The needle structures may comprise or consist of needle projections from the surface of the osteoconductive pellets and/or granules. The needles may contain calcium phosphate. The needle projections may have a high aspect ratio, e.g. longer than they are wide, preferably at least 5 to 10-fold longer than they are wide. The needle projections may be at least 2 microns in length, on average. In another embodiment, the needle projections may be at least 4 microns in length, on average. The needle projections may be about 1-20 microns in length, on average. The needle projections may be less than 1 micron in width, on average. In one embodiment, the needle projections are between 4 and 15 microns in length and between 0.3 and 2 microns in width, on average. In a preferred embodiment, the needle projections are between 4 and 10 microns in length and between 0.3 and 1 micron in width, on average. In another embodiment, the needle projections are between 4 and 9 microns in length and between 0.4 and 1 micron in width, on average. The length and width of the needle projections may be measured by scanning electron microscopy (SEM) to image the surface of the pellet or granule, including the needle projections. Software such as Image-J software may then be used to process and analyse the image produced by SEM. The average length or width discussed here is the mean length or mean width.

The method may further comprise the addition of α-TCP particles. Preferably, in an embodiment where the method includes a hydrothermal treatment to enhance surface topography, α-TCP is provided. α-TCP (α-tricalcium phosphate) may be provided in powdered form onto the surface of the osteoconductive pellets and/or granules prior to hydrothermal treatment. The α-TCP particles may be added at a ratio of 1:5 to 1:10 w/w α-TCP particles to the osteoconductive pellets and/or granules. The α-TCP particles may be added to the osteoconductive pellets and/or granules with water. At least 100 mg of α-TCP particles may be provided per 1 g of the osteoconductive pellets and/or granules. In another embodiment, at least 25 mg of α-TCP particles may be provided per 1 g of the osteoconductive pellets and/or granules. In another embodiment, at least 200 mg of α-TCP particles may be provided per 1 g of the osteoconductive pellets and/or granules. In another embodiment, between 100 mg and 200 mg of α-TCP particles may be provided per 1 g of the osteoconductive pellets and/or granules. The amount of water may be 20-200 ml per 1 g of the osteoconductive pellets and/or granules. In another embodiment, the amount of water may be 20-100 ml per 1 g of the osteoconductive pellets and/or granules. In another embodiment, the amount of water may be 30-100 ml per 1 g of the osteoconductive pellets and/or granules. In another embodiment, the amount of water may be 40-60 ml per 1 g of the osteoconductive pellets and/or granules. The amount of water may be about 50 ml per 1 g of the osteoconductive pellets and/or granules. In another embodiment, at least 100 mg of α-TCP particles and about 30-100 ml water may be provided per 1 g of the osteoconductive pellets and/or granules. In another embodiment, at least 100 mg of α-TCP particles and about 40-60 ml water may be provided per 1 g of the osteoconductive pellets and/or granules. In another embodiment, about 100 mg to 200 mg of α-TCP particles and about 40-60 ml water may be provided per 1 g of the osteoconductive pellets and/or granules. The α-TCP particles may be added to the osteoconductive pellets and/or granules, optionally with water, together with agitation (e.g. physical mixing/swirling).

The α-TCP particles may be less than 200 μm in size. Preferably, the α-TCP particles may be less than 150 μm in size. In another embodiment, the α-TCP particles may be less than 100 μm in size. In another embodiment, the α-TCP particles may be less than 50 μm in size. The α-TCP particles may be greater than 30, 40 or 45 μm in size. In one embodiment, the α-TCP particles are between 10 μm and 150 μm in size. In another embodiment, the α-TCP particles are between 20 μm and 150 μm in size. In another embodiment, the α-TCP particles are between 40 μm and 150 μm in size. In another embodiment, the α-TCP particles are between 50 μm and 150 μm in size. In one embodiment, the α-TCP particles may be about 20 μm, such as 20.3 μm. The α-TCP particles may be provided by milling and/or sieving α-TCP particles, such as 500-1000 micron particles, to less than 150 μm. The size of the α-TCP particles may be achieved, and also measured, by milling and/or sieving, preferably sieving, the α-TCP particles. The sieve may be selected so as to provide a particle size of less than or equal to the specified size of the openings of the sieve membrane or mesh. The size of the α-TCP particles may also be measured using a particle size analyser, preferably a light scattering particle size analyser, for example, ANALYSETTE 22 NEXT Nano from Fritsch.

In a preferred embodiment, the α-TCP particles are soluble in water.

The hydrothermal treatment may comprise heating (e.g. 90-120° C.) in the presence of water for a period of time sufficient to enhance the surface topography of the pellets and/or granules. In one embodiment, the hydrothermal treatment is at a temperature of between about 100° C. and 120° C. In another embodiment, the hydrothermal treatment is at a temperature of between about 100° C. and 115° C. In one embodiment, the hydrothermal treatment is at a temperature of about 115° C. The surface topography may be enhanced when the surface is at least partially covered or substantially covered in needles as described herein. For example, the surface topography may be enhanced when the surface comprises a plurality of needles having dimensions of about 0.1-1 μm wide and about 4-10 μm long. This period may be at least 5 hours, at least 10 hours, or at least 15 hours. In one embodiment, the hydrothermal treatment period is about 5 to 24 hours, or more. In one embodiment, the hydrothermal treatment period is about 5 to 30 hours, or more. More preferably, 10 to 30 hours, or more. Still more preferably, 20 to 30 hours, or more. The hydrothermal treatment may be for no more than 48 hours. In another embodiment, the hydrothermal treatment may be for no more than 60 hours. In a preferred embodiment the hydrothermal treatment is for a period of 24 hours.

Advantageously, the greater hydrothermal treatment time, such as over 20 hours, provides longer needle structures on the surface.

The hydrothermal treatment may be under a pressurised environment. The hydrothermal treatment may be at a pressure of between 0.5 MPa and 3 MPa. In another embodiment, the hydrothermal treatment may be at a pressure of between 1 MPa and 3 MPa. In another embodiment, the hydrothermal treatment may be at a pressure of between 2 MPa and 3 MPa. The pressure of the hydrothermal treatment may be set by the apparatus used to perform the hydrothermal treatment. Alternatively, the pressure of the hydrothermal treatment may be measured as a function of volume and temperature.

The hydrothermal treatment may be at a temperature of about 100° C. and for at least 5 hours, 10 hours or 15 hours. In another embodiment, the hydrothermal treatment may be at a temperature of about 90-120° C. and for at least 5 hours, 10 hours or 15 hours. The hydrothermal treatment may be for a period of no more than 24 hours. The hydrothermal treatment may be for a period of 20 to 24 hours, or more. The hydrothermal treatment may be for a period of 20 to 30 hours. In a preferred embodiment, the hydrothermal treatment is for a period of 20 to 24 hours. In a further preferred embodiment, the hydrothermal treatment is for a period of 20 to 24 hours at about 100° C. The hydrothermal treatment may be at a temperature of between about 90° C. and 110° C. In another embodiment, the hydrothermal treatment may be at a temperature of between about 90° C. and 105° C. In another embodiment, the hydrothermal treatment may be at a temperature of between about 95° C. and 110° C. In one embodiment the hydrothermal treatment is at a temperature of about 115° C. for a period of about 24 hours. In one embodiment the hydrothermal treatment is at a temperature of about 115° C. for a period of about 20-30 hours,

Advantageously, such HTT temperature and times are optimised for a desirable needle formation that is osteoconductive, for example needles of 0.4×8.8 μm (width×length).

The hydrothermal treatment may be at a temperature of about 100° C. and for at least 5 hours, 10 hours or 15 hours and at a pressure of between 0.5 MPa and 3 MPa. In another embodiment, the hydrothermal treatment may be at a temperature of about 90-120° C. and for at least 5 hours, 10 hours or 15 hours and at a pressure of between 0.5 MPa and 3 MPa. The hydrothermal treatment may be for a period of no more than 24 hours and at a pressure of between 0.5 MPa and 3 MPa. The hydrothermal treatment may be for a period of 20 to 24 hours, or more and at a pressure of between 0.5 MPa and 3 MPa. The hydrothermal treatment may be for a period of 20 to 30 hours and at a pressure of between 0.5 MPa and 3 MPa. In a preferred embodiment, the hydrothermal treatment is for a period of 20 to 24 hours and at a pressure of between 0.5 MPa and 3 MPa. In a further preferred embodiment, the hydrothermal treatment is for a period of 20 to 24 hours at about 100° C. and at a pressure of between 0.5 MPa and 3 MPa. The hydrothermal treatment may be at a temperature of between about 90° C. and 110° C. and at a pressure of between 0.5 MPa and 3 MPa. In another embodiment, the hydrothermal treatment may be at a temperature of between about 90° C. and 105° C. and at a pressure of between 0.5 MPa and 3 MPa. In another embodiment, the hydrothermal treatment may be at a temperature of between about 95° C. and 110° C. and at a pressure of between 0.5 MPa and 3 MPa. In one embodiment the hydrothermal treatment is at a temperature of about 115° C. for a period of about 24 hours and at a pressure of between 0.5 MPa and 3 MPa. In one embodiment the hydrothermal treatment is at a temperature of about 115° C. for a period of about 20-30 hours and at a pressure of between 0.5 MPa and 3 MPa.

A drying step may be provided following the hydrothermal treatment. The skilled person will recognise that a drying step may provide sufficient time and temperature to dry the osteoconductive pellets and/or granules, and preferably without further altering their structure. For example, the drying step may be at less than 100° C., preferably less than 80° C., and may be at about 60° C., or less. In one embodiment, the osteoconductive pellets and/or granules may be left to dry at ambient temperature.

The hydrothermal treatment may be at neutral pH. The hydrothermal treatment may be at a pH of less than 10. The hydrothermal treatment may be at about pH 6 to pH7.

### Hardening Step

In an embodiment, where the osteoconductive pellets and/or granules are hydrothermally treated, the method may further comprise a hardening step. The hardening step may comprise dry-heating the osteoconductive pellets and/or granules, for example in a furnace. The hardening step may comprise heating to about 500° C. to 750° C. for at least 3 hours. The hardening step may comprise heating to about 500° C. to 750° C. for 3-12 hours. In a preferred embodiment, the hardening step may comprise heating to about 500° C. for at least 3 hours. The heating may be no more than 12 hours at 650-750° C.

The heating may be ramped to temperature at a rate that is suitable for the furnace and any container or platform holding the pellets, for example a crucible. The heating may be ramped to temperature at a rate of less than 200° C. per hour. The heating may be ramped to temperature at about 2° C./minute.

### The Polymer Particles (to Form the Feedstock)

The polymer of the polymer particles may be a synthetic polymer, such as a thermoplastic polymer. The polymer may comprise polymerized ethylene. In one embodiment, the polymer is any polymer with a melting point between about 25° C. and 400° C. In another embodiment, the polymer is any polymer with a melting point between 40° C. and 400° C. The polymer may comprise a polymer selected from polyethylene, acetal, polytetrafluoroethylene, polybutyleneterephthlate, nylon, such as nylon 6, 6/6, 6/10, 11 and/or 12, polyetheretherketone, polypropylene, and polyvinylidenefluoride; or copolymers thereof. The polymer particles may comprise or consist of ethylene vinyl acetate (EVA).

Preferably the polymer particles are less than 1 mm. The polymer particles may be provided at a particle size that is suitable to enable the feedstock to mix and be extruded, such as hot melt extruded, or 3D printed. The polymer particles may be between 0.01 mm and 1 mm in size, on average. In another embodiment, the polymer particles may be between 0.1 mm and 1 mm in size, on average. In another embodiment, the polymer particles may be between 0.2 mm and 0.5 mm in size, on average.

The polymer particles may be provided at a required particle size by milling. Reference to particle size is understood to be the mean widest diameter in a population of particles. The particle size may be measured by milling and/or sieving, preferably sieving, the polymer particles. The sieve may be selected so as to provide a particle size of less than or equal to the specified size of the openings of the sieve membrane or mesh.

### The Powdered Calcium Phosphate Material (to Form the Feedstock)

The powdered calcium phosphate material may comprise or consist of β-TCP (β-tricalcium phosphate), BCP (biphasic calcium phosphate) or hydroxyapatite. In one embodiment, the powdered calcium phosphate material comprises or consist of β-TCP or BCP. In a preferred embodiment, the powdered calcium phosphate material comprises or consists of β-TCP.

The powdered calcium phosphate material may have a particle size of less than 25 μm. The powdered calcium phosphate material may have a particle size of 0.02-25 μm. Preferably, the powdered calcium phosphate material has a particle size of about 0.02 to 10 μm. In another embodiment, the powdered calcium phosphate material has a particle size of less than about 4 μm. In another embodiment, the powdered calcium phosphate material has a particle size of about 0.02 to 4 μm.

In one embodiment, the powdered calcium phosphate material has a particle size of about 7 μm. Reference to particle size is understood to be the mean widest diameter in a population of particles. The particle size of the powdered calcium phosphate may be measured using a particle size analyser, preferably a light scattering particle size analyser, for example, ANALYSETTE 22 NEXT Nano from Fritsch.

The powdered calcium phosphate material D10 size may be about 0.7-0.9 microns, the D50 size may be about 1.5-2 microns, and the D90 size may be about 2.9-3.9 microns. In another embodiment, the powdered calcium phosphate material D10 size may be about 0.5-1 microns, the D50 size may be about 1-2 microns, and the D90 size may be about 3-5 microns. In another embodiment, the average powdered calcium phosphate material D10 size may be about 0.69 microns, the D50 size may be about 1.49 microns, and the D90 size may be about 2.86 microns. In another embodiment, the average β-TCP powdered calcium phosphate material D10 size may be about 0.87 microns, the D50 size may be about 1.96 microns, and the D90 size may be about 3.95 microns. The D10, D50 and D90 values may be measured using a particle size analyser, preferably a light scattering particle size analyser, for example, ANALYSETTE 22 NeXT Nano from Fritsch. D10 refers to the particle size below which 10% of particles of the powdered calcium phosphate material are found. D50 refers to the particle size below which 50% of the particles of the powdered calcium phosphate material are found. D90 refers to the particle size below which 90% of the particles of the powdered calcium phosphate material are found.

The powdered calcium phosphate material may be provided at a required particle size by milling.

### The Feedstock and the Pelleting Process

In one embodiment, the feedstock is shaped into pellets by extrusion. The extrusion may further comprise pelleting, for example by cutting the extruded feedstock into pellets. In one embodiment, the extrusion is a hot melt extrusion (HME). In another embodiment, the extrusion is in a 3D printing process. In a preferred embodiment, hot melt extrusion is used.

In one embodiment, the feedstock is shaped into pellets by 3D printing of the feedstock into the pellet shape. The 3D printing may layer the feedstock or extrude the feedstock into the pellet shape. For 3D printing, the polymer (e.g. ethylene vinyl acetate (EVA)) may be melted and mixed with the powdered calcium phosphate material to form a granular paste. The paste may be extruded by the printer head and cools when it leaves the nozzle. The shape of the composite may be determined by the movement of the printer head relative to the extruded material.

The powdered calcium phosphate material may be dry mixed with the polymer, such as ethylene vinyl acetate, to create a feedstock for hot melt extrusion.

The feedstock may comprise at least 60% w/w powdered calcium phosphate material. Alternatively, the feedstock may comprise at least 70% w/w powdered calcium phosphate material. In another embodiment, the feedstock may comprise at least 72% w/w powdered calcium phosphate material. In another embodiment, the feedstock may comprise at least 75% w/w powdered calcium phosphate material. In another embodiment, the feedstock may comprise at least 78% w/w powdered calcium phosphate material. In another embodiment, the feedstock may comprise between 60% and 90% w/w powdered calcium phosphate material. In another embodiment, the feedstock may comprise between 70% and 90% w/W powdered calcium phosphate material. In another embodiment, the feedstock may comprise between 75% and 90% w/w powdered calcium phosphate material. In another embodiment, the feedstock may comprise between 75% and 85% w/w powdered calcium phosphate material. In another embodiment, the feedstock may comprise between 78% and 82% w/w powdered calcium phosphate material.

In another embodiment, the feedstock may comprise between 80% and 90% w/W powdered calcium phosphate material. In another embodiment, the feedstock may comprise between 80% and 85% w/w powdered calcium phosphate material. About 72% to 90% w/w powdered calcium phosphate material may be mixed with about 10% to 28% w/w of the polymer particles. About 72% to 90% w/w powdered calcium phosphate material may be mixed with about 10% to 18% w/w of the polymer particles. In a preferred embodiment, the powdered calcium phosphate material is mixed with the polymer particles in a 4:1 ratio w/w (80% and 20%).

In one embodiment, a pre-feedstock is provided, for example for initial loading and hot melt exclusion prior to the feedstock hot melt extrusion. The pre-feedstock may be provided in a sufficient amount to facilitate loading and hot melt exclusion of the feedstock. The pre-feedstock may comprise powdered calcium phosphate material mixed with the polymer particles, wherein the ratio of powdered calcium phosphate material to polymer particles is substantially less than the feedstock ratio. The pre-feedstock may comprise less than 60% (w/w) powdered calcium phosphate material. The pre-feedstock may comprise the powdered calcium phosphate material mixed with the polymer particles in a ratio of about 10-60% powdered calcium phosphate material to 40-90% polymer particles (w/w). In a preferred embodiment, the pre-feedstock may comprise the powdered calcium phosphate material mixed with the polymer particles in a 1:1 ratio (50% to 50%) (w/w).

The provision of a pre-feedstock advantageously prevents the clogging of the hot melt extrusion apparatus by the feedstock that may have a higher powdered calcium phosphate material content. In particular, the hot melt extrusion process can be initiated effectively with a pre-feedstock material which is easier to extrude, and this can be followed by the main feedstock for the desired pellet material characteristics.

The powdered calcium phosphate material of the pre-feedstock may be substantially the same size as the calcium phosphate material of the feedstock. The pre-feedstock may be provided in embodiments wherein the powdered calcium phosphate material particle size is greater than about 0.02 microns. The pre-feedstock may not be provided in embodiments wherein the powdered calcium phosphate material particle size is less than 0.02 microns. The powdered calcium phosphate material of the pre-feedstock may have a particle size of less than 25 μm. The powdered calcium phosphate material of the pre-feedstock may have a particle size of 0.02-25 μm. Preferably, the powdered calcium phosphate material of the pre-feedstock has a particle size of about 0.02 to 10 μm. In another embodiment, the powdered calcium phosphate material of the pre-feedstock has a particle size of less than about 4 μm. In another embodiment, the powdered calcium phosphate material of the pre-feedstock has a particle size of about 0.02 to 4 μm. In one embodiment, the powdered calcium phosphate material has a particle size of about 7 μm. Reference to particle size is understood to be the mean widest diameter in a population of particles. The particle size of the powdered calcium phosphate may be measured using a particle size analyser, preferably a light scattering particle size analyser, for example, ANALYSETTE 22 NeXT Nano from Fritsch.

The pre-feedstock powdered calcium phosphate material D10 size may be about 0.7-0.9 microns, the D50 size may be about 1.5-2 microns, and the D90 size may be about 2.9-3.9 microns. In another embodiment, the pre-feedstock powdered calcium phosphate material D10 size may be about 0.5-1 microns, the D50 size may be about 1-2 microns, and the D90 size may be about 3-5 microns. In another embodiment, the average pre-feedstock powdered calcium phosphate material D10 size may be about 0.69 microns, the D50 size may be about 1.49 microns, and the D90 size may be about 2.86 microns. In another embodiment, the average pre-feedstock powdered calcium phosphate material D10 size may be about 0.87 microns, the D50 size may be about 1.96 microns, and the D90 size may be about 3.95 microns. The D10, D50 and D90 values may be measured using a particle size analyser, preferably a light scattering particle size analyser, for example, ANALYSETTE 22 NeXT Nano from Fritsch. D10 refers to the particle size below which 10% of particles of the powdered calcium phosphate material are found. D50 refers to the particle size below which 50% of the particles of the powdered calcium phosphate material are found. D90 refers to the particle size below which 90% of the particles of the powdered calcium phosphate material are found.

The skilled person may set the hot melt extrusion at an appropriate speed and temperature for extrusion of the feedstock. The hot melt extrusion may be at a temperature that is suitable for hot melt extrusion of the polymer of the polymer particles. The hot melt extrusion may be at a temperature of no more than 210° C. In one embodiment, the hot melt extrusion temperatures are set at about 90° C., 130° C., 130° C., and 130° C. and/or a feed-screw speed may be set at about 15 rpm for the feedstock (optionally an initial higher speed, such as 20 rpm, for the pre-feedstock).

The pelleting of the extruded feedstock may be provided by cutting the extruded feedstock into pellets, for example, as it emerges from the extrusion. The cutting into pellets may be by use of a pelletiser. The pelletiser may be set at about 0.1 to 0.4 m/min pull and about 1-4 mm cut. In one embodiment, the pelletiser is set at about 0.4 m/min pull and about 3 mm cut. The skilled person may adjust the pelletiser pull and cut parameters depending on the required pellet size.

A conveyer may carry the extruded feedstock to a pelletiser. The skilled person may readily adjust the conveyer speed as appropriate for the extrusion and pelletiser speed.

### The Sintering of the Pelleted Powdered Calcium Phosphate Material

The skilled person will recognise that the sintering is a dry-heated process to form a solid mass of material (i.e. the pellets) from the pelleted powdered calcium phosphate material without fully liquifying the calcium phosphate material. i.e. in the absence of sintering the extruded pellet material would crumble to a powder in the absence of the polymer that is burnt off.

In a preferred embodiment, the sintering is in a furnace.

The first stage (a) heating may be at a temperature and time sufficient to burn off substantially all of the polymer, i.e. such that the powdered calcium phosphate material in pellet form remains without the polymer content. In another embodiment, the first stage (a) heating may be at a temperature and time sufficient to burn off at least 90% w/w of the polymer.

The first stage (a) heating at a temperature and time that is sufficient to burn off the polymer and leave calcium phosphate material pellets may not be at a temperature and/or time to cause sintering of the calcium phosphate material (i.e. lower than the sintering temperature, for example in step b). The first stage (a) heating may be at a temperature of at least about 500° C., but not more than 800° C. The first stage (a) heating may be at a temperature of between about 500° C. and about 800° C. for at least 3 hours. In one embodiment, the first stage (a) heating may be at a temperature of between about 500° C. and about 700° C. for at least 3 hours. In another embodiment, the first stage (a) heating may be at a temperature of between about 500° C. and about 600° C. for at least 3 hours. The heating time may be limited to 3, 4, 5, 6, 7, 8, 9 or 10 hours. The heating time may be limited to 3, 4, 5, 6, 7, 8, 9 or 10 hours at between about 500° C. and about 800° C. The heating time may be no more than 24 hours. In one embodiment, the first stage (a) heating may be at a temperature of between about 500° C. and about 600° C. for about 3-6 hours.

The temperature may be ramped up at a rate that is suitable for the furnace and any container or surface holding the pellets. The temperature may be ramped up at a rate of less than 200° C. per hour. The temperature may be ramped up at a rate of about 2° C./minute.

The second stage (b) heating may be at a temperature and time sufficient time to sinter the calcium phosphate material pellets. The second stage (b) heating may be for a period of at least 1 hour, preferably 1-12 hours. The second stage (b) heating may be for a period of at least 6 hours, preferably 6-12 hours. In another embodiment, the second stage (b) heating may be for a period of 6-10 hours. In one embodiment, the second stage (b) heating may be for a period of about 1-10 hours. The second stage (b) heating may be for a period of about 6 hours.

The second stage (b) heating may not exceed 1150° C. In another embodiment, the second stage (b) heating may not exceed 1100° C. The second stage (b) heating may be at a temperature of between 950° C. and 1100° C. Alternatively, the second stage (b) heating may be at a temperature of between 950° C. and 1150° C. The second stage (b) heating may be at a temperature of between 1000° C. and 1100° C. In another embodiment, the second stage (b) heating may be at a temperature of between 1000° C. and 1080° C. The second stage (b) heating may be at a temperature of less than 1050° C. In one embodiment, the second stage (b) heating may be at a temperature of between 950° C. and 1050° C.

The second stage (b) heating may not exceed 1150° C. and is for 6-12 hours. In another embodiment, the second stage (b) heating may not exceed 1100° C. and is for 6-12 hours. The second stage (b) heating may be at a temperature of between 950° C. and 1100° C. and is for 6-12 hours. Alternatively, the second stage (b) heating may be at a temperature of between 950° C. and 1100° C. and is for 6-12 hours. The second stage (b) heating may be at a temperature of between 1000° C. and 1100° C. and is for 6-12 hours. In another embodiment, the second stage (b) heating may be at a temperature of between 1000° C. and 1080° C. and is for 6-12 hours.

In another embodiment, the second stage (b) heating may not exceed 1150° C. and is for 6-10 hours. In another embodiment, the second stage (b) heating may not exceed 1100° C. and is for 6-10 hours. In another embodiment, the second stage (b) heating may be at a temperature of between 950° C. and 1100° C. and is for 6-10 hours. Alternatively, the second stage (b) heating may be at a temperature of between 950° C. and 1100° C. and is for 6-10 hours. In another embodiment, the second stage (b) heating may be at a temperature of between 1000° C. and 1100° C. and is for 6-10 hours. In another embodiment, the second stage (b) heating may be at a temperature of between 1000° C. and 1080° C. and is for 6-10 hours.

In one embodiment, the second stage (b) temperature is about 1050° C. for at least 6 hrs. In a preferred embodiment, the second stage (b) temperature is about 1050° C. for 6-12 hours. In an alternative embodiment, the second stage (b) temperature is about 1050° C. for 6-10 hours. In a preferred embodiment, the second stage (b) temperature is about 1050° C. for less than 10 hours, such as 1 to 10 hours.

The transition from the first stage (a) to the second stage (b) of the sintering process may be a stepped increase in temperature or a gradual increase. In one embodiment, the first and second stages of the sintering process comprises or consists of two distinct heating stages (e.g. a ramp up in temperature and hold, and another ramp up in the temperature and hold). In another embodiment, the first and second stages of the sintering process comprises a gradual increase (ramp up) of the temperature, or multiple steps up in temperature. The temperature increase (ramp up) may be varied, for example a slower rate of increase in temperature during the first stage and a higher rate of increase for the second stage of the sintering process. The skilled person will recognise that there is a polymer burn off stage (the first stage) and a sintering stage (the second stage) and may adjust the rate(s) of temperature increase accordingly to achieve the required temperatures and times for these stages.

In one embodiment, the heating rate during the sintering process is less than 10° C./min. In another embodiment, the heating rate during the sintering process is less than 9° C./min. In another embodiment, the heating rate during the sintering process is less than 8° C./min. In another embodiment, the heating rate during the sintering process is less than 5° C./min. In another embodiment, the heating rate during the sintering process is about 2° C./min or less. The heating rate may be no more than 10° C./min. In one embodiment, the heating rate may be no more than 10° C./min, and the second stage (b) heating may be at a temperature of between 950° C. and 1050° C. and is for 1-10 hours. In one embodiment, the method comprises a 6-10 hours sintering time, heating rate <10° C./min and a sintering temperature<or equal to 1050° C. In another embodiment, the method comprises a 6-10 hours sintering time, heating rate 2° C./min and a sintering temperature<or equal to 1050° C. In another embodiment, the method comprises a 1-10 hours sintering time, heating rate 2° C./min and a sintering temperature<or equal to 1050° C.

Advantageously, the microporosity within each pellet is achieved by the optimised sintering of the calcium phosphate material pellet. For example, if the sintering temperature is too high, the calcium phosphate material liquidates too much and reduces the microporosity.

### The Osteoconductive Pellet Characteristics

The osteoconductive pellet may comprise at least 90% w/w calcium phosphate material, such as β-TCP. In another embodiment, the osteoconductive pellet comprise at least 95% w/w calcium phosphate material, such as β-TCP. In another embodiment, the osteoconductive pellet comprise at least 98% w/w calcium phosphate material, such as β-TCP. In another embodiment, the osteoconductive pellet comprise at least 99% w/w calcium phosphate material, such as β-TCP. In another embodiment, the osteoconductive pellet consists of 100% w/w calcium phosphate material, such as β-TCP.

The osteoconductive pellet may be osteoinstructive and/or osteoinductive.

The osteoconductive pellets may comprise a concave surface, for example a concave surface within a hollow or recess. Additionally or alternatively, the concave surface may be provided in the form of one or more indents, channels or grooves on the outside surface of the osteoconductive pellets. In an embodiment wherein the osteoconductive pellets have a concave surface and are cylindrical or tubular in shape, the osteoconductive pellet may comprise a cross-section that is substantially in the shape of a cog. In an embodiment wherein the osteoconductive pellets are tubular in shape, they may comprise concave surfaces on the inside of the tube, and one or more concave surfaces recessed into the outer surface of the tube. In a preferred embodiment according to the first aspect of the invention, the osteoconductive pellets may be hollow pellets. In particular, pellets having a hollow structure. The hollow pellet structure may be formed during the extrusion and pelleting step ii). In a preferred embodiment, the pellets formed following extrusion and pelleting step ii) are hollow pellets. In an alternative embodiment, the pellets are solid pellets (i.e. having no lumen/hollow), although they may have microporosity.

The “hollow” may otherwise be termed a “lumen”. The hollow may be considered by the skilled person to be a single continuous macropore.

The hollow pellets may comprise an open hollow. For example, a hollow in a structure that is open at at least one end or side, i.e. not a hollow that is completely enclosed within the structure of the pellet. For example, the hollow pellets may have a tubular structure, with the hollow extending therethrough. The hollow tube may be open at one end or more preferably at both ends of the pellet structure. The invention also envisages a hollow in the form of a channel running through a pellet structure, whereby the channel may be open substantially along its length, i.e. as an alternative to a hollow tube structure having a generally O-shaped hollow cross section the hollow pellets may comprise a C-shaped or U-shaped cross-section such that the hollow channel is open substantially along it length. In one embodiment, the hollow pellets are tubular in structure and open at both ends. The hollow pellets may comprise substantially parallel walls (for example in the sense that opposite walls of a tube are generally parallel to each other). The hollow pellets may not comprise or consist of hollow microspheres (e.g. substantially spherical particles with a substantially hollow core). The hollow pellets may not comprise or consist of a substantially spherical structure.

The hollow pellets may be tubular. In one embodiment, the hollow pellets are tubular with a substantially circular cross-section. Preferably, the osteoconductive pellets (hollow or non-hollow) are cylindrical in shape. In one embodiment wherein the hollow pellets are tubular, the outer surface of the hollow pellets may be substantially circular in cross-section and the inner surface of the hollow pellets may be substantially circular in cross-section. In another embodiment, the cross-sectional shape of the outer surface may be different to the cross-sectional shape of the inner surface. For example, the outer surface may be circular in cross-section, and the inner surface may be square in cross-section, or vice versa. Where the hollow pellets are formed by extrusion, the cross-sectional shape of the inner and outer surfaces of the tube-like structure may be determined by the shape of the extrusion die.

The pellets, such as hollow pellets, may have a length that is equal to or greater than their diameter. In one embodiment, the length is greater than the diameter. The length of the pellets may be uniform in the composition of pellets or a population of pellets in a composition may be irregular in length relative to each other. The pellets may have an aspect ratio of 0.75 to 3 (length to width). In another embodiment, the pellets may have an aspect ratio of 0.75 to 2.2 (length to width). In another embodiment, the pellets may have an aspect ratio of 1.2 to 3 (length to width). In another embodiment, the pellets may have an aspect ratio of 1.2 to 2.2 (length to width). In another embodiment, the pellets have an aspect ratio of 1.4 to 1.2 (length to width). In another embodiment, the pellets may have an aspect ratio of 1.2 to 1.9 (length to width). Alternatively, the pellets may have an aspect ratio of 1.4 to 1.9 (length to width). The pellets may have an aspect ratio of 0.75 to 1 (length to width). In a preferred embodiment, the pellets may have an aspect ratio of 0.9 to 1 (length to width). The width of the pellet may be the diameter where the pellet is tubular, tubular with a substantially circular cross-section, or cylindrical in shape.

Advantageously, the aspect ratio of the pellet is optimised for reduced packing density. In particular, at an aspect ratio of about 1 (length to width), the packing density of the pellets is minimised and therefore maximizes the inter-pellet, interconnected macroporosity through random packing of the pellets together. The interconnected porosity is well understood to be important in bone healing as it allows for the flow of nutrients into the core of the graft site, removal of waste and new blood vessel formation.

The pellets may be between 2 mm and 4 mm in length. The hollow pellets may have a size in their longest dimension of between about 2 mm and about 4 mm. In one embodiment, the pellets are between 2.6 and 3.7 mm in length. The length of each pellet may be measured using callipers.

The pellets may have a diameter of between 2 and 4 mm. In one embodiment, the pellets are between 2.6 and 3.7 mm in diameter. The pellets may have a diameter of between 1.9 and 2.5 mm. In another embodiment, the pellets have a diameter of between 1.95 and 2.49 mm. In another embodiment, the pellets are between 2.6 and 3.7 mm in length and have a diameter of between 1.95 and 2.49 mm. The pellets may have a diameter of between 2 and 4 mm in diameter and a length of between 2 and 4 mm. In one embodiment, the pellets are between 2.6 and 3.7 mm in diameter and between 2.6 and 3.7 mm in length. The length and diameter of each pellet may be measured using callipers.

The internal/lumen diameter of the hollow pellets (i.e. the distance between opposing inner surfaces (e.g. internal diameter of a tube) may be between about 0.3 mm and 0.6 mm. In one embodiment, the internal/lumen diameter of the hollow pellets may be between about 0.29 mm and 0.57 mm. The internal/lumen diameter of the hollow pellets may be about 0.3 mm to 0.5 mm on average. The internal/lumen diameter of the hollow pellets may be about 0.43 mm on average. The internal/lumen diameter of each hollow pellet may be measured using callipers.

The thickness of the walls of the hollow pellets may be at least about 0.2 mm. In one embodiment, the thickness of the walls of the hollow pellets may be between about 0.2 mm and 1 mm. In another embodiment, the thickness of the walls of the hollow pellets may be between about 0.2 mm and 0.86 mm. In another embodiment, the thickness of the walls of the hollow pellets may be between about 0.55 mm and 0.86 mm. In another embodiment, the thickness of the walls of the hollow pellets may be between about 0.6 mm and 0.9 mm. In another embodiment, the thickness of the walls of the hollow pellets may be between about 0.5 mm and 1 mm. In another embodiment, the thickness of the walls of the hollow pellets may be about 0.6 to 0.8 mm on average. In another embodiment, the thickness of the walls of the hollow pellets may be about 0.7 mm on average. The thickness of the walls of each hollow pellet may be measured using callipers.

In one embodiment, the hollow (or otherwise “lumen”) of the hollow pellets may be at least 10% of the volume of the hollow pellets. In another embodiment, the hollow of the hollow pellets may be at least 20% of the volume of the hollow pellets. In another embodiment, the hollow of the hollow pellets may be at least 30% of the volume of the hollow pellets. In another embodiment, the hollow of the hollow pellets may be at least 40% of the volume of the hollow pellets. In another embodiment, the hollow of the hollow pellets may be at least 50% of the volume of the hollow pellets. The volume of the hollow (lumen) may be calculated by mathematical formula V=π(R²−r²) h, where R is the radius of the hollow pellet, r is the radius of the hollow (lumen), and h is the length of the hollow pellet. R, r and h may be measured using callipers.

The size, length, diameter, volume or thickness of the hollow pellets, or features thereof, may refer to the average (mean) size of a population of hollow pellets. In one embodiment, the size, length, diameter, volume or thickness of the hollow pellets, or features thereof, may refer to the largest size, length, diameter, volume or thickness of the hollow pellet.

Macroporosity may be achieved by open lumens within the osteoconductive pellets and by the random packing of pellets to create interconnected macropores between the pellets. The degree of interconnected macropores are a consequence of the aspect ratio of the pellets. Macropores are advantageous for cell infiltration and tissue growth, and microporosity is advantageous for mass transfer.

### The Osteoconductive β-TCP Granule Characteristics

The β-TCP granules (e.g. to generate Certoss™ Granules) may be porous. In one embodiment, the β-TCP granules have microporosity.

The β-TCP granules (e.g. prior to the hydrothermal treatment) may be about 1-2 mm in size. The size may be the average size in a population of β-TCP granules may be. The resulting osteoconductive granules of the invention may be the same size as provided prior to the hydrothermal treatment.

The osteoconductive granules may be 0.5 to 5 mm in size. In another embodiment, the osteoconductive granules may be 1 to 2 mm in size. The size of the osteoconductive granules is taken to be their maximum dimension.

### Porosity

The osteoconductive pellets and/or granules may be porous, such as microporous. In a preferred embodiment, the osteoconductive pellets have macroporosity and microporosity. In embodiment wherein the osteoconductive pellets are hollow pellets, the walls of the pellet structure may be porous.

The macroporosity may be provided by the internal/lumen of the hollow pellets, and/or by spaces between pellets that are packed together. The internal diameter of the hollow pellets (i.e. the distance between opposing inner surfaces (e.g. internal diameter of a tube) may be between about 0.3 mm and 0.6 mm. In one embodiment, the internal/lumen diameter of the hollow pellets may be between about 0.29 mm and 0.57 mm. The internal/lumen diameter of the hollow pellets may be about 0.3 mm to 0.5 mm on average. The internal/lumen diameter of the hollow pellets may be about 0.43 mm on average.

The micropores of the pellets and/or granules may have an average diameter of about 1 micron or less. The osteoconductive pellets and/or granules may have pores in the nanometer to micrometre range. The osteoconductive pellets and/or granules may have pores of about 0.1 to about 20 microns, alternatively between about 0.1 and 10 microns. In one embodiment, the osteoconductive pellets and/or granules have pores with an average size of less than 20 microns. In another embodiment, the osteoconductive pellets and/or granules have pores with an average size of less than 10 microns. In another embodiment, the osteoconductive pellets and/or granules have pores with an average size of less than 2 microns. In a preferred embodiment, the osteoconductive pellets and/or granules have pores with an average size of less than 1 micron. In a further preferred embodiment, the osteoconductive pellets and/or granules have pores with an average size of <1 μm to 2 μm.

The osteoconductive pellets may have pores with an average size of <1 μm to 2 μm and a single macropore/lumen.

The osteoconductive pellets and/or granules may have at least about 30%, about 40%, about 50% or more pore volume. In one embodiment, the porosity of the osteoconductive pellets and/or granules may be between 30% and 70%. In another embodiment, the porosity of the osteoconductive pellets and/or granules may be between 40% and 65%. In another embodiment, the porosity of the osteoconductive pellets and/or granules may be between 40% and 60%. In another embodiment, the porosity of the osteoconductive pellets and/or granules may be between 50% and 60%. The osteoconductive pellets and/or granules may have a pore volume of at least 90 mm³ per 300 mm³ of osteoconductive pellet and/or granule material. In another embodiment, the osteoconductive pellets and/or granules may have a pore volume of at least 120 mm³ per 300 mm³ of osteoconductive pellet and/or granule material. In another embodiment, the osteoconductive pellets and/or granules may have a pore volume of at least 150 mm³ per 300 mm³ of osteoconductive pellet and/or granule material.

As the skilled person would appreciate, porosity, pore volume and pore size can be determined using microcomputer tomography (microCT) and/or scanning electron microscopy (SEM), or mercury porosimetry. SEM images the surface of the pellet or granule. Software such as Image-J software may then be used to process and analyse the image produced by SEM.

The pores may be substantially interconnected. In a preferred embodiment, the osteoconductive pellets and/or granules have interconnected microporosity. The osteoconductive pellets and/or granules may have interconnected microporosity such that they are capable of wicking a fluid, such as water. The wicking may allow for the distribution of a fluid throughout the osteoconductive pellet and/or granule material. The wicking may allow for the distribution of a fluid throughout the osteoconductive pellet and/or granule material in 60 seconds or less. Preferably, the wicking may allow for the distribution of a fluid throughout the osteoconductive pellet and/or granule material in 30 seconds or less.

The osteoconductive pellets and/or granules may be capable of protein adsorption. The osteoconductive pellets and/or granules may be capable of protein adsorption of at least 200 μg/ml protein when incubated at 37° C. in culture media containing 10% fetal bovine serum for a period of 24 hours.

The osteoconductive pellets and/or granules may be capable of inducing ectopic bone formation in a tissue, for example in an animal model, such as a sheep. The model may be as described herein (Example 6).

The osteoconductive pellets and/or granules may be for use in a method of treatment of the human or animal body by surgery or therapy or in a diagnostic method practised on the human or animal body. The osteoconductive pellets and/or granules may be for pharmaceutical use or may be for use in cosmetic surgery.

### Enhanced Surface Topography—Grains

The osteoconductive pellets may have an enhanced surface topography comprising grains on their surface. The grains may be less than 20 microns. In one embodiment the grains are <1 to 20 microns. A proportion of grains, or all grains may be <1 to 2 microns, or less than 1 μm. The surface of the osteoconductive pellets may be substantially covered in grains. In one embodiment, at least 80% of the surface of the osteoconductive pellets is covered in grains. In another embodiment, at least 90% of the surface of the osteoconductive pellets is covered in grains. In another embodiment, at least 95% of the surface of the osteoconductive pellets is covered in grains. The size of the grains may be measured, for example using SEM, according to their largest dimension. The size of the grains may be an average (mean) size of a population of grains on the surface. Surface features greater than 20 microns may not be considered a “grain”.

Advantageously, the method of the invention produces pellets that have grains on the surface. Such grains on calcium phosphate based material are known to induce ectopic bone formation. Therefore, they enhance osteoconductivity and osteoinduction and can be termed ‘osteoinstructive’.

### Enhanced Surface Topography—Needles

The osteoconductive pellets and/or granules may have an enhanced surface topography, e.g. enhanced by HTT relative to untreated pellets or granules. The osteoconductive pellets and/or granules may comprise needle structures on their surface. The needle structures may comprise or consist of needle projections of calcium phosphate material from the surface. The needle projections may have a high aspect ratio, e.g. longer than they are wide, preferably at least 5 to 10-fold longer than they are wide. The needle projections may be at least 2 microns in length, on average. In another embodiment, the needle projections may be at least 4 microns in length, on average. The needle projections may be about 1-20 microns in length, on average. The needle projections may be less than 1 micron in width, on average. In one embodiment, the needle projections are between 4 and 15 microns in length and between 0.3 and 2 microns in width, on average. In a preferred embodiment, the needle projections are between 4 and 10 microns in length and between 0.3 and 1 micron in width, on average. In another embodiment, the needle projections are between 4 and 9 microns in length and between 0.4 and 1 micron in width, on average. The length and width of the needle projections may be measured by scanning electron microscopy (SEM) to image the surface of the pellet or granule, including the needle projections. Software such as Image-J software may then be used to process and analyse the image produced by SEM. The average length or width discussed here is the mean length or mean width.

At least 30% of the surface of the osteoconductive pellets and/or granules may be covered in needle structures. In another embodiment, at least 40%, 50% or 60% of the surface of the osteoconductive pellets and/or granules may be covered in needle structures. In one embodiment, at least 70%, 80% or 90% of the surface of the pores of the osteoconductive pellets and/or granules may be covered in needle structures. The % of the surface of the osteoconductive pellets and/or granules covered in needle structures may be measured by scanning electron microscopy (SEM) to image the surface of the pellet or granule, including the needle projections. Software such as Image-J software may then be used to process and analyse the image produced by SEM.

Following HTT treatment, the surface of the pellets may comprise both grains and needles.

### Further Additives

The osteoconductive pellets and/or granules may further comprise one or more active agents. Additionally or alternatively, the osteoconductive pellets and/or granules may comprise cells, such as osteoblast cells and/or chondrocytes. The cells may be any suitable cell that is capable of supporting tissue repair, such as bone.

The active agent(s) and/or cells may be added into the osteoconductive pellets and/or granules following the sintering process, or following the hydrothermal treatment.

The active agent may be a therapeutically, prophylactically or diagnostically active substance. It may be any bioactive agent.

The active agent for delivery may be a drug, a cell, signalling molecule, such as a growth factor, or any other suitable agent. For example, the active agent may comprise amino acids, peptides, proteins, sugars, antibodies, nucleic acid, antibiotics, antimycotics, growth factors, nutrients, enzymes, hormones, steroids, synthetic material, adhesion molecules, colourants/dyes (which may be used for identification), radioisotopes (which may be for X-ray detection and/or monitoring of degradation), and other suitable constituents, or combinations thereof.

Other active agents which may be added include but are not limited to epidermal growth factor, platelet derived growth factor, basic fibroblast growth factor, vascular endothelial growth factor, insulin-like growth factor, nerve growth factor, hepatocyte growth factor, transforming growth factors and other bone morphogenic proteins, cytokines including interferons, interleukins, monocyte chemotactic protein-1 (MCP-1), oestrogen, testosterone, kinases, chemokinases, glucose or other sugars, amino acids, calcification factors, dopamine, amine-rich oligopeptides, such as heparin binding domains found in adhesion proteins such as fibronectin and laminin, other amines, tamoxifen, cis-platin, peptides and certain toxoids. Additionally, drugs (including statins and NSAIDs), hormones, enzymes, nutrients or other therapeutic agents or factors or mixtures thereof may be included. In one embodiment, the active agent comprises bone morphogenic protein, such as BMP-2.

The active agent may comprise nucleic acid, such as DNA, RNA, or plasmid.

In some embodiments, the active agent for is a statin, e.g. simvastatin, atorvastatin, fluvastatin, pravastatin or rosuvastatin. The statin may be simvastatin. Embodiments in which the active agent is a statin are particularly suitable for the treatment of orthopaedic indications, craniomaxillofacial surgery and dentistry.

The active agent may be in an amount effective to have a desired local or systemic physiological or pharmacological effect.

It is possible to use any animal cell with the osteoconductive pellets and/or granules of the invention. Examples of cells which may be used include bone, osteoprogenitor cells, cartilage, muscle, liver, kidney, skin, endothelial, gut, intestinal, cardiovascular, cardiomycotes, chondrocyte, pulmonary, placental, amnionic, chorionic, foetal or stem cells. Where stem cells are used, preferably non-embryonic stem cells are used. The cells may be included for delivery to the site of repair, or they may be included and intended to be retained in the pellet, for example, to encourage colonisation of the pellet.

In one embodiment, the surface of the osteoconductive pellets and/or granules may be treated prior to introducing cells in order to enhance cell attachment. Surface treatments may comprise coating techniques to coat the surfaces of the polymer microparticles with an agent capable of enhancing or facilitating cell attachment. Additionally or alternatively, surface treatments may comprise physical or chemical modifications to the surface of the osteoconductive pellets and/or granules. In surface coating, the osteoconductive pellets and/or granules can be coated with materials that change their biological interactions, by altering surface charge, hydrophilicity and/or receptor-binding moieties. Such examples include, but are not limited to, chemical plasmas, peptides or carbohydrates, extracellular matrix components such as fibronectin or vitronectin or fragments thereof, poly-L-ornithine, polylysine and/or polyallylamines.

The osteoconductive pellets and/or granules may be added to a carrier, such as a pharmaceutically acceptable carrier. For example, the osteoconductive pellets and/or granules may be added to a carrier to form a paste or a putty. The active agent may be added to the carrier only, or to the osteoconductive pellets and/or granules and the carrier.

In one embodiment, the carrier is an aqueous carrier, such as water. The carrier may be an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, Krebs buffer, Dulbecco's PBS, or normal PBS; simulated body fluids, plasma platelet concentrate or tissue culture medium.

In addition to the carrier, the osteoconductive pellets and/or granules may be additionally or alternatively mixed with tissue scaffold material, such as the polymer particle-based scaffolds described in WO2017163072A1, which is herein incorporated by reference. For example, the osteoconductive pellets and/or granules of the invention may be mixed with and set into a solid scaffold material or a hydrogel. The osteoconductive pellets and/or granules of the invention may be mixed with polymer pellets (e.g. PLGA-based pellets/particles), which may be hollow polymer pellets, that may be suitable for use in bone repair.

According to a further aspect of the invention there is provided osteoconductive pellets and/or osteoconductive granules formed by the methods of the invention herein.

According to a further aspect of the invention there is provided an osteoconductive pellet, wherein the osteoconductive pellet is microporous and comprises or consists of at least 60% w/w calcium phosphate material, and optionally wherein the osteoconductive pellet comprises one or more concave surfaces, such as a hollow extending therethrough. The osteoconductive pellet may be in the form as described herein.

The osteoconductive pellet may comprise at least 70% w/w calcium phosphate material. The osteoconductive pellet may comprise at least 72% w/w calcium phosphate material. The osteoconductive pellet may comprise at least 80% w/W calcium phosphate material. The osteoconductive pellet may comprise at least 90% w/w calcium phosphate material. In another embodiment, the osteoconductive pellet comprise at least 95% w/w calcium phosphate material. In another embodiment, the osteoconductive pellet comprise at least 98% w/W calcium phosphate material. In another embodiment, the osteoconductive pellet comprise at least 99% w/w calcium phosphate material. In another embodiment, the osteoconductive pellet consists of 100% w/w calcium phosphate material.

The osteoconductive pellets may further comprise calcium deficient hydroxyapatite and/or octacalcium phosphate, for example on the surface of the osteoconductive pellets.

According to a further aspect of the invention there is provided an intermediate pellet suitable for the production of the osteoconductive pellet of the invention, wherein the intermediate pellet comprises or consists of less than 28% w/w polymer particles encapsulated within at least 60% w/w powdered calcium phosphate material.

In one embodiment, the intermediate pellet comprises or consists of less than 25% w/w polymer particles encapsulated within at least 70% w/w powdered calcium phosphate material. In another embodiment, the intermediate pellet comprises or consists of less than 25% w/w polymer particles encapsulated within at least 72% w/w powdered calcium phosphate material. In another embodiment, the intermediate pellet comprises or consists of less than 25% w/w polymer particles encapsulated within at least 75% w/w powdered calcium phosphate material. In another embodiment, the intermediate pellet comprises or consists of less than 22% w/w polymer particles encapsulated within at least 78% w/w powdered calcium phosphate material. In another embodiment, the intermediate pellet comprises or consists of less than 20% w/w polymer particles encapsulated within at least 80% w/w powdered calcium phosphate material. The powdered calcium phosphate material may not exceed 85%, 90% or 95% w/w of the intermediate pellet.

The polymer particles may be as described herein, such as ethylene vinyl acetate particles, or any other polymer particles that are capable of being burnt off in the sintering step (a) process.

According to a further aspect of the invention there is provided an osteoconductive granule, wherein the osteoconductive granule comprises or consists of β-TCP, and further comprises a surface topography comprising needle structures projecting from the surface of the granule.

The osteoconductive granules may further comprise calcium deficient hydroxyapatite and/or octacalcium phosphate, for example on the surface of the β-TCP granule.

The osteoconductive granules may be microporous. The osteoconductive granule may be in the form as described herein.

According to a further aspect of the invention there is provided an osteoconductive material, wherein the osteoconductive material comprises or consists of a plurality of osteoconductive pellets and/or granules according to the invention.

In one embodiment, the pellets may be non-uniformly orientated relative to each other. For example, the pellets may be randomly orientated. The hollow pellets may not be aligned relative to each other, for example in stacks to form honeycomb-like structures.

According to a further aspect of the invention there is provided a composition comprising the osteoconductive pellets and/or granules according to the invention.

The composition may comprise a carrier, for example as described herein.

According to a further aspect of the invention there is provided a method of treatment for bone repair or replacement, the method comprising the implantation of the osteoconductive pellets and/or granules according to the invention, or the composition according to the invention, into a subject in need thereof.

The implantation may be at a site for bone repair or replacement, such as a bone injury or defect.

According to a further aspect of the invention there is provided the osteoconductive pellets and/or granules according to the invention, or the composition according to the invention, for use as a medicament.

According to a further aspect of the invention there is provided the osteoconductive pellets and/or granules according to the invention, or the composition according to the invention, for use in bone repair or replacement in a subject.

The osteoconductive pellets and/or granules according to the invention may be for use in a method of treatment or prevention of bone-related disorders, such as a condition selected from osteoarthritis, spinal disk atrophy, bone cavities requiring filling, bone fractures requiring regeneration, bone cancer, or bone repair.

In other embodiments, the treatment is the repair of non-union fractures. In other embodiments, the treatment is spinal fusion. Spinal fusion is used to surgically treat vertebral abnormalities such as spinal curvatures (scoliosis or kyphosis), slipped discs (following discectomy), or fractures.

The osteoconductive pellets and/or granules according to the invention, or the composition according to the invention may be used to treat damaged bone tissue. In particular, to encourage or allow cells to re-grow in a damaged bone tissue. The invention may therefore be used in the treatment of bone tissue damage, including in the regeneration or reconstruction of damaged bone tissue.

### Definitions

The term “grain” used herein is a term of the art, and is understood to be a structure present on the surface of a pellet or granule, which can promote osteoconductivity. The grain may be in the form of a lump or projection from the surface, or a granule bonded to the surface. It is understood that grain sizes of <1.5 μm are advantageous for ectopic bone growth.

“Osteoinstructive” refers to the property of a material with biochemical and physical cues which modulates osteo cell (osteoprogenitor/osteoblast) behaviour.

These cells are sensitive to biological/chemical/physical signals within their immediate microenvironment. These signals instruct cells adhesion/migration/intracellular activity and signalling pathways/gene expression (see Gonzalez-Fernandez T, Sikorski P, Leach J K. Bio-instructive materials for musculoskeletal regeneration. Acta Biomater. 2019 Sep. 15; 96:20-34. doi: 10.1016/j.actbio.2019.07.014. Epub 2019 Jul. 11. PMID: 31302298; PMCID: PMC6717669, which is herein incorporated by reference). This differs from “osteoinductive” where a bioactive molecule will trigger bone formation.

“Osteoinduction” is the process by which osteogenesis is induced. The methods herein may be for producing osteoinductive pellets and/or granules, for example when a bioactive or growth factor is incorporated.

“Osteoconductivity” in relation to a material, such as the pellets and granules described herein, is the ability for bone-forming cells to move across the material and replace the material with bone over time.

The term “burn off” or similarly “burnt off” is intended to refer to the removal of a substance, such as removal of the polymer, by heat, for example by incineration. The substance to be burnt off may be removed by one or more of incineration, melting and evaporation.

“Sintering” is the process of compacting and forming a solid mass of material by heat or pressure without melting it to the point of liquefaction.

“Hydrothermal treatment (HTT)” is the process of applying high temperatures and/or pressures in a high humidity environment. Autoclaving is an example of a hydrothermal treatment, and such a term may be used interchangeably with HTT.

“Wicking” means acting to absorb or draw off liquid by capillary action.

Macropores may be considered to be greater than 100 μm. Mesopores may be considered to be any pore size between Macro and Micro. Micropores may be considered to be less than 20 μm, or more preferably less than 10 μm. Nanopores may be considered to be less than 1 μm

Interconnected pores may be considered to be pores that have fluid connectivity to other pores. The skilled person will recognise that the pores may have necks/throats, for example where a pore is connected to another pore by a narrower channel. Interconnected pores are considered to be capable of wicking a fluid.

The term “room temperature” is intended to refer to a temperature of from about 15° C. to about 25° C., such as from about 20° C. to about 25° C.

The skilled person will appreciate that preferred features of any one embodiment and/or aspect of the invention may be applied to all other embodiments and/or aspects of the invention.

### EXPERIMENTAL DATA

### Example 1-Development of the Certoss™ Conduit Product

### Key Conclusions

Hollow pellets comprising of 80% (w/w) β-TCP and 20% (w/w) ethylene vinyl acetate (purchased as ELVAX™ polymer from Dow) can be prepared using a hot melt extrusion technique.

A small ‘pre-feedstock’ which is extruded (and discarded) before the main feedstock ensures that there are no blockages in the die which prevents pressure build-up and enables the main feedstock to successfully extrude.

The resulting hollow pellets can be converted into Certoss™ Conduit pellets by a two-stage sintering process.

The particle size of the β-TCP influences the quality of the Certoss™ Conduit pellet. Optimum mean particle size is about 2 to 10 μm. Smaller particle sizes may also work.

Sintering temperature affects the surface microporosity and strength of the Certoss™ Conduit pellet. The optimum temperature and time is 1050° C. for 6-10 hours

Certoss™ Conduit pellets can be hydrothermally treated to cause the formation of needles on the surface.

The presence of α-TCP is essential during the HTT process but, the performance varies between different suppliers.

Certoss™ Conduit pellets can adsorb protein to their surfaces.

### Aim

Developing an osteoinductive ceramic material that has both macroporosity (achieved by open lumens within β-TCP pellets) and interconnected microporosity within each pellet (achieved by the optimised sintering of β-TCP powder). The particle size of the β-TCP powder can also dictate the surface topography of the pellets and can influence the manufacturability of the product.

The aim is to describe the early development of this material to the point of being suitable for inclusion in an ectopic pre-clinical study. The product has the working name of Certoss™ Conduit.

### SUMMARY

Certoss™ Conduit is comprised either of β-TCP powder which is purchased ‘off the shelf’ having a particle size of <25 micron or of β-TCP powder that has been milled in-house down to <4 micron. The β-TCP powder is dry mixed with ELVAX™ ethylene vinyl acetate (milled to <1 mm) to create a feedstock for hot melt extrusion (HME). Hollow pellets having an aspect ratio of 1.4 to 1.9 are created by HME, they are then sintered in a furnace in a two-step process. This process slightly reduces the aspect ratio.

The sintered pellets may then be subjected to a hydrothermal treatment (HTT) method which encourages the growth of surface needles. This surface treatment is reported to initiate osteoinductivity of the ceramic in-vivo. However, the Certoss™ Conduit manufactured using the finest grain powder (around 2 micron) may not need HTT as literature reports that a ceramic having a sub-micron grain/pore size may also be osteoinductive.

Further information regarding the methodologies described is set out below (see Reference LOCSOP124.01 and Reference LOCSOP125.01).

### Conclusions

Certoss™ Conduit is manufactured from β-TCP powder which is mixed with ELVAX™ polymer and hot melt extruded to produce a hollow tube which is cut up into small pellets. The pellets then undergo a two-stage sintering process to remove the ELVAX™ polymer and sinter the powder grains together leaving a hollow cylindrical pellet which has interconnected microporosity.

β-TCP content influences the quality of the Certoss™ Conduit pellet. At 70% (w/w) β-TCP the resulting pellets will not hold their shape during the sintering process. At 80% (w/w) β-TCP the pellets sinter to produce a hollow pellet. This is apparent when a 7 micron β-TCP powder is used.

The high β-TCP powder content of the feedstock can cause blockages within the die resulting in an off-centre lumen. If a small amount of ‘pre-feedstock’ is used having a higher ELVAX™ content, the blockage issues are avoided if the particle size are no less than 3 micron (average size).

β-TCP particle size also influences the HME process. Below 3 micron (average size) the extruder blocks, even if a pre-feedstock is used, and no material can be produced. Below 4 micron (average size) the torque increases but, extrusion is possible and the resulting pellets are of excellent quality and maintain their shape during the sintering process. Most of the work to optimize the methods was performed using 7 micron β-TCP (Certificate of Analysis (CoA)) pellets made from this β-TCP sintered but stuck together and the shape was slightly altered. Using β-TCP with a particle size of 15 micron (CoA) resulted in weak pellets that fall apart upon handling.

The parameters for the HME can be set to ensure that ELVAX/β-TCP pellets made can have an aspect ratio (AR) of between 1.4 and 1.9. The AR is lowered once the pellets have undergone the sintering process.

The sintering process affects the surface morphology of the resulting Certoss™ Conduit pellet. The optimum sintering temperature is 1050° C. for 6-10 hours.

All the Certoss™ Conduit pellets regardless of β-TCP size and sintering time can wick fluid which demonstrates interconnected microporosity.

α-TCP is an essential component during the hydrothermal treatment but, the material supplied by Himed and is milled in-house shows superiority over the material supplied by Plasma Biotal at a defined size (20.3 μm).

All the pellets with adsorb protein to their surface when incubated at 37° C. in the presence of culture media containing fetal bovine serum.

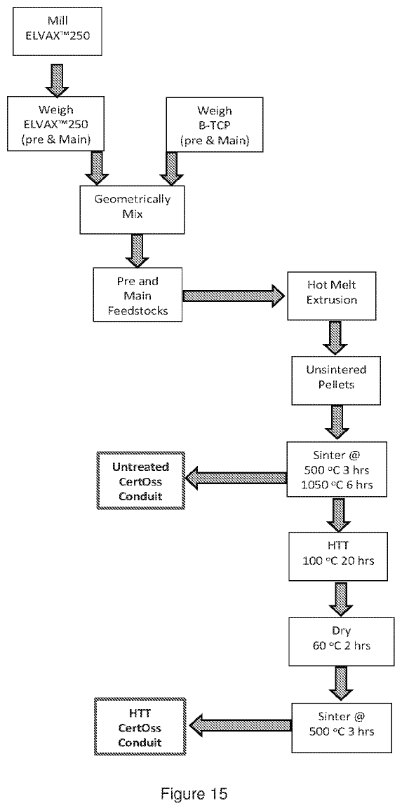

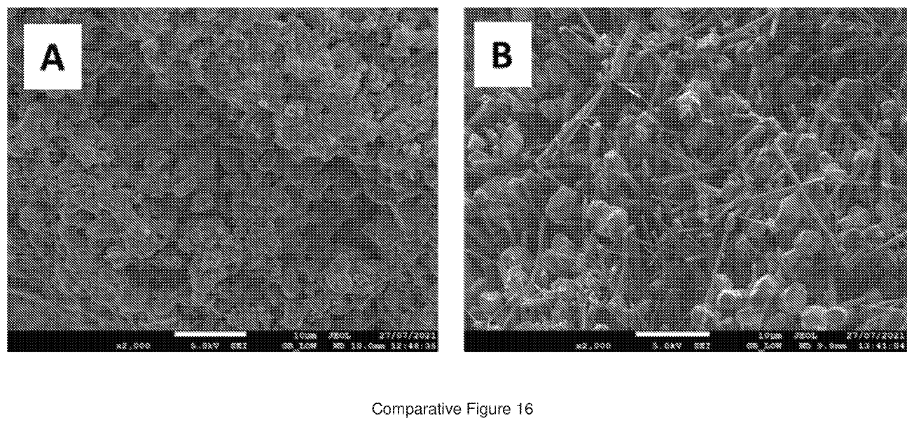

A summary of the manufacturing process is shown in FIG. 15.

### Method and Materials

See list of materials used in Table 1.

TABLE 1
Materials used in the development of Certoss ™ Conduit.

| Reagent | Supplier | Cat/LOT No. |
|---|---|---|
| β-TCP | CaP Biomaterials | ‘7 micron batch’ TCPSV032020A |
| β-TCP | CaP Biomaterials | ‘15 micron batch’ TCPSV110520A |
| ELVAX ™250 | Dow Corning | |
| α-TCP | Himed | 220113G 500-1000 micron. |
| | | Milled/sieved to <150 micron |
| α-TCP | Plasma | P3995 SD S1000 HT |
| | Biotal | LOT7 20.3 micron |

### Methods

The main parameters identified for investigation were:
The settings for the HME process
The concentration of β-TCP within the HME feedstock.
The particle size of β-TCP within the HME feedstock.
The sintering of the pellets post-HME.
The HTT of the pellets post sintering.

All of these parameters are inter-linked but best efforts have been made to optimise each one using minimal iterative steps.

### Optimisation of HME Settings

The extruder settings were optimised during each run based on the material being extruded. These settings were then used as a starting point for the next run. As more batches were made fewer changes were need during the processing which allowed the generation of a final settings profile as follows:

Hopper feed set initially at 4% for the ‘pre-feedstock’ then reduced to 1.6% for the main feedstock.

Temperatures set at 90, 130, 130 130° C.

Screw speed set initially at 20 rpm for pre-feedstock then reduced to 15 rpm for the main feedstock.

Conveyor set at minimum speed.

Pelletiser set at 0.4 m/min pull and 3 mm cut.

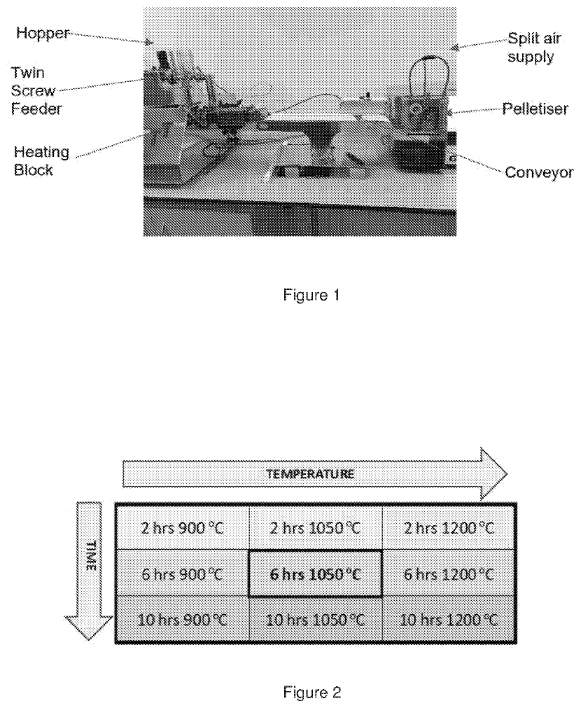

The pelletiser set as close as possible to the conveyor and the conveyor set as close as possible to the die. See FIG. 1 for final set up.

### Optimisation of β-TCP Content

The initial trial using β-TCP (‘7 micron’) and ELVAX™ used a feedstock comprising of a 50:50 mix of each by weight. The subsequent trials with the same material were performed using a 70:30 mix and an 80:20 mix by weight. These mixes are also referred to as 50%, 70% and 80% (w/w) β-TCP.

The ELVAX material was milled using the Fritsch Pulverisette 11 (see Reference LOCSOP125.01, ‘Milling ELVAX™ 250’) and sieved to below 1 mm. The ELVAX™ was geometrically mixed with the β-TCP to generate the feedstock.

Once the HME was set up (see Reference LOCSOP125.01, ‘Feedstock Manufacture’ and ‘Hot Melt Extrusion’) the feedstock was added to the HME screws. For the first few trials, this was performed manually but in later trials, the automatic twin screw feeder was used.

Eight batches of Certoss™ Conduit were produced (not including the initial 50:50) trial. The batches were designated ‘HOLxxx’ to signify ‘HOLlow’ pellets. The HME settings for the first six batches are shown in Table 2. Batches HOL 004, 005 and 006 are made using the same settings with differing amounts of feedstock to create enough hollow pellets for further work.

The advantage of a ‘pre-feedstock’ as well as a ‘main-feedstock’ will become evident in the results section.

TABLE 2
Material and HME settings for batches HOL 001-HOL 006

| Sample No. | Material | Final HME settings |
|---|---|---|
| HOL 001 | 70% β-TCP powder (7 | 80 120 120 120 °C |
| | um) | 10 rpm |
| | 30% ELVAX ™ 250 (<1 | Pull 0.5 mm/min cut 1.2 mm |
| | mm) | Pelletiser distance 20 cm |
| HOL 002 | 80% β-TCP powder (7 | 80 120 120 120 °C |
| | um) | 10 rpm |
| | 20% ELVAX ™ 250 (<1 | Pull 0.5 mm/min cut 1.5 mm |
| | mm) | Pelletiser distance 20 cm |
| HOL 003 | 80% β-TCP powder (7 | 80 120 120 120 °C |
| | um) | 10 rpm |
| | 20% ELVAX ™ 250 (<1 | Pull 0.5 mm/min cut 1.5 mm |
| | mm) | Pelletiser distance 20 cm |
| HOL 004 | 80% β-TCP powder | 90 130 130 130 °C |
| | (7 um) | 15 rpm |
| | 20% ELVAX ™ 250 (<1 | pull 0.18 m/min cut 4.60 mm |
| | mm) | Pelletiser as close as possible |
| | | (see FIG. 1) |
| HOL 005 | 50% b-TCP (7um) | 90 130 130 130 °C |
| (now | 50% ELVAX ™ (<1 mm) | 15 rpm |
| includes | Followed by | pull 0.18 m/min cut 4.60 mm |
| small | 80% β-TCP powder | Pelletiser as close as possible |
| amount of | (7 um) | |
| pre- | 20% ELVAX ™ 250 (<1 | (see FIG. 1) |
| feedstock) | mm) | |
| HOL 006 | 50% β-TCP (7 um) | 90 130 130 130 °C |
| (now | 50% ELVAX ™ (<1 mm) | 15 rpm |
| includes | Followed by | pull 0.18 m/min cut 4.60 mm |
| small | 80% β-TCP powder | Pelletiser as close as possible |
| amount of | (7 um) | (see FIG. 1) |
| β-TCP | 20% ELVAX ™ 250 (<1 | |
| powder | mm) | |
| pre- | | |
| feedstock) | | |

### Optimisation of Sintering Method: Using the ‘7 Micron’ β-TCP Powder

The batch HOL 006 was used throughout the sintering study to compare differing sintering times and temperatures on the same material. A sintering temperature of 1050° C. for 6 hours was selected early in Certoss™ Conduit development. However, prior to this, the material was held for 3 hours at 500° C. to burn off the EVAX™ polymer leaving behind only the β-TCP.

FIG. 2 shows the different sintering times and temperatures trialled. Each sample was imaged by SEM.

Optimisation of 8-TCP particle size (<2, <4, 7 and 15 micron) Two batches of β-TCP (<25 μm) particles were available, the CoAs stated the particle sizes were ‘7-micron’ and ‘15 micron’. A sample of each of these was milled using the Fritsch P6 planetary ball mill (see Reference LOCSOP126). The sizes achieved are in Table 3 and will be described as <3 and <4 micron.

TABLE 3
Sizing of milled β-TCP powders

| Name | mean | D10 | D50 | D90 |
|---|---|---|---|---|
| <3 | 1.66 | 0.69 | 1.49 | 2.86 |
| micron | | | | |
| <4 | 2.18 | 0.87 | 1.96 | 3.95 |
| micron | | | | |

The manufacturing parameters for the 4 batches trialled are shown in Table 4.

TABLE 4
Material and HME settings for batches HOL 006-HOL 008

| Sample No. | Material | Final HME settings |
|---|---|---|
| HOL 006 | 50% β-TCP (7 um) | 90 130 130 130 |
| | 50% ELVAX ™ (<1 mm) | 15 rpm |
| | Followed by | pull 0.18m/min cut |
| | 80% β-TCP powder (7 um) | 4.60 mm |
| | 20% ELVAX ™ 250 (<1 mm) | Pelletiser as close as |
| | | possible |
| | | (see FIG. 1) |
| HOL 007 | 50% β-TCP (15 um) | 90 130 130 130 |
| | 50% ELVAX ™ (<1 mm) | 15 rpm |
| | Followed by | pull 0.39 m/min cut |
| | 80% β-TCP powder (7 um) | 3.00 mm |
| | 20% ELVAX ™ 250 (<1 mm) | Pelletiser as close as |
| | | possible |
| | | (see FIG. 1) |
| HOL 008 | 50% β-TCP (<4 um) | 90 130 130 130 |
| | 50% ELVAX ™ (<1 mm) | 15 rpm |
| | Followed by | pull 0.40 m/min cut |
| | 80% β-TCP powder (7 um) | 3.00 mm |
| | 20% ELVAX ™ 250 (<1 mm) | Pelletiser as close as |
| | | possible |
| | | (see FIG. 1) |
| NOT | 50% β-TCP (<3 um) | n/a |
| ASIGNED- | 50% ELVAX ™ (<1 mm) | |
| FAILED | Followed by | |
| RUN | 80% β-TCP powder (7 um) | |
| | 20% ELVAX ™ 250 (<1 mm) | |

### Hydrothermal Treatment to Generate Surface Needles

The method to hydrothermally treat sintered Certoss™ Conduit is described below (see Reference LOCSOP125.01, ‘Hydrothermal Treatment’). The method employs the use of Baoshishan hydrothermal reactor vessels.

The only difference in the method for Certoss™ Conduit (see Reference LOCSOP125.01, ‘Hydrothermal Treatment’) is that half the amount of α-TCP is used as halved because there are fewer Certoss™ Conduit pellets in the reactor vessels that Certoss™ Granules (see Reference LOCSOP124.01).

### Characterisation Methods

The size of the pellets were measured before and after the sintering step to give a measurement for aspect ratio.

The pellets were imaged by SEM and light microscopy.

The pellets were tested for interconnected microporosity by allowing coloured liquid to wick through the pellet by capillary action.

Protein adsorption to the pellets was measured by 100 mg of each ceramic sample being weighed directly into a well of a 24 well tissue culture plate. 2 ml of complete tissue culture media (DMEM+10% FBS+PS) being added to each well including control wells with no sample. The plate was then incubated for 24 hrs at 37° C./5% CO₂. All the samples were then thoroughly washed 3× with 2 ml of DPBS. A 1% v/v solution of Triton-X was added to the wells (0.5 ml) and the plate was left for 30 minutes at room temperature on a plate shaker to gently agitate.

An assessment of the protein concentration in each supernatant was determined by following the manufacturer's instructions for the ThermoFisher Micro BCA kit.

A solution of 1% v/v Triton-X was used to dilute the calibration standards.

### Results

### Optimisation of β-TCP Content

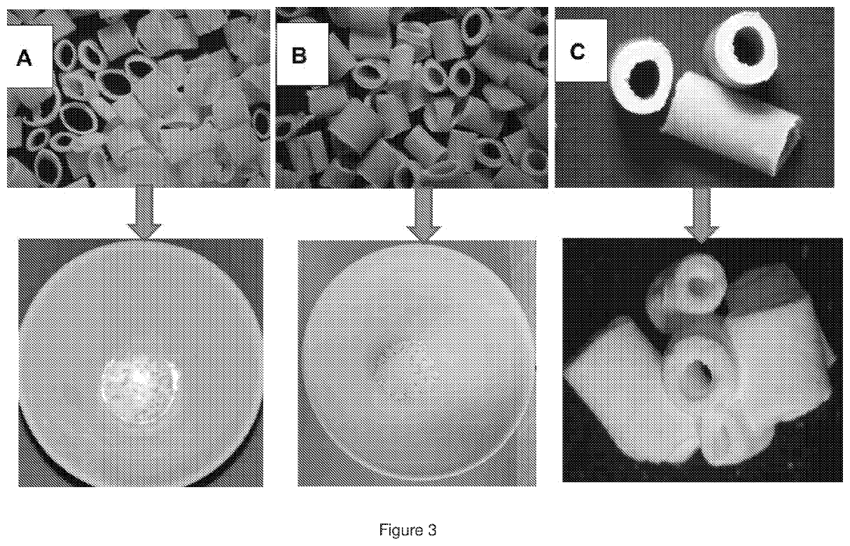

The initial trial using 50:50 β-TCP/ELVAX™ produced hollow pellets with very thin walls. The pellets were brittle and did not withstand the sintering process (FIG. 3A). The trial HOL 001 using a feedstock comprising of 70% (w/w) β-TCP yielded pellets of a similar diameter to the initial trial with slightly thicker walls but of greater length having a mean aspect ratio of 1.42. However, this material also did not survive the sintering process FIG. 3B).

Once the HME parameters were further optimised (see ‘Optimisation of HME settings’ above), including the use of a wider cone at the die to enable a thicker pellet wall, larger pellets were generated that did survive the sintering process although, many of the pellets sintered together but, these could be separated moderately easily.

Once pellets comprising of 80% (w/w) β-TCP have been generated, the β-TCP content was not increased further.

It is currently unknown whether 90% (w/w) β-TCP could be achieved with this 7 micron particle size. But, it is likely to block the die due to not enough ELVAX™ being available.

A summary of the sizing data is shown in Table 5. The sizing of the first initial batch was not recorded. Length and diameter were measured using calipers.

TABLE 5
Size analysis of HOL 001 and HOL 005

| Batch | Length (mm) | Diameter (mm) | Aspect ratio |
|---|---|---|---|
| HOL 001 | 1.42 ± 0.12 | 1.02 ± 0.17 | 1.42 ± 0.25 |
| HOL 005 | 3.68 ± 0.24 | 2.29 ± 0.11 | 1.65 ± 0.12 |

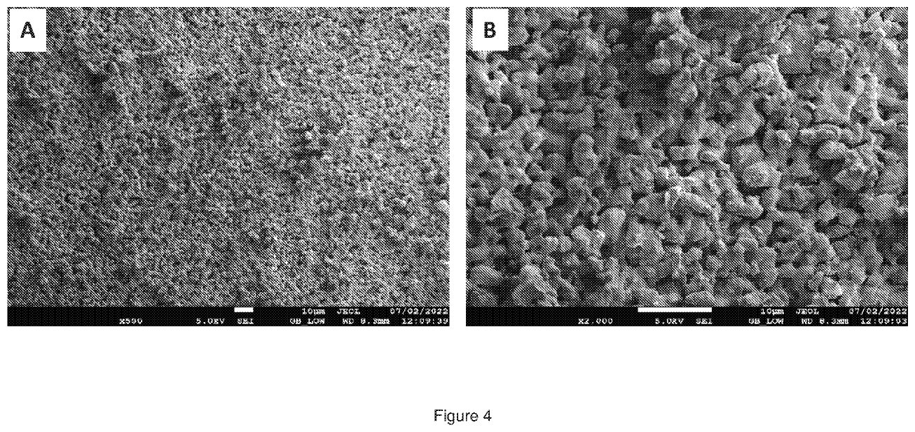

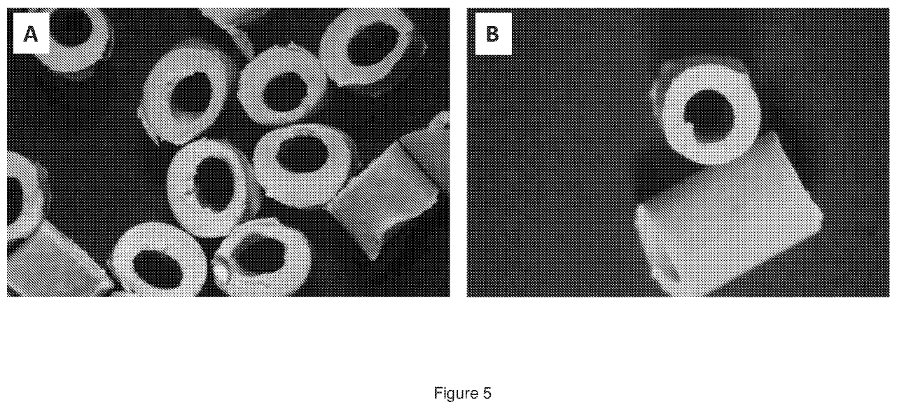

SEM imaging was performed to investigate the surface topography of the sintered material. See FIG. 4 showing the surface at ×500 and ×2000 magnification.

The ELVAX™ has burnt off leaving pores and the β-TCP powder has sintered together creating a microporous surface.

### Inclusion of a ‘Pre-Feedstock During HME Process

Although batches HOL02-04 extruded, it was noted that torque, which is a measurement of pressure in the die was higher than usually seen and the Conduit pellets tended to have an ‘off-centre’ lumen despite the pin in the die being seated correctly. Upon cleaning the die, a few of the holes (that equalize the pressure around the pin) were blocked with compacted powder.

A small (5 g) pre-feedstock solved this problem by extruding first and ensure that the holes were not blocked for the main feedstock to follow. FIG. 5 shows the difference in pellet shape between HOL 002 (without a pre-feedstock) and HOL 005 (with a pre-feedstock).

### Optimisation of Sintering Method: Using the ‘7 Micron’ β-TCP Powder

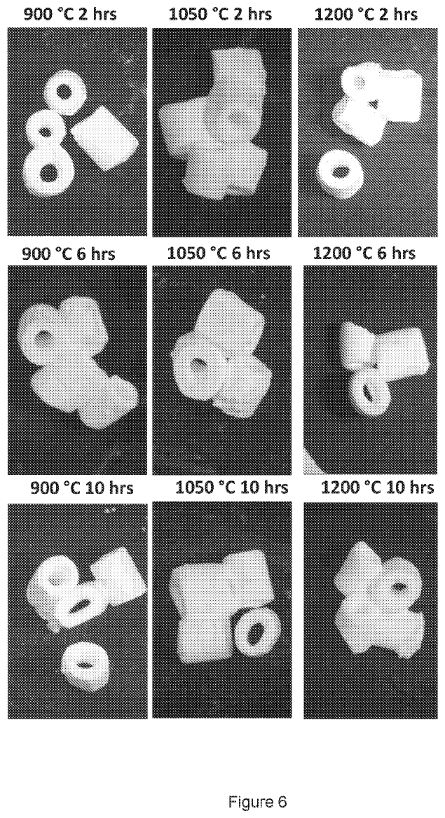

All of the conditions resulted in sintered pellets that in most cases held their shape but also sintered together (See FIG. 6). However, upon handling it was evident that they had different strengths. A quick test to assess the strength was to crush a pellet from each group and score it subjectively on a scale of 1 to 5 where l is extremely fragile and 5 is difficult to manually crush. The results of this are shown in Table 6.

TABLE 6
Subjective strength scores for Certoss ™ Conduit pellets

| Sample | Strength Score |
|---|---|
| HOL 6B 1200 °C 2 hrs | 5 |
| HOL 6C 1200 °C 6 hrs | 5 |
| HOL 6D 1200 °C 10 hrs | 5+ |
| HOL 6E 1050 °C 2 hrs | 3 |
| HOL 6F 1050 °C 6 hrs | 3 |
| HOL 6G 1050 °C 10 hrs | 4 |
| HOL 6H 900 °C 2 hrs | 2 |
| HOL 6I 900 °C 6 hrs | 2 |
| HOL 6J 900 °C 10 hrs | 2 |

All of the samples were assessed for interconnected microporosity by the wicking test. All pellets wicked liquid throughout their structure. HOL6D was slightly slower to wick than the rest. See the ‘Characterisation Methods’ section and the associated results for details.

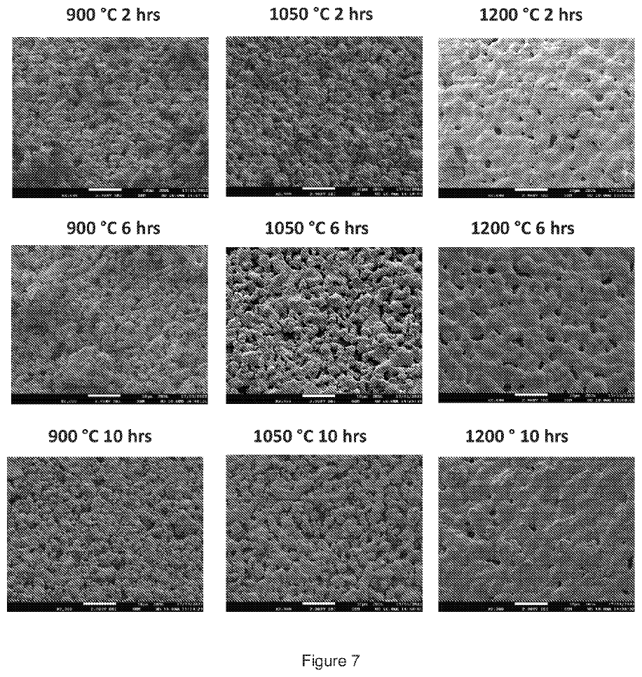

FIG. 7 shows the surface topography via SEM for all of the sintered pellets at ×2000. Sintering temperature appears to have more effect on the surface than sintering time. At 1200° C., the particles are obviously more tightly sintered together and this is reflected in the strength scores.

A sintering temperature of 1050° C. appears to be optimum as at 900° C. the Certoss™ Conduit pellets are very weak and easily crumble back to a powder.

There is a potential to increase the sintering time up to 10 hours at 1050° C. if a slightly stronger pellet is required.

### Optimisation of β-TCP Particle Size (<2, <4, 7 and 15 Micron)

All the optimisation work was performed on the ‘7 micron batch’ (TCPSV032020A).

A 15 micron batch (TCPSV110520A) was also available and used to generate batch HOL 007.

The two milled samples (<4 micron and <3 micron) were used to generate two further batches.

HOL 006 was the batch having the current lead parameters and was prepared as a repeat of HOL 005 to determine whether the process could be scaled up. Approximately 63 g of HOL 006 were successfully prepared from a 75 g feedstock (80% yield).

The torque value on the HME is an indication of the pressure at the die and for batches HOL 006 and HOL 007, the torque did not exceed 3 Nm throughout the extrusion process.

It was noted that for batch HOL 008 where the <4 micron milled β-TCP was used, the torque rose to 6.5 Nm during the processing.

When the finest milled β-TCP (<3 micron) was used the torque exceeded the maximum allowed (14 Nm) and the process was automatically halted. The holes in the die were completely blocked and no material could be collected. The fine material had compacted in all of the holes. This was probably due to there not being enough ELVAX™ to coat the higher surface area of the small particles.

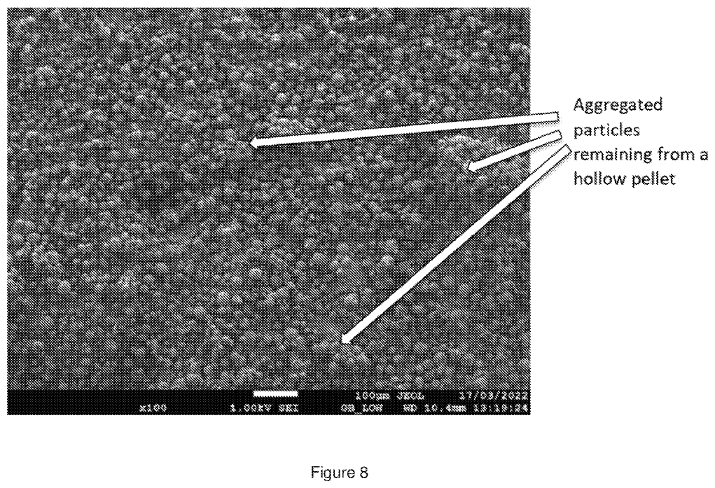

The batch HOL 007 (manufactured with ‘15 micron’ material) did not sinter sufficiently for the hollow pellets to hold their shape. They easily returned to a powder upon handling. The SEM below in FIG. 8 shows a small number of aggregated particles that remain.

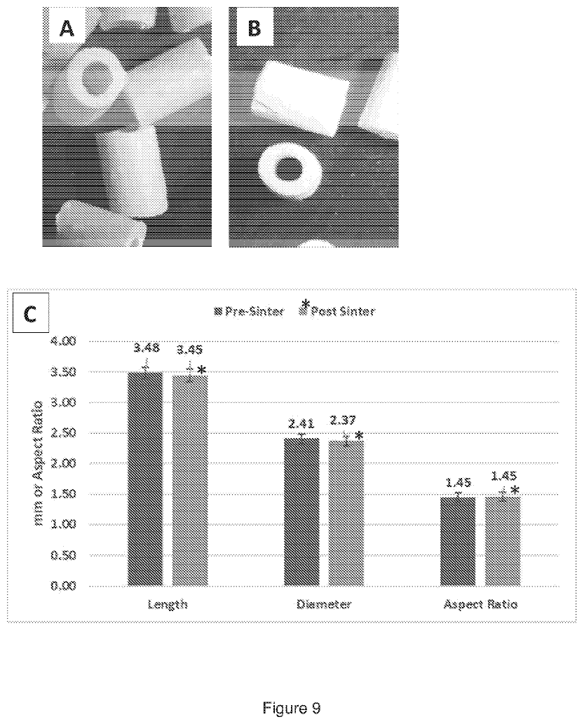

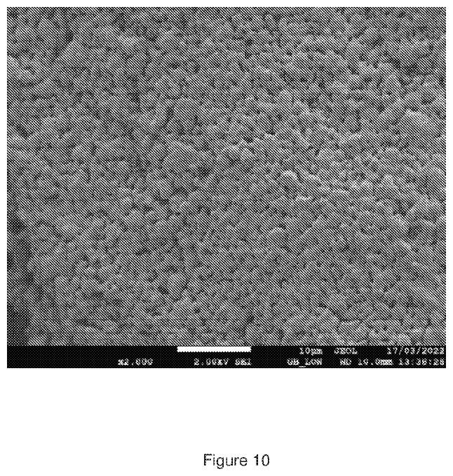

The batch HOL 008 comprising of the milled (<4 micron) β-TCP generated pellets which when sintered at 1050° C. for 6 hours did not stick together, did not change their shape and retained the same aspect ratio (See FIG. 9).

The surface of these pellets demonstrated microporosity (See FIG. 10) and a wicking test showed that the pores were interconnected.

### Hydrothermal Treatment to Generate Surface Needles

Sintered Certoss™ conduit pellets were subjected to a hydrothermal treatment (see Reference LOCSOP125.01 ‘Hydrothermal Treatment’).

Both α-TCP supplied by HiMed and milled in house and α-TCP supplied by Plasma Biotal were used in the reactors. Half the amount of α-TCP generally used for Certoss™ Granules (see Reference LOCSOP124.01) was used for Certoss™ Conduit (ss Reference LOCSOP125.01) because far fewer pellets that granules by mass were inside the reactor.

1 g Certoss™ Conduit pellets+100 mg α-TCP+50 ml of water was the amounts used.

The treatments were successful, but needle coverage was often patchy. The introduction of a step to swirl the reactors to re-settle the pellets appeared to improve the needle coverage.

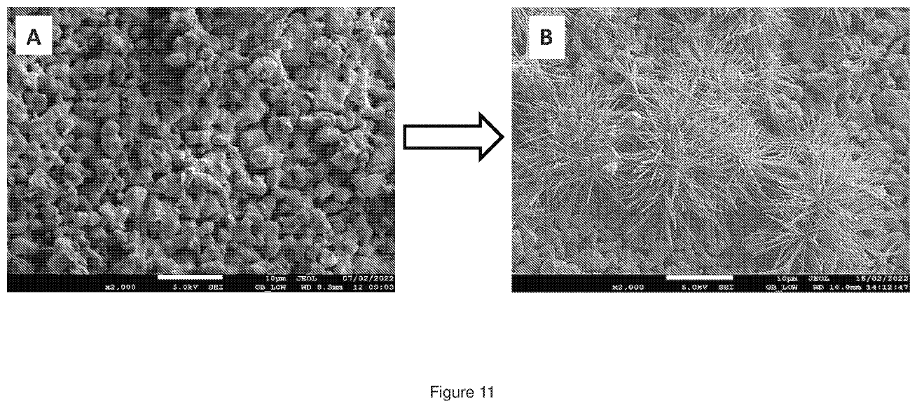

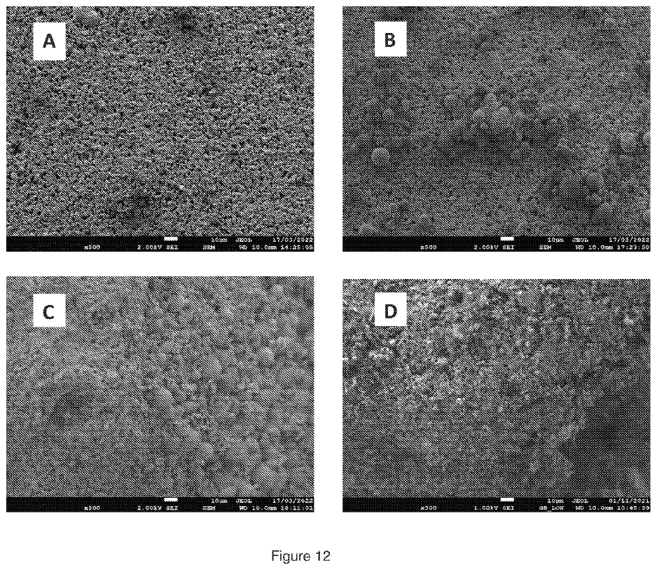

FIG. 11 shows the surface of a Certoss™ Conduit pellet pre- and post HTT (Batch HOL 005B and HOL 005E).

It was noted as part of the sintering study that when α-TCP supplied by Plasma Biotal was used (20.3 micron) no needles formed on the surface and this was true for Certoss™ Granules that were imaged at the same time. FIG. 12 shows the untreated Certoss™ Conduit surface, the HTT Certoss™ Conduit surface and the HTT Certoss™ Granules surface none of with display needle formation. It also shows the Certoss™ Granule surface after HTT with Himed α-TCP (milled to <50 micron) which do show needle formation. The spherical structures on the surface are likely to be the α-TCP particles. It is noted that the α-TCP supplied by Plasma Biotal may not be soluble, which may result in no needle formation.

### Characterisation Methods

### Size Analysis

Once the HME parameters had been optimized, the resulting pellets had aspect ratios ranging from 1.5 to 1.9 but, when the pellets were sintered, the aspect ratio altered mainly due to the pellets changing shape (see FIG. 6) and having a wider diameter. The size analysis for batches HOL 004, HOL 005 and HOL 006 is shown in Table 7. Length and diameter were measured using calipers. Only batch HOL 008 manufactured with β-TCP having a particle size of <4 micron did not change shape during the sintering process (see FIG. 9). Lumen and wall size analysis is shown in Table 7a. Lumen diameter and wall thickness were measured using calipers. From all of the measured pellets, the maximum value and minimum value are reported. The average is the mean value.

TABLE 7
Size analysis for three batches of Certoss ™ Conduit pellets

| Batch | Length (mm) | Diameter (mm) | Aspect ratio |
|---|---|---|---|
| HOL 004 | 3.59 ± 0.15 | 1.95 ± 0.16 | 1.85 ± 0.14 |
| Pre-Sinter | | | |
| HOL 004 | 3.16 ± 0.21 | 2.06 ± 0.23 | 1.55 ± 0.16 |
| Post sinter | | | |
| HOL 005 | 3.68 ± 0.24 | 2.29 ± 0.11 | 1.65 ± 0.12 |
| Pre-sinter | | | |
| HOL 005 | 3.44 ± 0.33 | 2.49 ± 0.18 | 1.39 ± 0.15 |
| Post-Sinter | | | |
| HOL 006 | 3.04 ± 0.31 | 2.00 ± 0.24 | 1.53 ± 0.20 |
| Pre-sinter | | | |
| HOL 006 | 2.61 ± 0.22 | 2.19 ± 0.28 | 1.20 ± 0.16 |
| Post-Sinter | | | |

### Lumen and Wall Size Analysis

TABLE 7a
Lumen and well size analysis

| | | SD: | | |
|---|---|---|---|---|
| | Average | standard | Max value | Min |
| | (mm) | deviation | (mm) | value (mm) |
| Lumen | 0.43 | 0.06 | 0.57 | 0.29 |
| diameter | | | | |
| Wall | 0.70 | 0.09 | 0.86 | 0.55 |
| thickness | | | | |

### Porosity

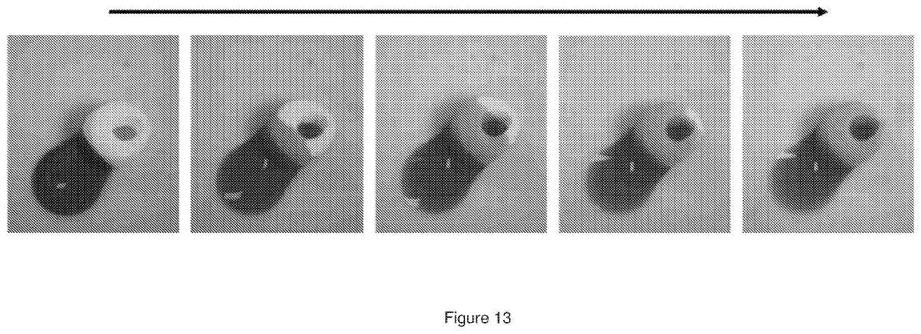

It was demonstrated that a small amount of food colouring could wick through a Certoss™ Conduit pellet indicating interconnected microporosity. The wicking time was less than 30 seconds (see FIG. 13).

### Protein Adsorption

Three different batches of Certoss™ Conduit pellets both prior and post HTT were incubated with culture media containing 10% FBS. The negative control was tissue culture plastic (TCP).

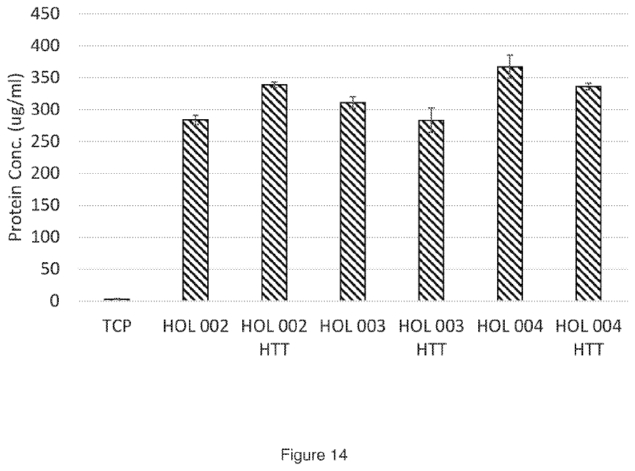

All samples showed good adsorption of protein, only batch HOL 002 showed an improvement after HTT (see FIG. 14).

### Example 2-Development of the Certoss™ Granules Product

### Key Conclusions

Base granules comprised of β-TCP can be subjected to a hydrothermal treatment which generated fine needles at the surface.

The presence of α-TCP in the reaction vessel improved the needle formation and coverage but the particle size and amount of α-TCP was of less importance.

Hydrothermal treatment of β-TCP granules to form surface needles could surprisingly be performed at 100° C.

Protein adsorbed readily to both untreated β-TCP granules and hydrothermally treated β-TCP granules when they were incubated at 37° C. in complete culture media containing 10% FBS for 24 hours. The protein adsorption was enhanced on the treated granules.

The pH within the reaction vessel affected the needle size with pH 11 causing the formation of the smallest needles (0.1×1.4 μm).

Cells adhered to hydrothermally treated β-TCP granules as well as untreated β-TCP granules and these can be visualized by SEM.

Metabolic activity of cells was slightly enhanced when granules having a needle size similar to MagnetOs™ were tested (0.4×8.8 μm). Needle size was measured by scanning electron microscopy (SEM) to image the surface of the pellet or granule, including the needle projections. Software such as Image-J software was then used to process and analyse the image produced by SEM.

### Aim

Developing an osteoinductive ceramic material by utilising a hydrothermal treatment method to alter the surface topography of commercially available porous granules.

The aim is to describe the early development of this material to the point of being suitable for inclusion in an ectopic pre-clinical study. The product has the working name of Certoss™ Granules.

### SUMMARY

Certoss™ Granules are comprised of porous β-TCP granules which are purchased ‘off the shelf’ having a particle size of 1-2 mm. These granules are subjected to a hydrothermal treatment (HTT) method in the presence of α-TCP which encourages the growth of surface needles. This surface treatment is reported to initiate osteoinductivity of the ceramic in-vivo.

The main parameters identified for investigation during Certoss™ Granule development were:
The base ceramic granule (BCP or β-TCP)
The time and temperature for optimum hydrothermal treatment
The presence/absence of α-TCP in the hydrothermal chamber
The amount and particle size of the α-TCP in the hydrothermal chamber

All of these parameters are inter-linked but best efforts have been made to optimise each one.

The characterisation methods for the test granules were:
SEM imaging
Protein adsorption
Cell attachment

### Conclusions

Although hydrothermal treatment to modify the surface of BCP granule comprised of 20%-HA/80%-β-TCP worked very well, granules comprised of β-TCP required alternative HTT conditions.

The presence of α-TCP within the reactor vessel greatly enhanced needle generation on the β-TCP granule surface. However, the particle size of the α-TCP and amount of α-TCP present was of less importance.

The longer the HTT, the larger the surface needles but when a temperature of 100° C. was used, needles of a more optimum size (similar to MagnetOs™) were generated.

Protein will adsorb readily to both untreated β-TCP granules and the amount slightly enhanced when HTT β-TCP are treated with complete culture media containing 10% FBS for 24 hours.

The pH within the reaction vessel affected the needle size with pH 11 causing the formation of the smallest needles (0.1×1.4 μm).

Cells adhered to hydrothermally treated granules as well as untreated granules and these could be visualized by SEM. Metabolic activity of cells was slightly enhance when granules having a needle size similar to MagnetOs™ were tested (0.4×8.8 μm).

### Method and Materials

TABLE 8
Materials used in the development of Certoss™ Granules.

| Reagent | Supplier | Cat/LOT No. |
|---|---|---|
| BCP 20/80 | CaP | 0.5-100 mm Lot |
| | Biomaterials | BPDP040721A |
| β-TCP | CaP | 1-2 mm Lot |
| | Biomaterials | TCPDP052120A |
| β-TCP | Himed | 1-2 mm Lot SWB220221 |
| α-TCP | Himed | 220113G 500-1000 μm. |
| | | Milled/sieved as required |

### Methods

### Baoshishan Hydrothermal Reactor Vessels

The hydrothermal treatments are performed in 100 ml Baoshishan hydrothermal reactors.

The test materials and water are placed within the white PTFE liner which is placed into the steel vessel and sealed via the screw thread. The whole vessel is placed in an oven at the required temperature for the set time. Once cooled, the vessel can be opened, the water is removed, and the granules dried at 60° C. to remove any remaining water. The granules are then carefully transferred to a crucible and heated in a furnace to 500° C. for 3 hours to strengthen the pellets.

The details for the test conditions are described in the results section.

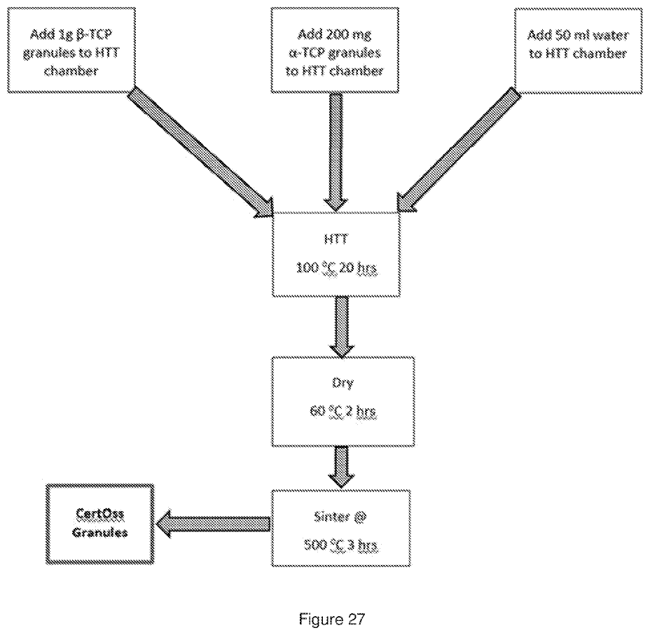

A schematic showing the manufacturing process is shown in FIG. 27.

### Results

### The Base Ceramic Granule (BCP or β-TCP)

The initial work on hydrothermal treatments were performed on a commercially available BCP granule (20% HA/80% β-TCP) as this is the most common ceramic used in the art. It is known in the art that the process does not work well on a pure β-TCP granule.

Comparative FIG. 16 shows the alteration in the surface topography after HTT of a BCP granule by SEM. Many trials were performed with the preferred option being HTT for 2 hours at 180° C.

Once the technique had shown to be successful, an attempt was made to switch the base granule from BCP to a β-TCP.

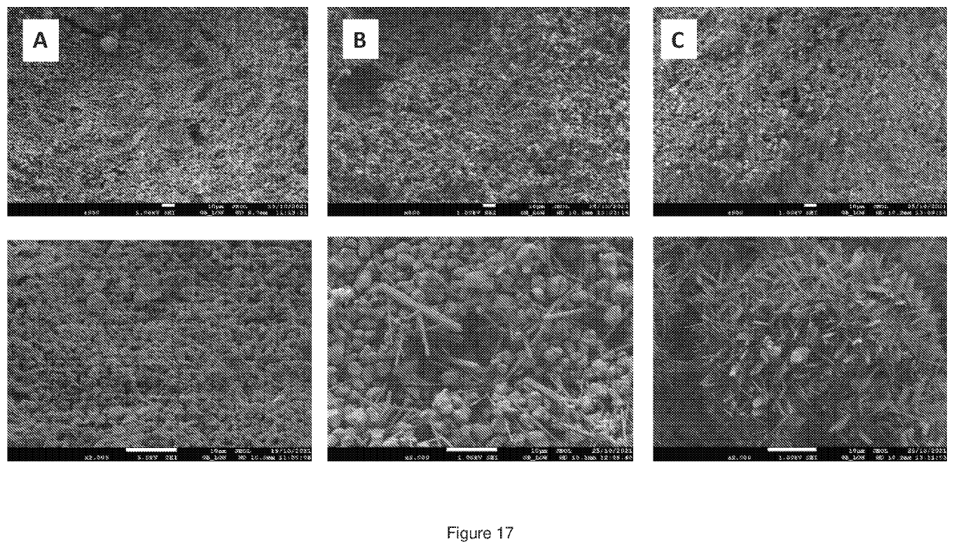

Despite early attempts being unsuccessful, the addition of a small amount of α-TCP into the reactor vessel improved the results (see below). FIG. 17 shows the surface of β-TCP granules having no HTT, HTT for 24 hours at 100° C. and HTT under the same conditions but in the presence of α-TCP. Although some surface needles are formed without α-TCP present, the presence of α-TCP greatly improves the needle formation and coverage across the surface of the granule.

### The Time and Temperature for Optimum HTT

This section briefly describes the studies performed to achieve the optimum time and temperature.

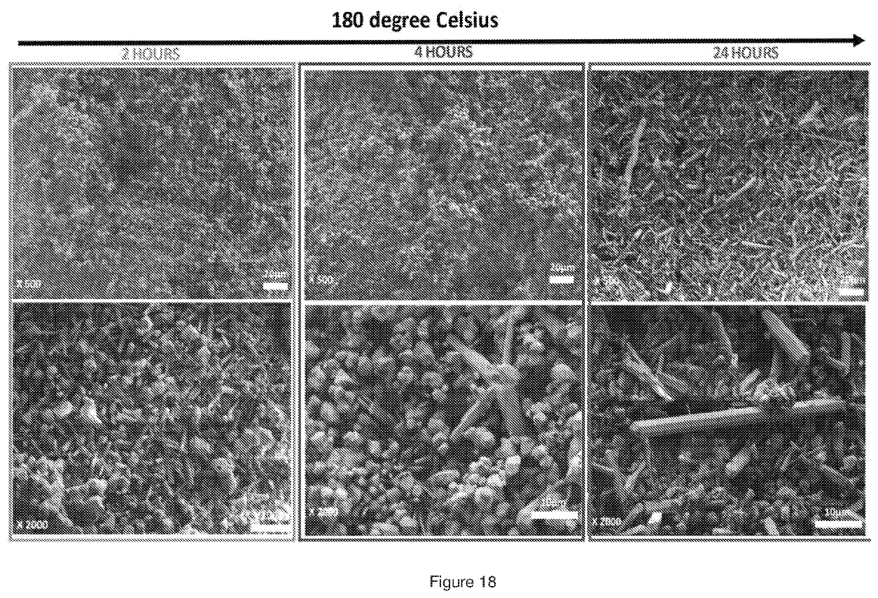

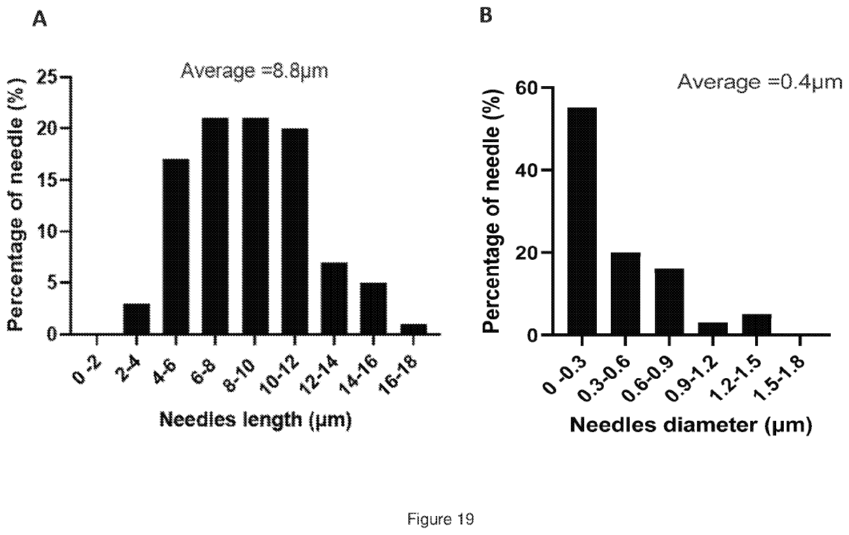

As shown above, the preferred HTT conditions for BCP base granule were 180° C. for 2 hours, but this is not suitable for the β-TCP granule. Longer time points were investigated. At this point no α-TCP was used, but enough needles were formed on the surface to demonstrate that the longer the HTT time, the longer the needles that were formed. This is shown in FIG. 18 where HTT was performed for 2, 4 and 24 hours.

To determine the required needle size, software Image-J and SEM imaging needle size analysis was performed on a competitor product MagnetOs™ and this showed the average needle diameter was 0.4 μm and the average needle length was 8.8 μm (see Comparative FIG. 19).

This gave an indication of the needle size that Certoss™ Granules would need to achieve to be effective and increasing the HTT time at 180° C. resulted in needles that were too large based on the images in FIG. 18.

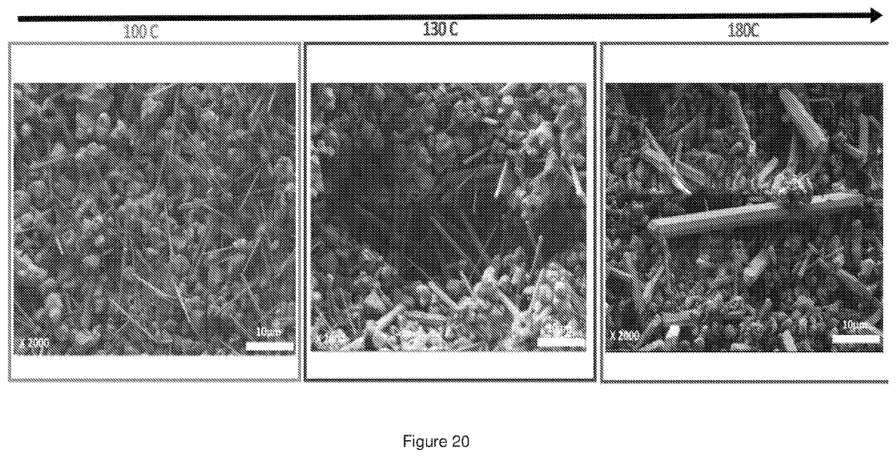

The HTT temperature was now investigated as shown in FIG. 20 and at lower temperatures the surface needles were thinner in diameter and analysis of at least 50 needles showed that they were also shorter in length.

The needle size analysis was performed on a number of test samples and the results are shown in Table 9. The maximum value and minimum value for diameter and length are reported. The average is the mean value. As the SEM show, increasing the HTT time at 180° C. vastly increases needle size. HTT for 24 hours at 100° C. results in a surface needle comparable to that of MagnetOs™ and is the preferred treatment time and temperature.

The addition of α-TCP appears to have little effect on the needle diameter but, potentially reduces the needle length. This was not thought to be a disadvantage relative to the enhanced number and coverage of the needles in the presence of α-TCP.

TABLE 9
Needle size analysis for different HTT'd samples and
MagnetOs ™ (as supplied)

| Material/ | DIAMETER | LENGTH |
|---|---|---|
| Treatment | [min; max] | [min; max] |
| β-TCP | 1 μm | 4.3 μm |
| HTT 2 h | [0.14 μm; 3.2 μm] | [1.6 μm; 11 μm] |
| @180 °C | | |
| β-TCP | 3.7 μm | 18.6 μm |
| HTT 24 h | [0.3 μm; 14 μm] | [1.5 μm; 77 μm] |
| @180 °C | | |
| β-TCP | 0.35 μm | 8.7 μm |
| HTT 24 h | [0.04 μm; 2 μm] | [3.1 μm; 19.5 μm] |
| @100 °C | | |
| β-TCP + α-TCP | 0.46 μm | 4.7 μm |
| HTT 24 h | [0.05 μm; 2.4 | [1. 7 μm; 12.7 μm] |
| @100 °C | μm] | |
| MagnetOs ™ | 0.4 μm | 8.8 μm |
| (batch E1567) | [0.06 μm; 1.48 | [2.88 μm; 17.13 |
| | μm] | μm] |

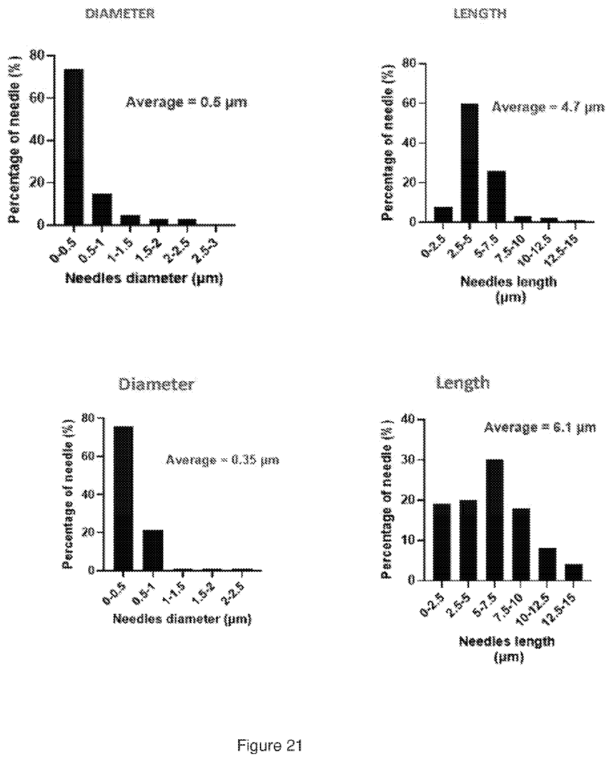

The amount and particle size of the α-TCP in the hydrothermal chamber FIG. 21 shows that the particle size of α-TCP has only a minor effect on the size of the needles formed if kept below 150 μm. SEM images of Certoss™ Granules treated with α-TCP having a particle size of greater than 500 μm resulted in minimal needle growth.

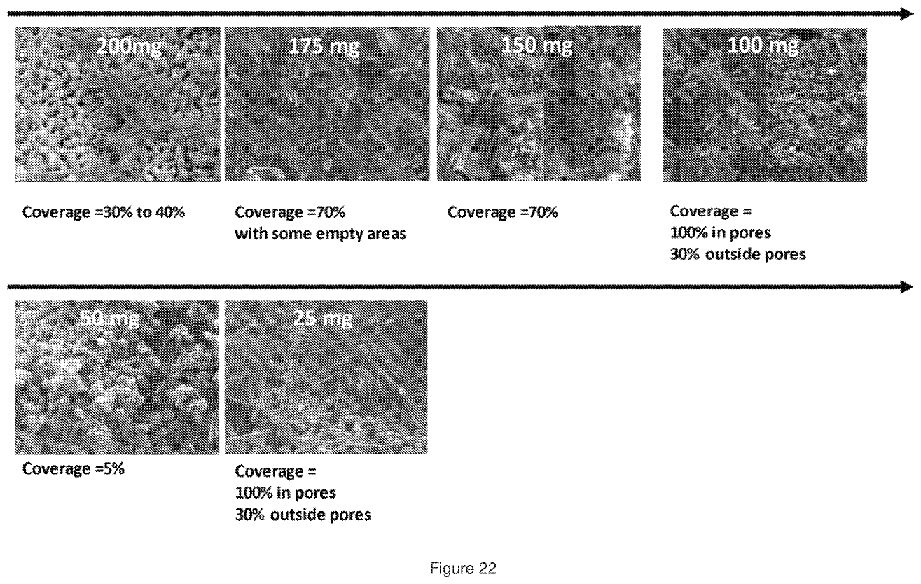

A study was performed to investigate whether the amount of α-TCP in the hydrothermal chamber influenced the needle formation. Representative images are shown in FIG. 22 with an indication of the coverage across the whole surface of the granule. In each case 1 g of β-TCP was used. There was no clear-cut conclusion that could be drawn from this data as the needle coverage did not correlate with the amount of α-TCP present.

### Characterisation Methods

### Protein Adsorption

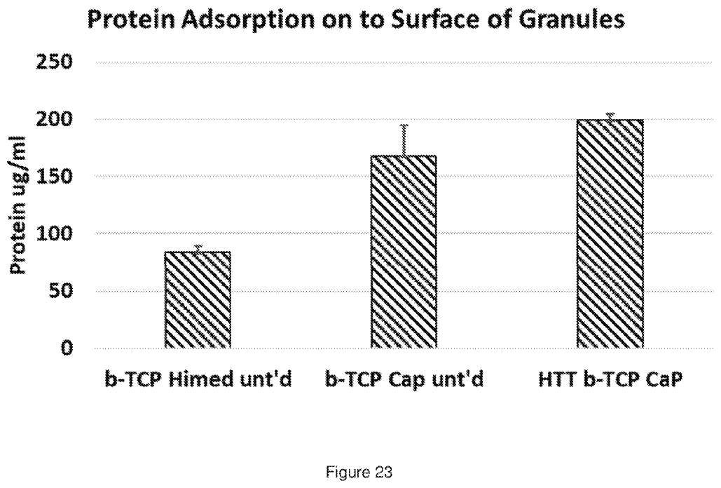

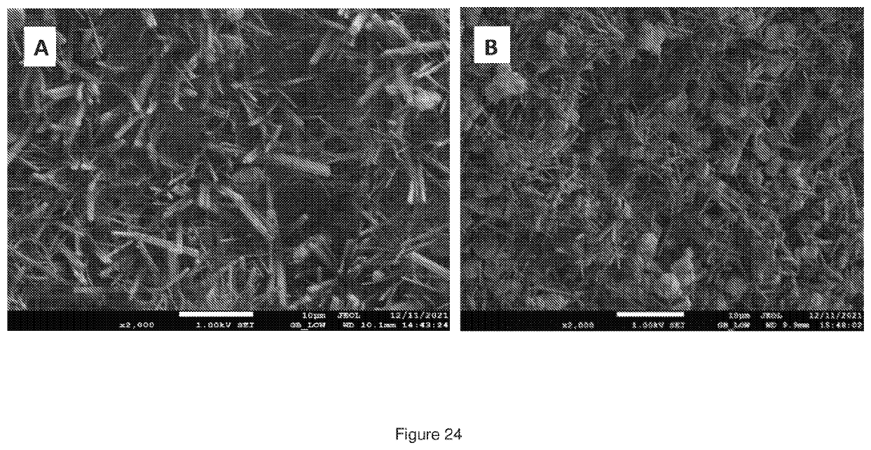

FIG. 23 shows relative amount of protein adsorbed onto the surface of HTT BTCP and untreated β-TCP both supplied by CaP and untreated β-TCP from a different supplier (Himed).

The untreated CaP β-TCP adsorbs more protein when incubated at 37° C. for 24 hours in the presence of complete tissue culture media (DMEM+10% FBS) than a similar material from Himed. Additionally, HTT β-TCP adsorbs more protein than the untreated granules presumably because of the increased surface area due to the needle formation.

### Cell Attachment

It was discovered during the development of Certoss™ Granules that increasing the pH in the reaction vessel resulted in a reduction in needle size. This is shown in the SEM imaging in FIG. 24.

C2C12 cells (mouse myoblast) were seeded onto the granules having different needle sizes to determine which would be the most advantageous.

The cells were seeded at 100,000 onto 100 mg of Certoss™ Granules in an ultra-low attachment tissue culture plate. Metabolic activity could be measured using Presto blue which is non-toxic and allows for multiple readings on the same material.

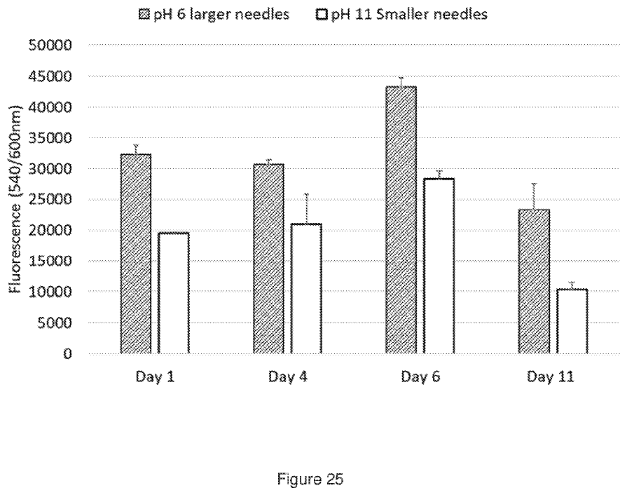

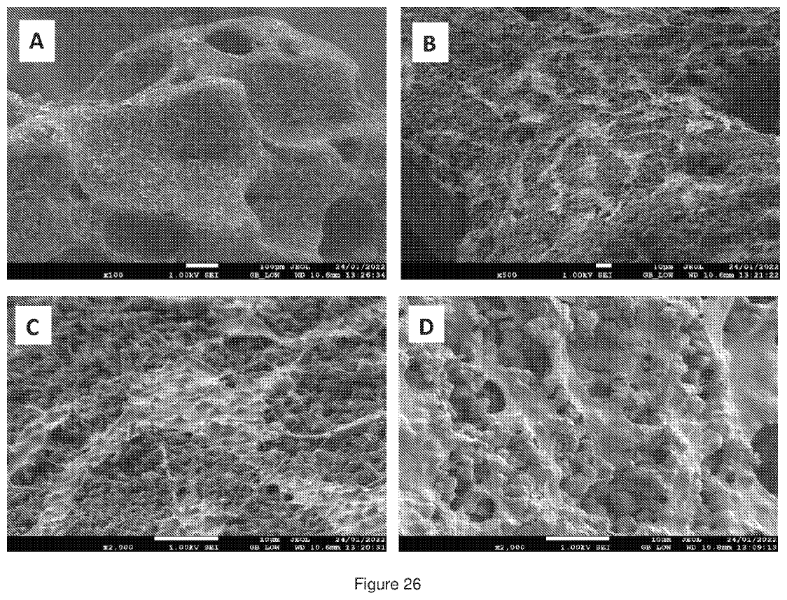

Presto Blue measurements were taken on days 1, 4, 6 and 11 and the data is shown in FIG. 25. From this data it was concluded that the larger needles (of a more similar size the MagnetOs™) were preferable and pH adjustment for HTT was not required. Although cells attached to the material and remained viable, they did not appear to be proliferating on the material.

On day 11 of the above C2C12 cell study, the Certoss™ granules were treated to fix the cells in readiness for SEM imaging. This was done by dehydrating the samples through a series of ascending alcohol baths.

FIG. 26 shows that the cells can be visualized on the granules using this method. Images A, B and C show C2C12 cells on the surface of a HTT Certoss™ Granule at magnifications ×100, ×500 and ×2000. Image D shows the adherence of cells on an untreated β-TCP granule. The cells attached to the β-TCP granules regardless of the HTT.

### Example 3-Comparison of Certoss™ Granules with Predicates

### Key Conclusions

Reprobone™, MagnetOs™ and β-TCP (supplied by CaP Biomaterials) demonstrated the most macroporosity whilst Osteophil, Osteo-3 granules and β-TCP (supplied by Himed) were the least macroporous as determined by light microscopy imaging.

Hydrothermal treatment was successful in altering the surface morphology of β-TCP granules supplied by CaP Biomaterials. The current product name for this material is ‘Certoss™ Granules’.

Only MagnetOs™ and Certoss™ Granules demonstrated surface needles.

Osteo-3 granules adsorbed the most protein to the surface with β-TCP supplied by Himed adsorbing the least. Certoss™ Granules performed better than MagnetOs™.

### Aim

Develop an osteoinductive ceramic granule. Granules purchased ‘off the shelf’ are subjected to a hydrothermal treatment method which encourages the growth of surface needles. This surface treatment is reported to initiate osteoinductivity of the ceramic in-vivo. Treated ceramic granules according to the invention are named ‘Certoss™ Granules’

The company Kuros produce MagnetOs™. The aim of the study was to give an indication of the potential for the Certoss™ Granule to be osteoinductive and to draw comparisons with the materials.

### Summary

A number of different predicates and ‘off the shelf’ ceramic granules were investigated. Firstly, light microscopy was used to determine the gross morphology of each of the ceramic samples. There are clear differences between the test ceramic samples. These relate not only to morphology and surface structure but, also to protein adsorption and cell attachment.

Following on, selected samples were imaged by SEM to investigate the detailed surface structure at high magnifications. This enabled the visualization of surface needles which could then undergo size analysis. There were differences in macroporosity across the test samples. Reprobone™, MagnetOs™ and β-TCP supplied by CaP Biomaterials appeared to be the most macroporous whilst Osteophil, Osteo-3 granules and β-TCP supplied by Himed were the least macroporous as determined by light microscopy.

The granules were tested for their ability to adsorb protein on their surface, and they were also tested to determine whether the surface was suitable for cell attachment.

The Certoss™ Granule uses base granules of β-TCP which are hydrothermally treated to alter their surface by generating micron sized needles. The hydrothermal treatment was successful.

Only MagnetOs™ and Certoss™ Granules demonstrated surface needles at high magnification (SEM). The Certoss™ Granules performed well among the predicates. The needles were evident on the surfaces indicating that the hydrothermal treatment was successful. Protein adsorption was good and better than MagnetOs™. Cells attach well to the optimized Certoss™ granule (FIG. 26).

### Materials

TABLE 10

| Material | Supplier | Cat/Lot Number |
|---|---|---|
| β-Tricalcium Phosphate (1-2 mm) | CaP Biomaterials | TCPDP052120A |
| β-Tricalcium Phosphate (1-2 mm) | Himed | SWB220221 |
| Osteophil beta-TCP (1-2 mm) | Cytophil Inc. | sample |
| MagnetOs ™ (2-4 mm) | Kuros | 703-026 |
| Reprobone ™ (1-4 mm) | Ceramisys | Sample Lot 2110 |
| Osteo-3 Granules (1-2 mm) | SiraKoss | JG105B |
| Certoss ™ Granules (1-2 mm) | Locate Bio Ltd. | Batches 13, 41 & 60 |
| DMEM | Gibco | 21885-025 |
| Fetal Bovine Serum | Gibco | 10500-064 |
| Dulbeccos PBS | Gibco | 14190-144 |
| Penicillin/Streptomycin (PS) | Sigma-Aldrich | P4333 |
| Triton-X | Sigma-Aldrich | T9345 |
| Toluidine blue | Sigma-Aldrich | 88640 |
| Micro BCA kit | ThermoFisher | 23235 |
| C2C12 Cells | ECACC | 91031101 |

The in-house prepared batches of Certoss™ Granules were hydrothermally treated at 180° C. which was the preferred temperature when this predicate comparator work was completed. Batch 13 was treated for 20 hours and batch 41 was treated for the preferred 2 hours in the hydrothermal reactor.

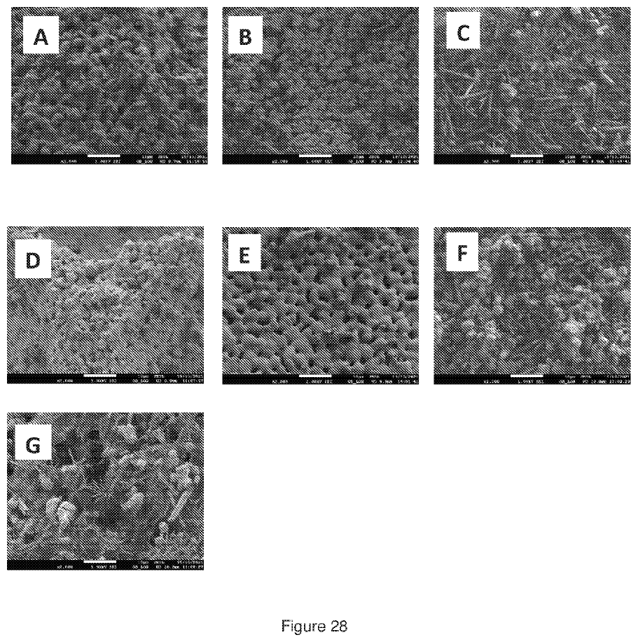

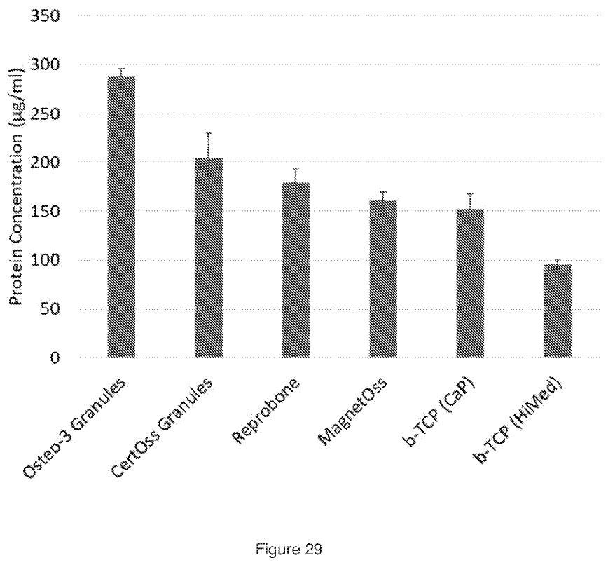

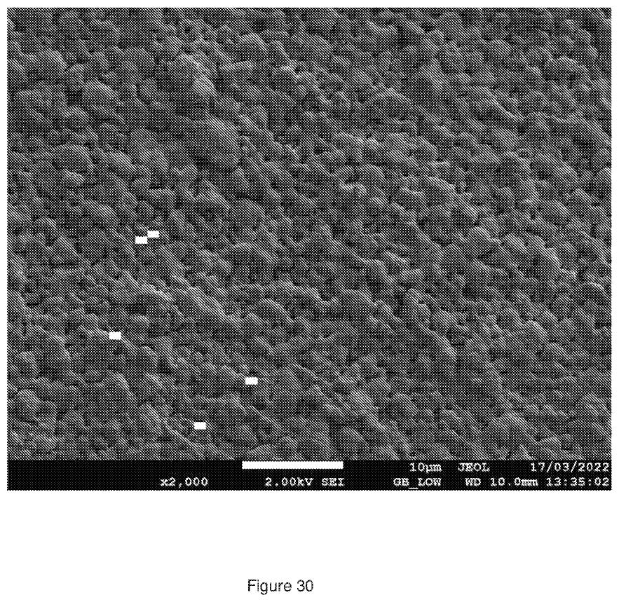

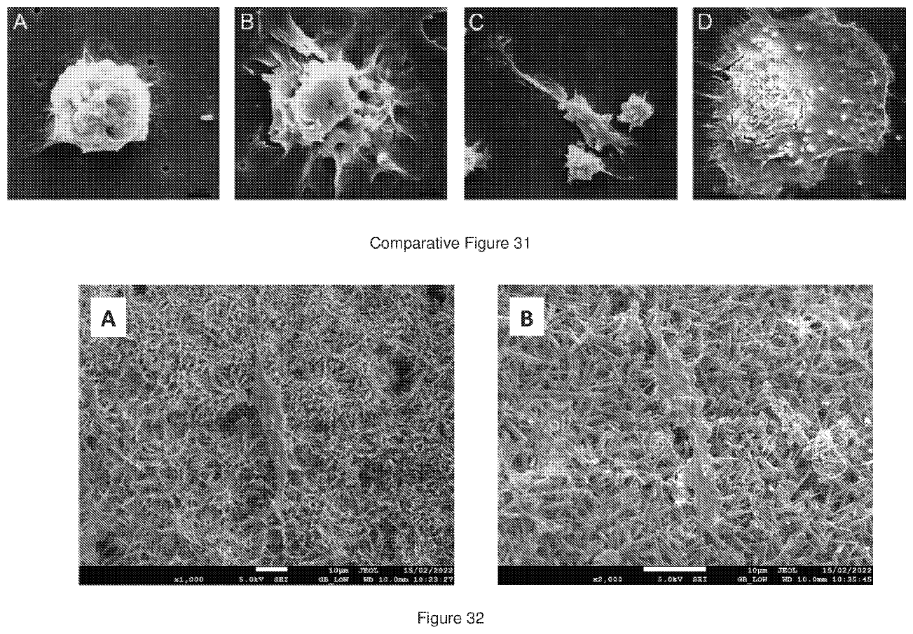

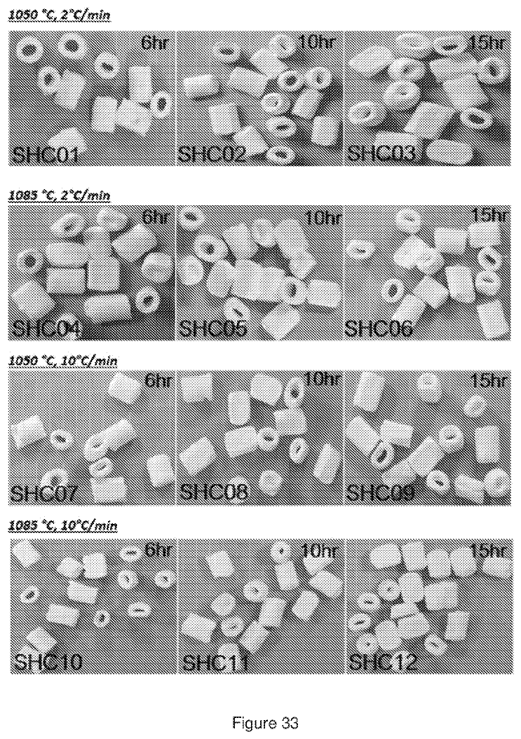

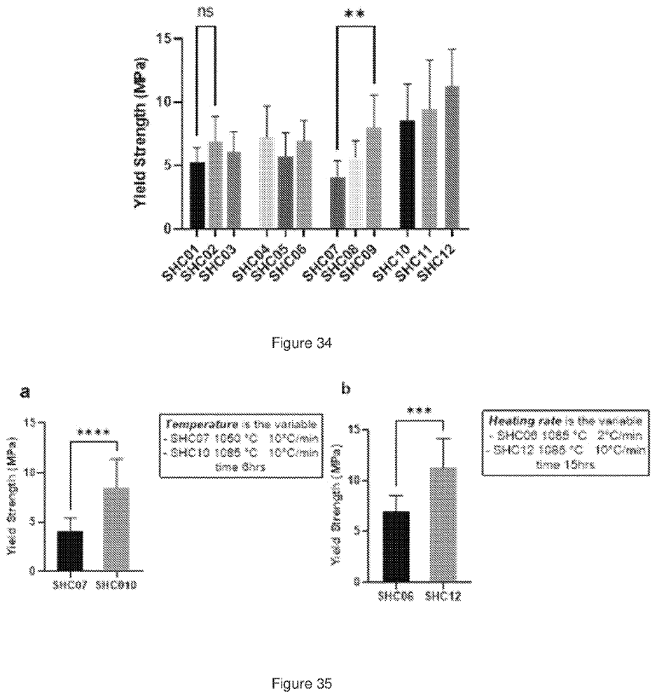

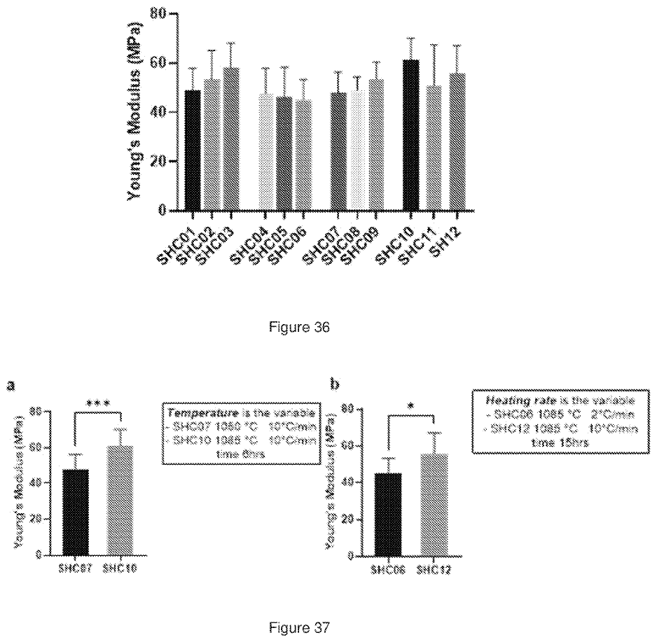

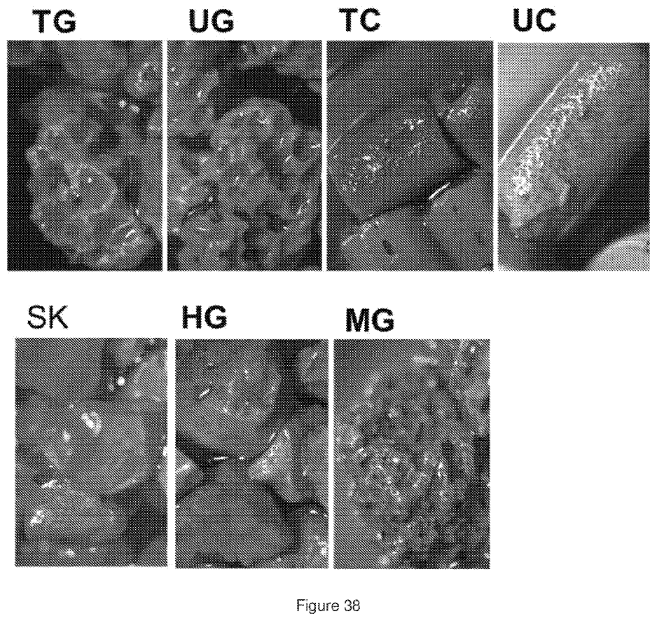

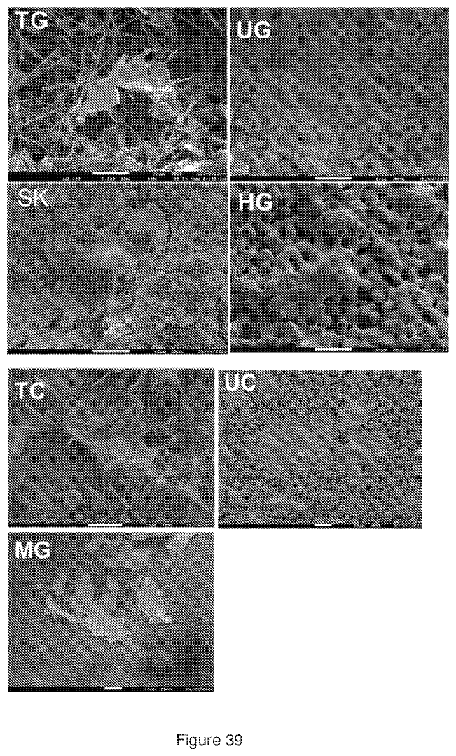

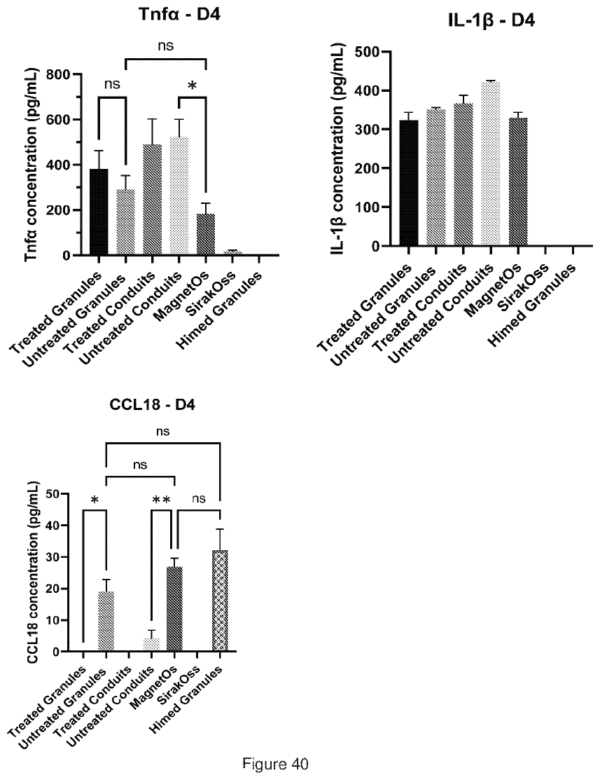

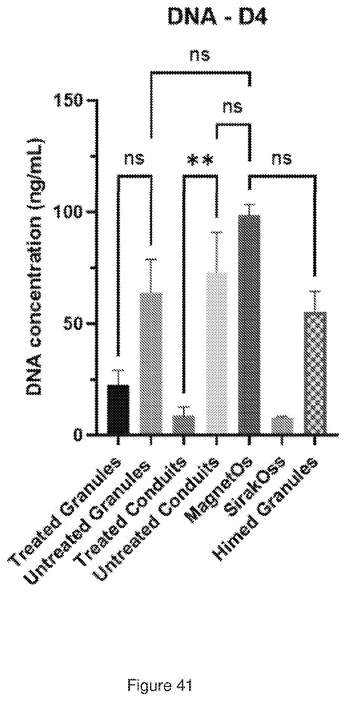

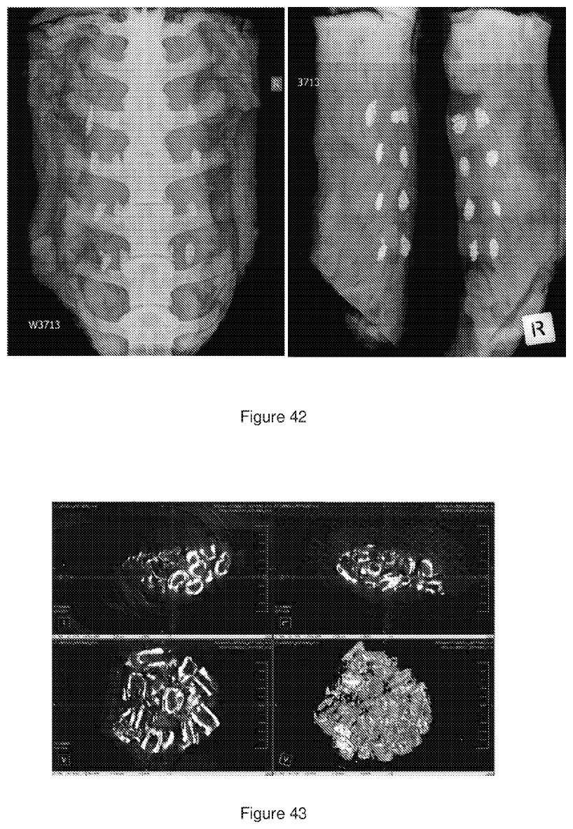

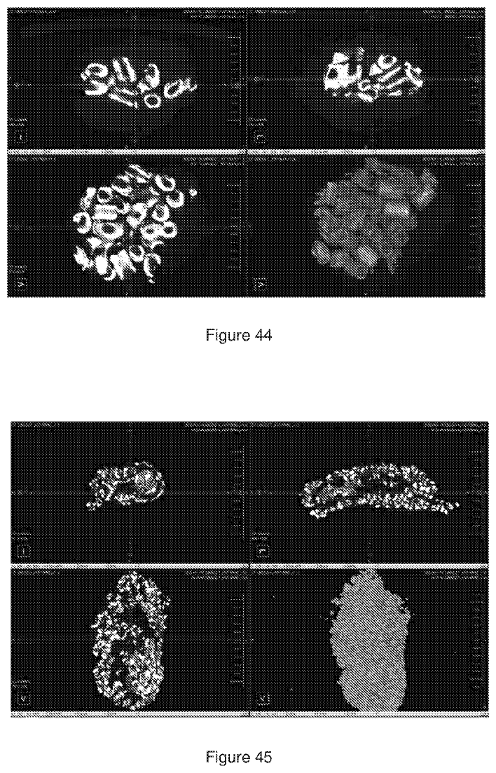

SEM images (see FIG. 28) are also included for batch 60 which was prepared using the improved method of adding α-TCP to the reactor vessel and treating for 20 hours at 100° C. The needle formation of this batch is superior to the previous batches.

### Methods

### Light and Scanning Electron Microscopy (SEM)

For light microscopy, the Brunel SP-28 Microscope and Canon 1100D digital camera was used. The images were taken at 1× magnification with a 10× objective lens. The materials were placed in a black weigh boat for imaging to maximize the contrast. Once the materials were mounted on an appropriate stub, the Quorum Q150R ES sputter coater was used to coat them with a 10 nm gold coat.

The samples were imaged using the JEOL JSM-7100FLV scanning electron microscope using an acceleration voltage of 5 Kv. In each case a few different granules were inspected to ensure they were all similar. Images were taken at different magnifications. The 2000× magnification images are shown. See FIG. 28.

### Needle Size Analysis

The image analysis software Image-J was used to determine the size of the needles for some of the materials. This software can be calibrated using the image scale bar and can then measure needle length and diameter by the user manually drawing onto the needles.

The data is plotted as a size range bar chart.

### Protein Adsorption

100 mg of each ceramic sample was weighed directly into a well of a 24 well tissue culture plate. 2 ml of complete tissue culture media (DMEM+10% FBS+PS) was added to each well containing samples and media alone controls were also prepared to ensure protein was not adhering to the plate. The plate was incubated for 24 hrs at 37° C./5% CO₂ then, all the samples were thoroughly washed 3× with 2 ml of DPBS. A 1% v/v solution of Triton-X was added to the wells (0.5 ml) and the plate was left for 30 minutes at room temperature on a plate shaker set at minimum rpm to gently agitate.

An assessment of the protein concentration in each supernatant was determined by following the manufacturer's instructions for the ThermoFisher Micro BCA kit. A solution of 1% v/v Triton-X was used to dilute the calibration standards.

### Results

### Needle Size Analysis

FIG. 28 shows SEM images of seven different ceramic samples at ×2000. All surfaces appear to be microporous but only MagnetOs™ and Certoss™ Granules show needles on the surface.

Needle size analysis was performed on Certoss™ Granules (Batch 41) and MagnetOs™. The Certoss™ Granules needles were on average shorter (4.3 μm vs 8.8 μm) and thicker (0.4 μm vs 1 μm) when compared to MagnetOs™

### Protein Adsorption

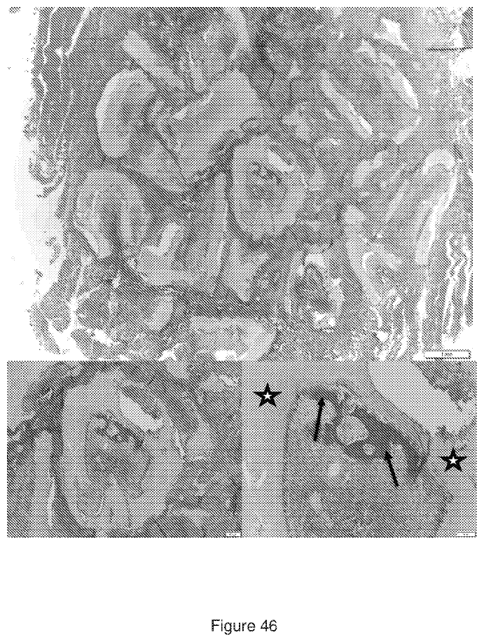

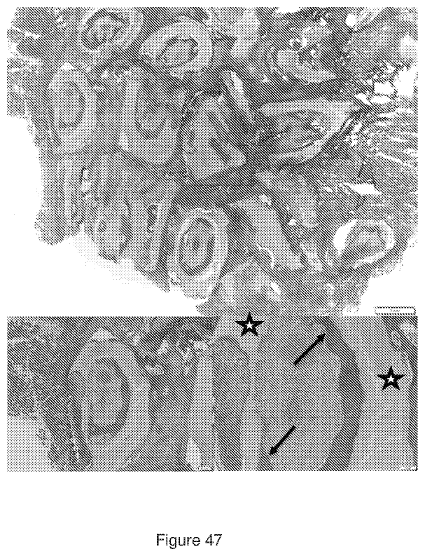

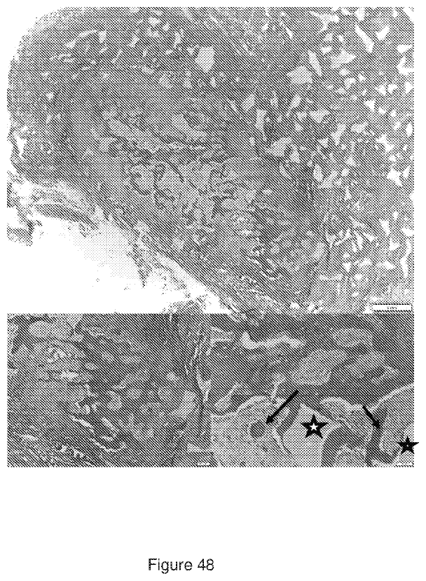

FIG. 29 shows the amount of protein adsorbed to the surface after 24 hours in the presence of 10% FBS in culture media. Certoss™ Granules performed well second only to Osteo-3 granules. MagnetOs™ did not perform as well as Certoss™ Granules in this study. Certoss™ Granules also performed better than the base β-TCP granule from CaP.

The Certoss™ Granules for this study were batch 13.

They can be ranked from having least to most protein adsorption as follows:

| | | |
|---|---|---|
| Least Protein | [symbol] | Most Protein |

| | | | | | |
|---|---|---|---|---|---|
| β-TCP | β-TCP | MagnetOs ™ | Reprobone ™ | Certoss ™ | Osteo-3 |
| (Himed) | TCP (CaP) | | | Granules | Granules |

### Example 4: Investigating the mechanical strength of Certoss™ Conduit

### Aims and Background

Investigating which factors directly influence the mechanical strength of the sintered conduits. The dependent variables which thought to impact the mechanical stiffness of the conduits were: the temperature, the heating rate, and the sintering time. The aim was to assess whether stronger conduits could be made without losing shape and lumen circularity.

### Methodology

Certoss™ conduits were sintered following the condition laid out in Table 11. The SEG caption, refers to the segment session in the program of the furnace used for sintering. The heating rate of SEG-1 and the dwell time of SEG-2 have been kept constant for all sintering conditions, as this first step only burns off the ELVAX™ polymer.

Once all the sintering trials have been completed, between 10-20 individual conduits were selected for compressive strength characterization. The conduits compressive strength was tested using the Texture Analyzer with a test speed of 0.04 mm/sec as described in ISO 5833, and a strain of 50%. Stress-strain graphs were recorded as well as the Yield Strength and the conduit Young's Modulus (MPa).

TABLE 11
Sintering conditions used for the manufacture of
Certoss ™ conduits for the mechanical stability testing.

| | SEG-1 | SEG-2 | SEG-3 | SEG-4 | SEG-5 |
|---|---|---|---|---|---|
| SHC01 | Rate-500 | Dwell- | Rate-1050 | Dwell-6 hrs | End |
| | °C, 2 °C/min | 3 hrs | °C, 2 °C/min | | |
| SHC02 | Rate-500 | Dwell- | Rate-1050 | Dwell-10 hrs | End |
| | °C, 2 °C/min | 3 hrs | °C, 2 °C/min | | |
| SHC03 | Rate-500 | Dwell- | Rate-1050 | Dwell-15 hrs | End |
| | °C, 2 °C/min | 3 hrs | °C, 2 °C/min | | |
| SHC04 | Rate-500 | Dwell- | Rate-1085 | Dwell-6 hrs | End |
| | °C, 2 °C/min | 3 hrs | °C, 2 °C/min | | |
| SHC05 | Rate-500 | Dwell- | Rate-1085 | Dwell-10 hrs | End |
| | °C, 2 °C/min | 3 hrs | °C, 2 °C/min | | |
| SHC06 | Rate-500 | Dwell- | Rate-1085 | Dwell-15 hrs | End |
| | °C, 2 °C/min | 3 hrs | °C, 2 °C/min | | |
| SHC07 | Rate-500 | Dwell- | Rate-1050 | Dwell-6 hrs | End |
| | °C, 2 °C/min | 3 hrs | C. 10 °C/min | | |
| SHC08 | Rate-500 | Dwell- | Rate-1050 | Dwell-10 hrs | End |
| | °C, 2 °C/min | 3 hrs | °C, 10 °C/min | | |
| SHC09 | Rate-500 | Dwell- | Rate-1050 | Dwell-15 hrs | End |
| | ºC, 2 °C/min | 3 hrs | °C, 10 °C/min | | |
| SHC10 | Rate-500 | Dwell- | Rate-1085 | Dwell-6 hrs | End |
| | °C, 2 °C/min | 3 hrs | °C, 10 °C/min | | |
| SHC11 | Rate-500 | Dwell- | Rate-1085 | Dwell-10 hrs | End |
| | °C, 2 °C/min | 3 hrs | °C, 10 °C/min | | |
| SHC12 | Rate-500 | Dwell- | Rate-1085 | Dwell-15 hrs | End |
| | °C, 2 °C/min | 3 hrs | °C, 10 °C/min | | |

### Results

From the images of each sintering condition shown in FIG. 33, it appears that the longer the sintering time the flatter the conduit appear. Also, the conduit seemed to have lost the hollow characteristic and lumen circularity.

Furthermore, the higher the heating rate the more shrunken and flattened the lumen appeared.

The results demonstrate:
A sintering time greater than 10 hours resulted in loss of lumen circularity.
A sintering temperature >1050° C. resulted in flattened shape conduits and loss of lumen.
A fast heating rate (10° C./min) resulted in conduits that appeared shrunken and flattened.
Sintering time has no impact on the conduits strength.

### Optimum Conditions

6-10 hours sintering time, heating rate<10° C./min and a sintering temperature<or equal to 1050° C.

### Yield Strength-Sintering Time

FIG. 34. Yield strength of conduits. Sintering time appear not to have an effect on the Yield strength of the conduits except for SHC07 and SHC09. Where at a higher heating rate (10° C./min), the longer the sintering time, the higher the yield strength.

### Dependent Variables

### Temperature

At the early sintering time of 6 hours, the higher the temperature the higher the Yield Strength.

With a constant heating rate of 10° C./min the Yield Strength is higher when the sintering temperature is 1085° C. No significant difference between the Yield strength and the sintering time for each condition.

### Heating Rate

At constant sintering temperature of 1050° C., at 15 hours yield strength of SHC09 is significantly higher than SHC03. However, for 6 hour and 10 hours the trend seems to be the opposite.

At constant sintering temperature of 1085° C., the 10 and 15 hour yield strength of SHC11 and SHC12 are significantly higher than SHC05 and SHC06, respectively.

FIG. 35. Representative graphs of Yield strength between sintered conduits group where in a. Temperature is the dependent variable and in b. heating rate is the dependent variable.

### Young's Modulus—Sintering Time

FIG. 36. Sintering time has no impact on conduits Young's modulus at each sintered conditions stated in the graphs.

### Dependent Variables

### Temperature

At constant heating rate of 10° C./min, Young's Modulus values significantly higher for SHC10 temperature compared to SHC07.

### Heating Rate

Heating rate as no impact on conduits Young's modulus at 1050° C. sintering temperature.

Heating rate at 1085° C. sintering temperature, seems to have an impact on Conduits Young's modulus at 6 and 15 hrs. Higher heating rate higher Young's modulus values.

FIG. 37. Representative graphs of Young's Modulus between sintered conduits group where in a. Temperature is the dependent variable and in b. heating rate is the dependent variable.

### Example 5: Macrophage Polarization and Differentiation on Certoss™ Products and Predicates

### Aims and Background

To differentiate the THP-1 cell line into M0 macrophage and polarized them on Certoss™ Granules, Conduits and Predicates. Once THP-1 differentiated to M0 they become adherent cells. The objective was to investigate whether the surface topography of the ceramic materials (with ‘hair’, treated granules and conduits) will be enough to induce the polarization of M0 macrophage towards an M1 (pro-inflammatory) or an M2 (anti-inflammatory) phenotype. To identify whether the cells have differentiated, culture media was assessed for Cytokines specific of M1 and M2 phenotype with 4 ELISA Kits. Cells were also fixed for SEM imaging to analyse cell morphology.

### Methodology

Firstly, THP-1 cell line purchased from ATCC was expanded until a bulk stock of master vials was created. A working stock of THP-1 was expanded until the desired cell number was reached for the initiation of the experiment.

400,000 cells/well were added on top of Certoss™ Granules, Conduits and Predicates as shown in Table 12. THP-1 were differentiated to M0 directly onto the ceramics using PMA for 48 hours. After differentiation to M0, Positive controls were treated with the respective growth factors for M1 and M2 phenotype, while the rest of the M0 polarized cells on ceramics were given normal medium without growth factors. Cytokines release, from ELISAs assay, TNFα and IL-1β, show that macrophages on ceramics have been differentiated towards an M1 pro-inflammatory phenotype. SEM was used to image the cells onto the ceramics. Picogreen Assay was used to determine the DNA content at D4 and for data normalization.

TABLE 12
TCP ceramic samples used to study macrophage differentiation.

| | Sample | | |
|---|---|---|---|
| Sample Name | ID | Supplier | Lot |
| Treated Granules | TG | CaP | 220702 |
| Untreated Granules | UG | CaP | TCPDP052120A (1-2 mm) |
| Treated Conduits | TC | CaP | 220801 |
| Untreated Conduits | UC | CaP | 220801 |
| SiraKoss | SK | SiraKoss | JG105SB (ii) (1-2 mm) |
| Himed Granules | HG | Himed | SWB220221 (1-2 mm) |
| MagnetOs ™ | MG | Kuros | E1567 (2-4 mm) |
| | | Biosciences | |

FIG. 38: Toluidine blue staining of macrophages differentiated onto ceramics granules, conduits and predicates. A lot of cells on HG, UC and MG. Cells were situated in the concavities of the granules.

FIG. 39: Representative SEM images of differentiated macrophages on Certoss™ conduits, granules, and predicates.

FIG. 40: (a), (b) Pro-inflammatory citokines TNFα and IL-1β expressed in differentiated macrophages on ceramic materials at D4. (c) Anti-inflammatory cytokine CCL18 expressed in differentiated macrophages on ceramic materials at D4.

High expression of TNFα at D2, with a reduction at D4. Levels of expression for IL-β are similar across all ceramic groups except for SK and HG. Not many cells were found on SK granules whereas HG granules seems to have differentiated the macrophages towards an anti-inflammatory phenotype as CCI18 is highly expressed. However, relative DNA content suggests not many cells attached on SK granules.

FIG. 41: DNA concentration of differentiated macrophages retrieved from ceramics materials.

It appears an equivalent amount of DNA was retrieved from Untreated granules, untreated conduits, and MagnetOs™. However significant less DNA was retrieved from treated granules and conduits, suggesting that cells might have not liked the ‘needle-like’ surface topography of the treated ceramics.

### Conclusion

Cytokines, TNFα and IL-1β, show that macrophages on ceramics have been differentiated towards an M1 pro-inflammatory phenotype.

M2 cytokine CCL18 was slightly expressed in untreated granules, untreated conduits, Himed granules and MagnetOs™.

It was not possible to clearly identify the phenotype of the macrophage cultured on the ceramics through SEM imaging.

DNA retrieved from Positive control samples suggest more cells were present in the control than the tested samples, indicating that some cells did not attach onto the ceramic materials.

### Example 6: Ovine Intramuscular Screening

### Aims and Background

Designed to evaluate the in vivo response of bone graft substitute materials when implanted in an intramuscular defect model in adult sheep at 6 and 12 weeks.

The specific aims in an intramuscular site were:
1. To evaluate the radiographic appearance. Faxitron radiographs and Micro CT.
2. To evaluate the local tissue reactions based on paraffin histology.

### Methodology:

1a. Faxitron Radiographs

The harvested samples were radiographed using a Faxitron and digital plates (AGFA CR MD4.0 Cassette) (settings 32 kV for 25 seconds) as per the SORL Faxitron protocol. The Faxitron radiographs in the posteroanterior planes was assessed for evidence of adverse reactions in a blinded fashion by the facility manager and study director.

1b. Micro CT

Micro Computed tomography (uCT) was performed on the muscle samples using an Inveon Scanner (Siemens, USA). Slice thickness was set to approximately 50 microns for all scans. CT scans were be stored in DICOM format. Three dimensional models were reconstructed and examined in the axial, sagittal, and coronal planes.

Axial, sagittal, and coronal images are provided for each site. The micro-CT reconstructions were evaluated by reviewing the coronal and sagittal planes to assess for any signs of new bone formation at 6 and 12 weeks.

2a. Histology

The samples were decalcified in 10% formic acid-phosphate buffered formalin at room temperature. Samples were decalcified in this solution 3-4 days prior to gross sectioning into paraffin cassettes.

The decalcified samples were sectioned into two pieces. The cut sections were placed into embedding blocks for paraffin processing. Each paraffin block was sectioned (5 microns) using a Leica Microtome and placed on slides for haemotoxylin and eosin (H&E). Staining was performed according to SOP-11: Standard Operating Procedure: H&E Staining.

Stained sections were examined under light microscopy using an Olympus Microscope with an Olympus DP72 high resolution video camera to capture images. The reviewer was blinded to time points and treatment groups. Histology was qualitatively assessed at each time point and a summary written.

### Results

Radiographs-High-definition radiographs were taken in the posteroanterior (PA) plane using a Faxitron and digital plates (AGFA CR MD4.0 Cassette. No evidence of adverse reactions was noted.

FIG. 42: Example Faxitron radiographs with the spine (left) and after muscle harvest (right) at 6 weeks. Differentiation of new bone and residual material is not possible based on this endpoint. No adverse reactions were noted. All materials were present.

Micro CT-Micro-CT at 6 and 12 weeks was performed to assess the osteoinductivity of the test and predicate materials and to look for evidence of any adverse reactions. No adverse reactions were noted on the micro-CT images.

FIG. 43: Example of Micro-CT screen shot for group 2 at 12 weeks (3715 LM3). New bone formation was noted on examination of the Micro-CT data (arrow).

### Group 4-Conduit Non-HTT

FIG. 44: Example of Micro-CT screen shot for group 4 at 12 weeks (3715 LL4). New bone formation was noted on examination of the Micro-CT data (arrow).

### Group 5-Attrax (Predicate)

FIG. 45: Example of Micro-CT screen shot for group 5 at 12 weeks (3715 RM2). New bone formation was noted on examination of the Micro-CT data (arrow).

Histology-All samples were osteoinductive at 12 weeks, while at 6 weeks only group 4 (Conduit non-HTT) and group 5 (Attrax) had all the samples confirmed as osteoinductive.

### Group 2-Conduit HTT

FIG. 46: Example of paraffin histology at 12 weeks for Group 2-(3715 LM3). The implanted material has begun to resorb but still present (star). New bone (black arrow) was noted on and around the material. No inflammatory cells are present.

### Group 4-Conduit Non-HTT

FIG. 47: Example of paraffin histology at 12 weeks for Group 4-(3715 LL4). The implanted material has begun to resorb but still present (star). New bone (black arrow) was noted on and around the material. No inflammatory cells are present.

### Group 5-Attrax (Predicate)

FIG. 48: Example of paraffin histology at 12 weeks for Group 5-(3715 LL4). The implanted material has begun to resorb but still present (star). New bone (black arrow) was noted on and around the material. No inflammatory cells are present.

### Effect of HTT on Total Pore Area

The tables 13 and 14 below show total pore areas (m²/g), when HTT treatment is used. Values were measured using a mercury porosimeter. In both cases of high and low needle coverage the total pore area increased.

TABLE 13

| | Total intrusion | Total Pore | Average pore | Bulk | Apparent skeletal | |
|---|---|---|---|---|---|---|
| Sample | Volume | area | diameter | Density | density | |
| ID | (mL/g) | (m²/g) | (μm) | (g/mL) | (g/mL) | Porosity |
| HTT Granules | 0.78 | 1.92 | 1.62 | 0.853 | 2.51 | 66.08% |
| (high coverage) | | | | | | |
| Granules | 0.73 | 0.76 | 3.83 | 0.884 | 2.49 | 64.52% |
| HTT Conduits | 0.34 | 0.94 | 1.47 | 1.435 | 2.83 | 49.25% |
| (low coverage) | | | | | | |
| Conduit | 0.29 | 0.76 | 1.54 | 1.566 | 2.89 | 45.75% |
| SiraKoss | 1.31 | 37.41 | 0.140 | 0.597 | 2.72 | 78.00% |

Further analysis of mercury intrusion (removed the inter-particulate contribution)−(m2/g does not change but the way porosity is calculated has changed):

TABLE 14

| | Total Pore | Average pore | |
|---|---|---|---|
| Sample ID | area (m²/g) | diameter (μm) | Porosity |
| HTT Granules | 1.90 | 0.787 | 48.46% |
| (high coverage) | | | |
| Granules | 0.750 | 1.535 | 41.78% |
| HTT Conduits | 0.934 | 1.199 | 44.19% |
| (low coverage) | | | |
| Conduit | 0.760 | 1.411 | 43.63% |
| SiraKoss | 37.403 | 0.124 | 75.82% |

### Conclusion

This study evaluated the in-vivo response, at 6 and 12 weeks of four different test materials and one predicate material in an intramuscular defect model in adult sheep. The materials evaluated were Group 1-Granules HTT, Group 2-Conduit HTT, Group 3-Granules non-HTT, Group 4-Conduit non-HTT, and Group 5-Attrax (predicate). Endpoints included Faxitron radiographs, Micro-CT, and paraffin histology.

The implantation of materials was uneventful and was achieved in a reproducible manner. Surgery was completed without any adverse events. All animals recovered well immediately following surgery. No adverse reactions were noted during harvest.

High resolution Faxitron radiographs showed no evidence of adverse reactions, and all materials were visualized and were implanted in the correct anatomical position. No adverse reactions were noted on the micro-CT images. Some bone was notable in same images, but it was difficult to discern newly formed bone to the implanted materials therefore osteoinductivity assessment was done on histology images. Based on histology, all samples were osteoinductive at 12 weeks, while at 6 weeks only group 4 (Conduit non-HTT) and group 5 (Attrax) had all the samples confirmed as osteoinductive.

### Terminology

HTT: Hydrothermal Treatment
BCP: Bi-phasic Calcium Phosphate
β-TCP: Beta-Tricalcium Phosphate
α-TCP: Alpha-Tricalcium Phosphate
α-TCP and β-TCP are two different crystalline forms of tricalcium phosphate (a calcium salt of phosphoric acid, Ca₃(PO₄)₂). Both forms α-TCP and β-TCP are available commercially.
HME: Hot Melt Extrusion
CaP: CaP Biomaterials (Supplier)
Certoss™ Granules: HTT of β-TCP Granules
Certoss™ Conduit: β-TCP powder with polymer (ELVAX™ polymer used in
Examples) post-HME and post-sintering.
HA: Hydroxyapatite
SEM: Scanning Electron Microscopy
FBS: Fetal Bovine Serum
CaP: CaP Biomaterials (Supplier)
DMEM: Dulbeccos modification of Eagle's medium.
FBS: Fetal bovine serum
PS: Penicillin/streptomycin
BCA: Bicinchoninic acid
SEM: Scanning electron microscopy

### REFERENCES

### Reference LOCSOP124.01

The method below is for one 100 ml hydrothermal reactor. Volumes and masses may change when performing investigational work.
Pre-heat oven to 100° C.
Weigh out 1 g of β-TCP granules (1-2 mm).
Weigh out 0.2 g of α-TCP particles (Himed milled to below 150 μm) or Plasma Biotal as supplied.
Mix together and add to PTFE liner of a 100 ml reactor vessel
Add 50 ml of deionised water
Add lid to PTFE liner
Place liner into reaction vessel and seal.
Place hydrothermal reactor into 100° coven (wear thermal gloves)
Leave for 20-24 hours
Swirl the reactor using a thermal gloved hand at least three times during the HTT process.
Remove from oven (wear thermal gloves) and leave reactors to cools.
Once cooled to approx. 40° C. the reaction vessels can be opened and the PTFE liner removed.
Water can be carefully poured out of the PTFE liner, then the liner is placed, lid off, into a 60° C. oven and left for 2 hours.
The treated granules are transferred to a crucible and placed into a furnace.
The furnace is set to ramp up at 2° C. per minute to 500° C. then to hold the temperature for 3 hours before returning to room temperature.
The CertOss Granules can be removed from the crucible and placed in a labelled container for storage.

### Reference LOCSOP125.01

### Milling ELVAX™ M250

ELVAX™ 250 is supplied as beads which require size reduction for HME. Transfer approx. 50 g of ELVAX™250 to a stainless-steel ladle then, slowly and carefully lower into a small cannister of liquid nitrogen.
Leave for at least 10 minutes.
Set the Fritsch Pulverisette to 10,000 rpm for 120 seconds.
Transfer the beads to the milling vessel and allow excess liquid nitrogen to dissipate.
Add the lid to the vessel, close the cover and start milling.
Retrieve the sample and place on a 1 mm woven mesh sieve. Collect the sieved fraction.
ELVAX™250 is very difficult to mill and this procedure may need repeating 2/3 times to get a sufficient amount of material.

### Feedstock Manufacture

Prepare a small (5 g) ‘pre-feedstock’ for HME by adding 2.6 g milled ELVAX™ M250 to 2.5 g of β-TCP powder. Manually mix then place of a rotator for 5 minutes.
Weigh out the required mass of β-TOP powder and milled ELVAX™250 to enable the β-TCP to have an 80% (w/w) content. Geometrically mix the two components together in the correct ratio e.g. 8 g of β-TCP and 2 g of milled ELVAX™250 on a rotator for 5 minutes. Then, add another 8/2 g and mix again. Repeat until all weighed material is mixed.
Both feedstocks are now prepared.

### Hot Melt Extrusion

The ThreeTec HME is set up with the following parameters:
Hopper feed set initially at 4%-Temperature set at 90, 130, 130 130° C.
Screw speed set at 15 rpm
Pelletiser set at 0.39 m/min pull and 3 mm cut
The pelletiser needs to be set as close as possible to the conveyor and the conveyor set as close as possible to the die (warning hot).
The pre-feedstock is placed into the hopper and the feeder initiated. Once material falls onto the screws, the HME screws are started.
When the hopper is empty, the main feedstock can be added (just 10-20 g at a time).
The initial extrudate is white in colour and ‘stretchy’. This material can be discarded, the purpose of this material is to avoid a build-up of compacted powder within the die.
It is obvious when the main feedstock begins to extrude as the extrudate is grey in colour and breaks easily.
The feeder should be slowed down to 1.6% and the screws can be slowed as well if required. The feed rate and screw speed needs constant monitoring and can be adjusted as the Operator sees fit to avoid build up on the screws.
The conveyor is started at a nominal minimum speed and the extrudate gently pulled towards the pelletiser.
The extrudate can be fed directly into the pelletiser but, it is acceptable (and avoids any squashing of the strand) to snap off the extrudate, allow it to cool for a few minutes and then feed the strands directly in the pelletiser.
The settings on the pelletiser can be adjusted to ensure pellets with the correct aspect ratio are achieved. The target aspect ratio is 1.5 to 2 (ideal dimensions 3.5 mm long, 2 mm in diameter).

### Sintering Pellets

The pellets from the HME process require sintering to convert to untreated CertOss Conduit pellets. This is performed in the Carbolite Gero 12/5 Chamber Furnace utilising a two-step sintering process.
Approximately 1 g of HME generated Conduit pellets are transferred to a crucible.
Currently, up to 6 crucibles can be placed in the furnace at one time.
The furnace is set to ramp up at 2° C. per minute to 500° C. then to hold the temperature for 3 hours. A further ramp up at 2° C. per minute to 1050° C. is performed which is held for 6 hours before the furnace is allowed to cool.
The pellets are retrieved from the furnace, placed into a container. Gentle shaking will break up the majority of those sintered together.

### Hydrothermal Treatment

The hydrothermal treatment is performed in the same way as for CertOss Granules (see Reference LOCSOP124.01) but with half the amount of α-TCP.

The method below is for one 100 ml hydrothermal reactor. Volumes and masses may change when performing investigational work.
Pre-heat oven to 100° C.
Weigh out 1 g of CertOss Conduit.
Weigh out 0.1 g of α-TOP particles as supplied (Plasma Biota!).
Mix together and add to PTFE liner of a 100 ml reactor vessel.
Add 50 ml of deionised water
Add lid to PTFE liner
Place liner into reaction vessel and seal.
Place hydrothermal reactor into 100° C. oven (wear thermal gloves)
Leave for 20-24 hours
Swirl the reactor using a thermal gloved hand at least three times during the HTT process.
Remove from oven (wear thermal gloves) and leave reactors to cool.
Once cooled to approx. 40° C. the reaction vessels can be opened and the PTFE liner removed.
Water can be carefully poured out of the PTFE liner, then the liner is placed, lid off, into a 60° C. oven and left for 2 hours.
The treated Conduit pellets are transferred to a crucible and placed into a furnace.
The furnace is set to ramp up at 2° C. per minute to 500° C. then to hold the temperature for 3 hours before returning to room temperature.
The CertOss Conduit pellets can be removed from the crucible and placed in a labelled container for storage.

### Reference LOCSOP126

### Initial Set-Up of the P6 Planetary Ball Mill

To compensate for the unbalance in the Fritsch P6 ball mill, the counter-weight must be adjusted accordingly, this is the weight of the filled grinding bowl(s) with the lid(s). There is a 250 ml grinding bowl and lid (3.1 kg) which will use 0.4 kg grinding balls. The counterweight has been set to 3.6 kg to account for this and sample volume and should not be adjusted unless necessary e.g. if the mill becomes imbalanced. See table in ‘Guidelines for milling’

### Operation of the P6 Planetary Ball Mill

1. The following check should be performed prior to using the P6 mill;
a. The rubber disc is in the bowl holder, rough side up. Rubber discs, which are damaged or flattened, need to be replaced.
b. The Teflon seal, which seals the lid and the grinding bowl is not worn or dirty—replace if necessary.
2. Place the grinding bowl within a walled container and add 400 g of grinding balls and deionised water (If required) should then be added to the grinding bowl and the lid placed on. NOTE: Whilst dry milling, it is possible to achieve a final fineness down to 20 μm. For milling down to submicron levels, a suitable barrier liquid is required e.g. deionised water. A ratio of approximately 1 part solid to 1 part liquid should be used.
3. The bowl and lid should be sealed twice round the outside with suitable tape.
4. The bowl must be clamped in place using the Safe-Lock holder.
a. The ends of the clamping bar must be underneath each side of the clamp supports.
b. The screw is tightened so that the grinding bowl is firmly held within the bowl holder.
c. Using the torque wrench, the top tightening screw and turn clockwise until a distinctive “click” has been heard—this should be done twice. The screw has now been tightened to the correct level and the torque wrench, should be removed.
d. Close the lever on the clamping bowl.
5. Close the lid of the mill slowly until a click is heard to lock the mill.
6. Set parameters to be used e.g. speed (rpm), time (in hours and minutes), pause breaks etc on the user interface and press go. Caution: Whilst ball milling, it is possible to generate a lot of heat inside the grinding bowl, some of this heat will be transferred through to the surface of the grinding bowl. Use a non-contact (infrared) temperature sensor, to check the outside temperature of the grinding bowl after milling, prior to handling to prevent burns.
7. Upon completion of the cycle, open the mill lid and lever on the safe lock mechanism, unscrew the clamp and carefully remove the griding bowl.
8. Release any pressure build up by piercing the tape sealing the bowl and lid with a pin.
9. Samples can be removed from the bowl and balls by using a brush (dry) or poured (liquid). If material sticks to the balls a sieve shaker can also be used to remove the bulk of sample from the balls.

### Guidelines for Milling

To prevent excess wear of the grinding set and reduce the amount of heat generated during milling, it recommended that the grinding bowl(s) is always filled to the minimum volume:

| | Useful working | Minimum |
|---|---|---|
| Grinding | capacity | sample volume |
| bowl size | (ml) | (ml) |
| 500 ml | 80-225 | 80 |
| 250 ml* | 30-125 | 30 |
| 80 ml | 10-30 | 10 |
| 45 ml | 3-20 | 3 |
| 12 ml | 0.5-5 | 0.5 |

*size of bowl used

Note: For low density samples, the minimum sample volume may need to be increased.

Never run the ball mill with the milling balls without sample material; this will damage both the grinding bowl and the milling balls.
Whilst dry milling, it is possible to achieve a final fineness down to 20 μm. For milling down to submicron levels, a suitable liquid needs to be used e.g. deionised water.
Whilst ball milling, it is possible to generate a lot of heat inside the grinding bowl, some of this heat will be transferred through to the surface of the grinding bowl. Use a noncontact (infrared) temperature sensor, to check the outside temperature of the grinding bowl after milling, prior to handling to prevent burns.

### Factors Influencing the Final Fineness of the Sample

1: The milling time—a longer milling time, will increase the fine fraction of the powder produced by the ball mill.
2: The milling speed—a higher speed will reduce the milling time and increase the fine fraction. The mill should not be used >500 rpm to prevent excessive vibration and movement of the mill.
3: The number and size of the grinding balls-pre-grinding with large grinding balls will reduce the coarse fraction. Smaller grinding balls can then be used to increase the fine fraction. 400 g grinding balls should be used at all time when milling.

### Cleaning of the Grinding Bowl and Milling Balls

Grinding bowl and balls can be cleaned using isopropyl alcohol. Balls can also be sonicated to ensure complete removal of sample.

### Embodiments

1. A method of producing osteoconductive pellets, the method comprising:
   i) providing powdered calcium phosphate material mixed with polymer particles to form a feedstock;
   ii) shaping the feedstock to form pellets,
   iii) sintering the pellets, wherein the sintering comprises:
a) a first stage heating at a temperature and time that is sufficient to burn off the polymer and leave calcium phosphate material pellets; and
b) a second stage heating at a higher temperature of between 950° C. and 1150° C. to sinter the remaining calcium phosphate material in the pellets, thereby forming the osteoconductive pellets with surface topography in the form of 0.2 to 20 micron grains.
2. The method according to embodiment 1, wherein the method further comprises step (iv) a hydrothermal treatment (HTT) of the osteoconductive pellets to enhance their surface topography.
3. The method according to embodiment 2, wherein the hydrothermal treatment comprises heating to a temperature of about 90° C. and 120° C. for between 20 and 30 hours.
4. A method of producing osteoconductive β-TCP granules; the method comprising:
   i) providing β-TCP granules;
   ii) adding α-TCP particles and water to the β-TCP granules to form seeded β-TCP granules; and
   iii) applying a hydrothermal treatment to the seeded β-TCP granules, wherein the hydrothermal treatment comprises heating to a temperature of between about 90° C. and 120° C. in the presence of water to enhance the surface topography of the β-TCP granules, thereby forming the osteoconductive β-TCP granules.
5. The method according to any of embodiments 2-3 wherein the hydrothermal treatment forms needle projections on the surface of the osteoconductive pellets; or the method according to embodiment 4 wherein the hydrothermal treatment forms needle projections on the surface of the osteoconductive granules.
6. The method according to any of embodiments 2-5 wherein the hydrothermal treatment comprises the addition of α-TCP particles and water; optionally with agitation.
7 The method according to any of embodiments 2-6 wherein the hydrothermal treatment is at a temperature of about 115° C., and optionally for 20 to 30 hours.
8. The method according to any of embodiments 2-3, or 5-7, further comprise a hardening step, wherein the hardening step comprises dry-heating the osteoconductive pellets; or the method according to any of embodiments 4-7 further comprise a hardening step, wherein the hardening step comprises dry-heating the osteoconductive granules.
9. The method according to any of embodiments 1-3, or 5-8, wherein the polymer particles comprise or consist of Ethylene Vinyl Acetate (EVA)).
10. The method according to any of embodiments 1-3, or 5-9, wherein the polymer particles are less than 1 mm in size.
11. The method according to any of embodiments 1-3, or 5-10, wherein the calcium phosphate material has an average particle size of 0.02-25 μm.
12. The method according to any of embodiments 1-3, or 5-11, wherein the feedstock comprises at least 72% w/w calcium phosphate material.
13. The method according to any of embodiments 1-3, or 5-12, wherein a pre-feedstock is provided for initial loading and hot melt exclusion prior to the feedstock, wherein the pre-feedstock comprises less calcium phosphate material, and more polymer particles, relative to the feedstock.
14. The method according to any of embodiments 1-3, or 5-13, wherein the second stage (b) heating is at a temperature of between 950° C. and 1100° C., optionally for 6-12 hours.
15. The method according to any of embodiments 1-3, or 5-14, wherein the second stage (b) temperature is about 1050° C. for 1-12 hrs.
16. The method according to any of embodiments 1-3, or 5-15, wherein the osteoconductive pellets are hollow pellets; optionally wherein the hollow pellets are tubular in structure having a lumen extending therethrough and open at each end.
17. The method according to any of embodiments 1-3, or 5-16, wherein the osteoconductive pellets have an aspect ratio of 0.75 to 3 (length to width).
18. The method according to any of embodiments 16-17, wherein the hollow pellets are tubular in structure having a lumen extending therethrough and having a wall thickness of at least 0.2 mm.
19. The method according to any of embodiments 1-3, or 5-18, wherein the osteoconductive pellets are macroporous.
20. The method according to any of embodiments 1-3, or 5-18, wherein the osteoconductive pellets are microporous; or the method according to any of embodiments 4-8, wherein the osteoconductive granules are microporous.
21. The method according to any of embodiments 4-8 or 20, wherein the osteoconductive granules are about 0.5 to 5 mm in size.
22 The method according to any of embodiments 1-3, or 5-20, wherein the method further comprises the addition of an active agent and/or cells to the osteoconductive pellets; or the method according to any of embodiments 4-8, or 20-21 wherein the method further comprises the addition of an active agent and/or cells to the osteoconductive granules.
23. The method according to any of embodiments 1-3, or 5-20, or 22 wherein the method further comprises the addition of the osteoconductive pellets to a carrier; or the method according to any of embodiments 4-8, or 20-22 wherein the method further comprises the addition of the osteoconductive granules to a carrier.
24. Osteoconductive pellets formed by the method according to any of embodiments 1-3, 5-20 or 22-23.
25. Osteoconductive granules formed by the methods according to any of embodiments 4-8, or 20-23.
26 An osteoconductive pellet, wherein the osteoconductive pellet is microporous and comprises or consists of at least 72% w/w calcium phosphate material, and optionally wherein the osteoconductive pellet comprises a hollow extending therethrough.
27 An osteoconductive granule, wherein the osteoconductive granule comprises or consists of a β-TCP granule, and further comprises a surface topography comprising needle structures projecting from the surface of the β-TCP granule.
28. A composition comprising osteoconductive pellets according to embodiments 24 or 26 and/or osteoconductive granules according to embodiments 25 or 27.
29. A method of treatment for bone repair or replacement, the method comprising the implantation of the osteoconductive pellets according to embodiments 24 or 26, and/or osteoconductive granules according to embodiments 25 or 27, or the composition according to embodiment 28, into a subject in need thereof.
30. The osteoconductive pellets according to embodiments 24 or 26, and/or osteoconductive granules according to embodiments 25 or 27, or the composition according to embodiment 28, for use as a medicament.
31. The osteoconductive pellets according to embodiments 24 or 26, and/or osteoconductive granules according to embodiments 25 or 27, or the composition according to embodiment 28, for use in bone repair or replacement in a subject.

1. A method of producing osteoconductive pellets, the method comprising:
(i) providing powdered calcium phosphate material mixed with polymer particles to form a feedstock;
(ii) shaping the feedstock to form pellets,
(iii) sintering the pellets, wherein the sintering comprises:
(a) a first stage heating at a temperature and time that is sufficient to burn off substantially all of the polymer and leave calcium phosphate material pellets; and
(b) a second stage heating at a higher temperature of between 950° C. and 1150° C. to sinter the calcium phosphate material pellets, thereby forming osteoconductive pellets with surface topography in the form of 0.2 to 20 micron grains.

2. The method according to claim 1, wherein the method further comprises step (c) applying a hydrothermal treatment to the osteoconductive pellets to enhance their surface topography.

3. The method according to claim 2, wherein the hydrothermal treatment comprises heating to a temperature of about 90° C. and 120° C. for between 20 and 30 hours.

4. A method of producing osteoconductive β-TCP granules; the method comprising:
(i) providing β-TCP granules;
(ii) adding α-TCP particles and water to the β-TCP granules to form seeded β-TCP granules; and
(iii) applying a hydrothermal treatment to the seeded β-TCP granules, wherein the hydrothermal treatment comprises heating to a temperature of between about 90° C. and 120° C. in the presence of water to enhance the surface topography of the β-TCP granules, thereby forming the osteoconductive β-TCP granules.

5. The method according to claim 2 or 3, wherein the hydrothermal treatment forms needle projections on the surface of the osteoconductive pellets.

6. The method according to claim 4, wherein the hydrothermal treatment forms needle projections on the surface of the osteoconductive granules.

7. The method according to any of claims 2 to 6, wherein the hydrothermal treatment comprises the addition of α-TCP particles and water; optionally with agitation.

8. The method according to any of claims 2 to 7, wherein the hydrothermal treatment is at a temperature of about 115° C., and optionally for 20 to 30 hours.

9. The method according to claim 2 or 3, or any of claims 5, 7 and 8 where dependent thereon, wherein the method further comprises a hardening step, wherein the hardening step comprises dry-heating the osteoconductive pellets.

10. The method according to claim 4 or 6, or claim 7 or 8 where dependent thereon, wherein the method further comprises a hardening step, wherein the hardening step comprises dry-heating the osteoconductive granules.

11. The method according to any of claims 1 to 3, or any of claims 5, 7, 8 and 9 where dependent thereon, wherein the polymer particles comprise or consist of ethylene vinyl acetate.

12. The method according to any of claims 1 to 3, or any of claims 5, 7 to 9, and 11 where dependent thereon, wherein the polymer particles are less than 1 mm in size.

13. The method according to any of claims 1 to 3, or any of claims 5, 7 to 9, 11 and 12 where dependent thereon, wherein the calcium phosphate material has an average particle size of 0.02-25 μm.

14. The method according to any of claims 1 to 3, or any of claims 5, 7 to 9, and 11 to 13 where dependent thereon, wherein the feedstock comprises at least 72% w/w calcium phosphate material.

15. The method according to any of claims 1 to 3, or any of claims 5, 7 to 9, and 11 to 14 where dependent thereon, wherein a pre-feedstock is provided for initial loading and hot melt exclusion prior to the feedstock, wherein the pre-feedstock comprises less calcium phosphate material, and more polymer particles, relative to the feedstock.

16. The method according to any of claims 1 to 3, or any of claims 5, 7 to 9, and 11 to 15 where dependent thereon, wherein the first stage (a) heating is at a temperature of at least about 500° C., but not more than 800° C., preferably wherein the first stage (a) heating is at a temperature of between about 500° C. and about 800° C. for at least 3 hours.

17. The method according to any of claims 1 to 3, or any of claims 5, 7 to 9, and 11 to 16 where dependent thereon, wherein the second stage (b) heating is at a temperature of between 950° C. and 1100° C., optionally for 6-12 hours.

18. The method according to any of claims 1 to 3, or any of claims 5, 7 to 9, and 11 to 17 where dependent thereon, wherein the second stage (b) temperature is about 1050° C. for 1-12 hrs.

19. The method according to any of claims 1 to 3, or any of claims 5, 7 to 9, and 11 to 18 where dependent thereon, wherein the osteoconductive pellets are hollow pellets; optionally wherein the hollow pellets are tubular in structure having a lumen extending therethrough and open at each end.

20. The method according to any of claims 1 to 3, or any of claims 5, 7 to 9, and 11 to 19 where dependent thereon, wherein the osteoconductive pellets have an aspect ratio of 0.75 to 3 (length to width).

21. The method according to claim 19 or 20, wherein the hollow pellets are tubular in structure having a lumen extending therethrough and having a wall thickness of at least 0.2 mm.

22. The method according to any of claims 1 to 3, or any of claims 5, 7 to 9, and 11 to 21 where dependent thereon, wherein the osteoconductive pellets are macroporous.

23. The method according to any of claims 1 to 3, or any of claims 5, 7 to 9, and 11 to 22 where dependent thereon, wherein the osteoconductive pellets are microporous.

24. The method according to claim 4 or 6, or claim 7 or 8 where dependent thereon, wherein the osteoconductive granules are microporous.

25. The method according to claim 4 or 6, or any of claim 7, 8 or 24 where dependent thereon, wherein the osteoconductive granules are about 0.5 to 5 mm in size.

26. The method according to any of claims 1 to 3, or any of claims 5, 7 to 9, and 11 to 23 where dependent thereon, wherein the method further comprises the addition of an active agent and/or cells to the osteoconductive pellets.

27. The method according to claim 4 or 6, or any of claim 7, 8, 24, or 25 where dependent thereon, wherein the method further comprises the addition of an active agent and/or cells to the osteoconductive granules.

28. The method according to any of claims 1 to 3, or any of claim 5, 7 to 9, and 11 to 23 or 26 where dependent thereon, wherein the method further comprises the addition of the osteoconductive pellets to a carrier.

29. The method according to claim 4 or 6, or any of claim 7, 8, 24, 25 or 27 where dependent thereon, wherein the method further comprises the addition of the osteoconductive granules to a carrier.

30. Osteoconductive pellets formed by the method according to any of claims 1 to 3, or any of claim 5, 7 to 9, 11 to 23, 26, or 28 where dependent thereon.

31. Osteoconductive granules formed by the methods according to claim 4 or 6, or any of claim 7, 8, 24, 25, 27 or 29 where dependent thereon.

32. An osteoconductive pellet, wherein the osteoconductive pellet is microporous and comprises or consists of at least 72% w/w calcium phosphate material, and optionally wherein the osteoconductive pellet comprises a hollow extending therethrough.

33. An osteoconductive granule, wherein the osteoconductive granule comprises or consists of a 6-TCP granule, and further comprises a surface topography comprising needle structures projecting from the surface of the β-TCP granule.

34. A composition comprising osteoconductive pellets according to claim 30 or 32 and/or osteoconductive granules according to claim 31 or 33.

35. A method of treatment for bone repair or replacement, the method comprising the implantation of the osteoconductive pellets according to claim 30 or 32, and/or osteoconductive granules according to claim 31 or 33, or the composition according to claim 34, into a subject in need thereof.

36. The osteoconductive pellet(s) according to claim 30 or 32, and/or osteoconductive granule(s) according to claim 31 or 33, or the composition according to claim 34, for use as a medicament.

37. The osteoconductive pellet(s) according to claim 30 or 32, and/or osteoconductive granule(s) according to claim 31 or 33, or the composition according to claim 34, for use in bone repair or replacement in a subject.
